(19) United States
(12) Patent Application Publication
(10) Pub. No.: US 2017/0218436 A1
(43) Pub. Date: Aug. 3, 2017

(54) DIAGNOSTIC, PROGNOSTIC, AND ANALYTICAL SYSTEM

(71) Applicant: KIMIYA PTY. LTD., Blaxland New South Wales (AU)

(72) Inventors: Mehdi Azimi, Linfield New South Wales (AU); Matthew Taylor Worsman, Blaxland New South Wales (AU); Sara Alavioon, Linfield New South Wales (AU); Simone Charlotte Vonwiller, Lane Cove New South Wales (AU)

(21) Appl. No.: 15/328,839

(22) PCT Filed: Jul. 24, 2015
(86) PCT No.: PCT/AU2015/050420

§ 371 Date: Jan. 24, 2017

Related U.S. Application Data
(60) Provisional application No. 62/034,815, filed on Aug. 8, 2014.

(30) Foreign Application Priority Data
Jul. 7, 2015 (IB) PCT/IB2015/055117

(51) Int. Cl. C12Q 1/68; B01L 7/00; A61B 10/00; B01L 3/00

(52) U.S. Cl. CPC C12Q 1/6818; B01L 3/502715; B01L 3/50273; B01L 3/502738; B01L 7/52; B01L 3/502707; C12Q 1/6825; A61B 10/0041; A61B 10/0045; B01L 2400/0478; B01L 2300/0877; B01L 2300/1805; B01L 2200/14; B01L 2300/0654; B01L 2400/0633; B01L 2300/023; B01L 2200/12; B01L 2300/0887

(57) ABSTRACT

A device for analyzing a liquid sample. The device includes an inlet for receiving the sample, a reaction chamber, an analysis module, and at least one pump for moving fluid within the one or more flow paths. The device includes one or more flow paths arranged so as to provide a fluid flow path between the inlet and the reaction chamber, and a fluid flow path between the reaction chamber and the analysis module. The device may be used for analyzing a liquid sample, such as, but not limited to nipple aspirate fluid (NAF).

### FIELD OF THE INVENTION

The present invention relates to diagnostic, prognostic, and analytical systems/devices that incorporate “micro total analysis system” (μTAS) technologies or the like. In particular, the invention relates to processing of a sample and interacting with the sample via fluidic, biochemical, chemical, electrical, optical, optoelectronic, and electronic means for the purpose of achieving diagnostic, prognostic, and analytical objectives.

### RELATED US APPLICATION DATA

Provisional application No. 62034815, filed on 8 Aug. 2014. The country code and number of the priority application, to be used for filing abroad under the Paris Convention, is U.S. 62/034,815.

### BACKGROUND OF THE INVENTION

Breast cancer is the most common form of cancer contracted by women worldwide. In many regions of the world, it is the leading cause of cancer-related deaths in women.

A significant but not surprising statistic is that the survival rate relates directly to the size of the tumor with a five-year relative survival of 98.2% for 0 mm to 10 mm tumors, 94.7% for 11 mm to 15 mm tumors, 93% for 16 mm to 19 mm tumors, 87.9% for 20 mm to 29 mm tumors and 73.1% for tumors larger than 30 mm (The Australian Cancer Council, 2014). Therefore, it is clear that early diagnosis can increase survival rates. Given the high correlation between survival rate and the early detection of breast cancer, it is crucial to detect abnormal cellular function before the onset of significant and deleterious physiological changes. Early detection of breast cancer is considered essential for breast cancer control.

Conventional radiological diagnostic techniques focus on detecting the physical manifestations of breast cancer. Conventional breast cancer screening techniques of mammography and breast examination can miss early breast cancer, particularly if the tumor is anatomically insignificant. These conventional techniques are less effective in detecting cancer in young women owing to their increased breast tissue density, with the value of mammographic screening for women under 50 acknowledged to be very uncertain. Conventional screening techniques are also susceptible to over-diagnosis and consequently over-treatment. Conventional screening techniques must also rely on a biopsy for diagnostic data.

Invasive breast cancer mostly develops from tumors that manifest in the epithelial tissue lining the ductal-lobular unit of the breast. In non-lactating women, the ductal-lobular unit of the breast contains fluid. This fluid, commonly referred to as nipple aspirate fluid (NAF), contains exfoliated epithelial and hematogenous cells as well as proteins that have been secreted and concentrated into the duct lumen. Secreted substances are both endogenous and exogenous substances.

Nipple aspiration is a noninvasive, painless technique for yielding NAF from non-lactating women that is both low cost and repeatable. The aspiration technique most commonly used involves the massage of the breast from the chest out toward the nipple with the simultaneous application of suction upon the nipple-areolar complex.

Aspiration of NAF to the surface of the nipple provides a small liquid sample for analyzing the cells, proteins and other analytes of the ductal-lobular micro-environment to identify diagnostic and prognostic targets indicative of normal or abnormal processes, conditions or diseases, as well as how well the body is responding to treatment. Diagnostic and prognostic targets, such as altered genes, RNA products, proteins or other metabolites, allow for the screening of breast cancer prior to tumors becoming anatomically significant. Further, as breast cancer is a heterogeneous disease, these diagnostic and prognostic targets enable tumor profiling which in the case of conventional screening techniques requires invasive biopsy. Such profiling is a necessity for personalized medicine.

Especially suited for providing the means of analyzing small samples of fluid, such as NAF, are the “micro total analysis system” (μTAS) technologies. These technologies offer a range of nanometer-scale to millimeter-scale features that enable inexpensive, reliable, quick, convenient, and widely accessible point-of-care systems for identifying diagnostic and prognostic targets.

In view of the above, there is a need for diagnostic, prognostic, and analytical systems that incorporate micro total analysis system (μTAS) technologies or the like, in particular, systems processing a sample and interacting with the sample via fluidic, biochemical, chemical, electrical, optical, optoelectronic, and electronic means for the purpose of achieving diagnostic, prognostic, and analytical objectives.

### SUMMARY OF THE INVENTION

In one broad form, the present invention provides a device for analyzing a liquid sample, the device including: an inlet for receiving the sample; a reaction chamber; an analysis module; one or more flow paths arranged so as to provide a fluid flow path between the inlet and the reaction chamber, and a fluid flow path between the reaction chamber and the analysis module; and at least one pump for moving fluid within the one or more flow paths.

In a further broad form, the present invention provides a device for analyzing a liquid sample, the device including: a disposable part including: an inlet for receiving the sample; a reaction chamber; an analysis module; one or more flow paths arranged so as to provide a fluid flow path between the inlet and the reaction chamber, and a fluid flow path between the reaction chamber and the analysis module; and at least one pump for moving fluid within the one or more flow paths; and a reusable part that is releasably engageable with the disposable part, the reusable part including at least one actuator for operating the at least one pump of the disposable part.

In one form, the at least one pump is at least one syringe pump, including a syringe barrel.

In another form, one of the at least one syringe barrels forms the reaction chamber.

In a further form, the device includes a heater to heat the contents of the reaction chamber.

In another form, the device includes a syringe pump heating sleeve to heat the contents of the reaction chamber.

In one form, the device includes at least one sensor configured to detect the presence or absence of liquid within the one or more flow paths.

In another form, one of the at least one sensors is configured to detect the presence or absence of liquid at a point on the flow path between the inlet and the reaction chamber.

In one form, one of the at least one sensors is configured to detect the presence or absence of liquid at a point on the flow path between the reaction chamber and the analysis module.

In a further from, the analysis module is configured to detect the presence or absence of at least one analyte in the sample.

In another form, the analysis module includes at least one photosensor.

In another form, the analysis module is configured to detect the presence or absence of the at least one analyte using electrochemiluminescence.

In a further form, the analysis module includes one or more detection chambers.

In another form, one or more of the detection chambers includes an electrochemiluminescence resonance energy transfer probe.

In one form, the analysis module includes at least one heater to heat the contents of one or more of the detection chambers.

In another form, the analysis module includes a plurality of detection chambers.

In one form, the analysis module includes at least 47 detection chambers.

In another form, each detection chamber includes a probe specific to a particular analyte.

In a further form, each detection chamber includes a valve closable to seal the detection chamber.

In a further form, the valve is a hydrogel valve.

In one form, the valve is temperature-responsive.

In another form, the sample includes Nipple Aspirate Fluid (NAF).

In one form, the at least one analyte is a diagnostic marker, prognostic marker, disease marker, nucleic acid, protein, or peptide.

In one form, the device includes at least 2 pumps.

In another form, one of the at least two pumps is operable to draw fluid via suction through the analysis module.

In one form, the flow path between the inlet and the reaction chamber includes a dry reagent chamber.

In another form, the device further includes a communication interface that permits data transfer between the device and a processing system.

In one form, the device generates one or more signals indicative of the presence or absence of the at least one analyte, the one or more signals to be received by the processing system.

In another form, the device is configured to amplify a nucleic acid or part thereof in the reaction chamber

In one form, the device is configured to amplify the nucleic acid via an isothermal amplification reaction.

In another form, the device is configured to perform an assay utilizing an amplification master mix, the amplification master mix including an enzyme initially preloaded in the device in dry form.

In a further form, the preloaded enzyme is preloaded in the reagent chamber.

In one form, the amplification reaction is carried out at a temperature range of between 30° C. to 65° C.

In another form, the amplification is carried out at a temperature range of between 55° C. to 59° C.

In one form, the amplification master mix, includes a nicking enzyme.

In another form, for a given sample analysis, the device is configured such that the amplification reaction is carried out for a period of 10 minutes or less.

In a further form, wherein the analysis module is produced by a microfabrication process, and the analysis module includes: a substrate; and a patterned hydrogel layer that provides one or more hydrogel valve actuators.

In a further form, wherein the analysis module further includes: a cap foundation layer including a deep ultraviolet (DUV) photopatternable thermoplastic layer; and a chip cap including a thermoplastic layer; wherein the substrate includes a silicon layer.

In a further broad form, the present invention provides a device for analyzing a liquid sample, wherein the device is configured to analyze the sample, the sample including Nipple Aspirate Fluid (NAF).

The diagnostic, prognostic, and analytical systems, according to the present invention, integrate and automate the procedure for achieving diagnostic, prognostic, and analytical objectives. The present invention may incorporates micro total analysis system (μTAS) technologies or the like that provide for high system performance regarding early diagnosis, high system portability, and very low system cost.

### BRIEF DESCRIPTION OF THE DRAWINGS

Preferred embodiments of the present invention will now be described by way of example only with reference to the accompanying drawings, in which:

FIG. 1 shows an embodiment of the assay device with the protective cap removed;

FIG. 2 shows the top, side, front, and back views of an embodiment of the assay device without the protective cap;

FIG. 3 shows the top and side cross-sections of an embodiment of the assay device;

FIG. 4 shows the layout of an embodiment of the analysis chip;

FIG. 5 shows the assay device in use;

FIG. 6 shows the schematic of an embodiment of the assay device;

FIG. 7 shows a flow diagram describing the operation of an embodiment of the assay device;

FIG. 8 shows a simplified layout of an embodiment of the analysis chip;

FIG. 9 shows the detection chamber;

FIG. 10 shows the detection chamber cross-section;

FIG. 11 shows a simplified layout of the second embodiment of the analysis chip;

FIG. 12 shows the first embodiment of the integration chip;

FIG. 13 shows the third embodiment of the analysis chip;

FIG. 14 shows the fourth embodiment of the analysis chip;

FIG. 15 shows the layout of the detection chamber of the type used in the second embodiment of the analysis module;

FIG. 16 shows the cross-section B, as defined in FIG. 15, of the detection chamber of the type used in the second embodiment of the analysis module;

FIG. 17 shows a schematic cross-sectional depiction of the base wafer at the end of the first phase of the microfabrication process;

FIG. 18 shows the passivation layer after being deposited on the base wafer, planarized and patterned to open the contact holes in the passivation layer;

FIG. 19 shows the first transducer layer after being deposited on the base wafer and patterned to create the heaters and the electrodes;

FIG. 20 shows the passivation layer after being patterned again to open the bond pad openings in the passivation layer;

FIG. 21 shows the cap foundation layer after being deposited on the base wafer and patterned to create a foundation for a chip cap;

FIG. 22 shows the hydrogel layer after being deposited on the base wafer and patterned;

FIG. 23 shows the mold substrate;

FIG. 24 shows the mold roof layer after being deposited on the mold substrate and exposed;

FIG. 25 shows the mold wall layer after being deposited on the mold substrate and exposed, and after the mold roof layer and the mold wall layer being developed;

FIG. 26 shows the elastomer layer after being dispensed on the mold after the stamp release layer having been dispensed on the mold, and subsequently cured;

FIG. 27 shows the stamp after being released, ready to be used in the fabrication of the chip cap;

FIG. 28 shows the thermoplastic after having been dispensed on the handle wafer and subsequently hot-embossed with the stamp to form the chip cap;

FIG. 29 shows the chip cap attached to the handle wafer;

FIG. 30 shows the chip cap being solvent-bonded to the base wafer, forming a stack of the base wafer, the chip cap, and the handle wafer;

FIG. 31 shows the base wafer, the chip cap, and the handle wafer after having been mounted on a film frame and after the handle wafer having been released;

FIG. 32 shows a schematic of the second embodiment of the assay device;

FIG. 33 shows a flow diagram describing the operation of the second embodiment of the assay device;

FIG. 34 shows the continuation of the flow diagram describing the operation of the second embodiment of the assay device;

FIG. 35 shows the continuation of the flow diagram describing the operation of the second embodiment of the assay device;

FIG. 36 shows a schematic of third embodiment of the assay device;

FIG. 37 schematically shows the plan view of the fifth embodiment of the analysis chip;

FIG. 38 shows a schematic cross-section of the analysis chip assembly comprising the fifth embodiment of the analysis chip and an embodiment of the analysis chip suction manifold;

FIG. 39 schematically shows the plan view of the sixth embodiment of the analysis chip;

FIG. 40 shows a schematic cross-section of the analysis chip assembly comprising the sixth embodiment of the analysis chip and an embodiment of the analysis chip suction manifold;

FIG. 41 shows a flow diagram describing the operation of the third embodiment of the assay device;

FIG. 42 shows a schematic of the fourth embodiment of the assay device;

FIG. 43 shows a schematic of the fifth embodiment of the assay device;

FIG. 44 shows a depiction of the optical liquid sensor;

FIG. 45 depicts a deployment model of the diagnostic, prognostic, and analytical system; and,

FIG. 46A, FIG. 46B, and FIG. 46C depict some of the probe types utilized in the analysis modules.

### DETAILED DESCRIPTION

Embodiments of the invention provide a device for analyzing a liquid sample such as Nipple Aspirate Fluid. The device may be configured to perform assays and, therefore, may be considered an assay device. Typically, the device includes an inlet for receiving the sample, a reaction chamber, and an analysis module. The analysis module may, for example, include or be part of an analysis chip. Typically, the analysis module will include one or more detection chambers for detecting particular analytes.

One or more flow paths are arranged to provide a fluid flow path between the inlet and the reaction chamber, and to provide a fluid flow path between the reaction chamber and the analysis module. Typically, at least one pump is utilized for moving fluid (e.g., sample) through the one or more flow paths.

Typically, the device is configured such that the sample is first directed toward the reaction chamber to prepare/isolate an analyte from the sample for analysis. This may include one or more reactions such as, for example, nucleic acid amplification reactions. Generally, once the analyte is prepared it is directed to the analysis module (e.g., analysis chip) where detection of the presence or absence of the analyte occurs. The analyte may, for example, be a diagnostic marker (e.g., a disease marker) and/or a prognostic marker.

The device therefore has advantages in that preparation of the sample/analyte and analyte detection are carried out in a single device. This allows point of care analysis of patient samples without the need for a lab or transport of the sample thereto.

Some particular preferred embodiments of the invention are described as follows.

### Overview and the Deployment Model

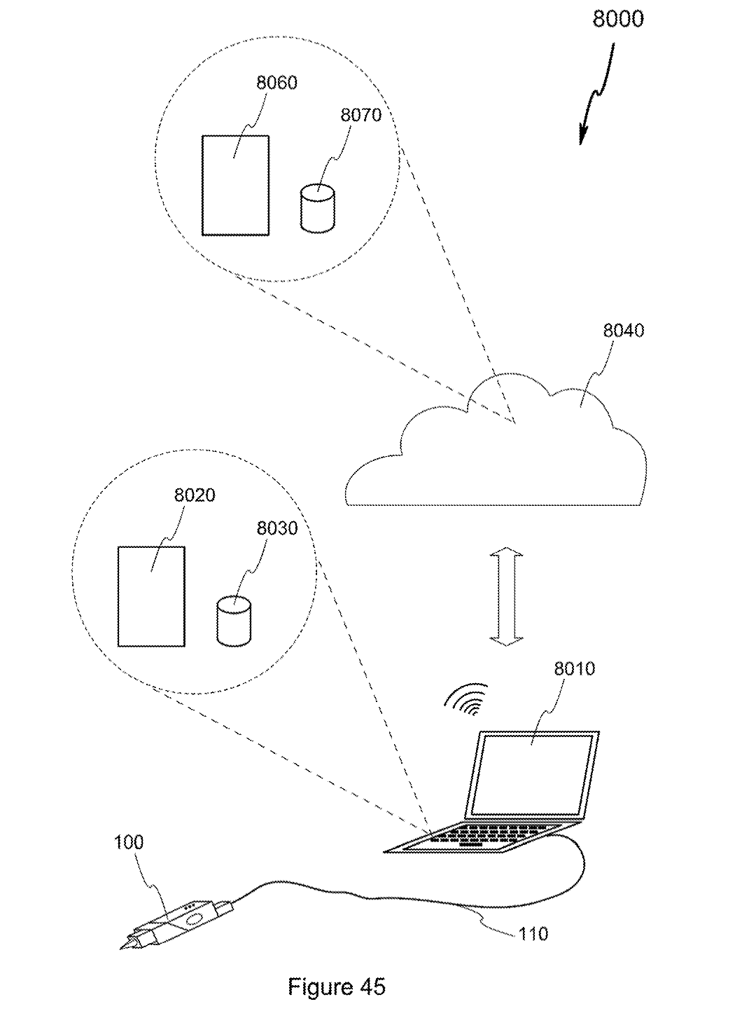

A typical deployment model 8000 of the diagnostic, prognostic, and analytical system is depicted schematically in FIG. 45. The diagnostic, prognostic, and analytical system comprises one or more assay devices 100, one or more hosts 8010, application software 8020, application software database 8030, support application 8060, and a master database 8070. Other embodiments of the assay device may be used in place of or in addition to the assay devices 100.

The assay device 100 and other embodiments of the assay device are devices that are used to collect a fluid sample and perform analysis of the fluid sample with respect to various diagnostic, prognostic, and analytical objectives.

The host 8010 is a laptop computer, a desktop computer, a tablet computer, a smart phone, or any other processing system that can provide a suitable level of information processing capability for the assay device, can suitably communicate with the assay device, and can supply power to the assay device. As an information processing system, the host receives user inputs, monitors the status of the assay device, controls the assay device, collects the assay results generated by the assay device, processes the raw assay results to generate diagnostic, prognostic, and analytical results, and presents the results to the user and receives the user inputs via a user interface. The host 8010 is preferably connected to the outside world, e.g., to internet or a cloud infrastructure 8040, via one or more data links, which the host may utilize to interact with the outside world, for example, to update the application software 8020 or the application software database 8030 on the host or on the assay device (if the said embodiment of the assay device incorporates application software or databases).

The application software 8020 preferably resides on the host 8010 and is responsible for driving the assay device 100 or other assay device embodiments and for providing user interface. The user can use the user interface of the application software on the host to view the results of the analysis of the sample, can view the status of the system, can provide inputs to the system, etc.

The application software database 8030 provides a data repository for the data utilized by the application software, e.g., list of probes, list of markers, tables defining the relationships between the probes and the markers, etc.

The support application 8060 is preferably hosted on the cloud infrastructure 8040 and provides for global updating of the application software 8020 and the application software database 8030 on the hosts 8010.

The master database 8070 is preferably hosted on the cloud infrastructure 8040 and is used for updating the application software database 8030.

### The Assay Device

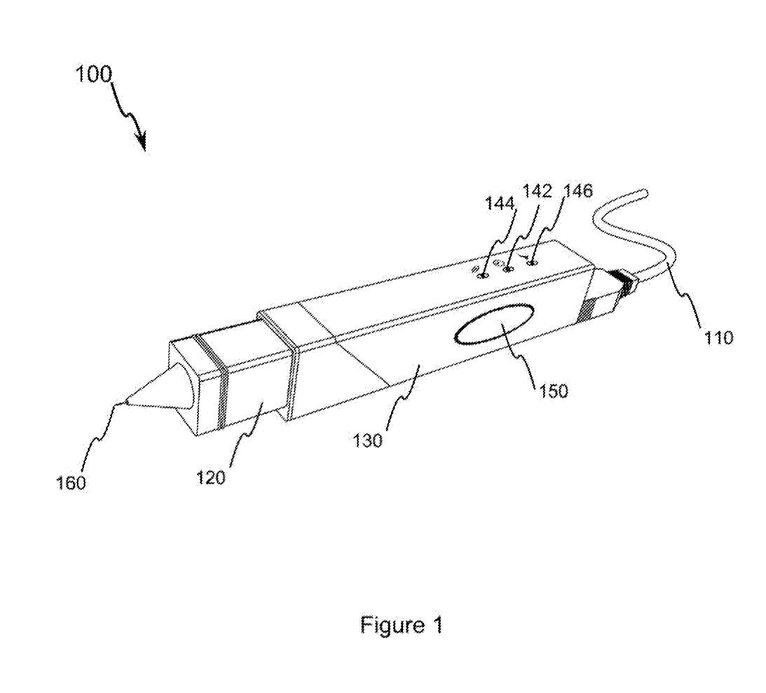

The assay device 100 is one of the assay device embodiments. A depiction of the assay device 100 is given in FIG. 1. As previously described, the assay device includes a communication interface that permits communication/data transfer between the assay device and a processing system; the processing system may be the host 8010. In many of the embodiments of the assay device 100, a USB link is used for connecting the assay device to the host 8010 (see FIG. 45). A standard USB cable 110 is preferably used for this purpose. It will be appreciated that in other examples, the assay device may communicate wirelessly (e.g., via Wi-Fi, Bluetooth, etc.) with the host/processing system.

The assay device 100 is a handheld integrated system. As part of the diagnostic, prognostic, and analytical system, the assay device 100 is used to collect a fluid sample and to perform analysis of the fluid sample with respect to various diagnostic, prognostic, and analytical objectives. In the preferred embodiments of the invention, the fluid sample collected by the assay device 100 is a liquid sample; in this context, the liquid sample is defined as any form of the fluid sample that is capable of forming a free surface, e.g., an emulsion, a suspension, water, nipple aspirate fluid (NAF), breast milk, saliva, or blood. The liquid sample collected by the assay device 100 can be a substance that is originally in the form of a fluid that is capable of forming a free surface, or it can be a sample that is liquefied from a nonfluid form according to one of the standard methods known to a skilled worker in the art. The analysis performed by the diagnostic, prognostic, and analytical systems can involve a variety of medical diagnostic, biological, biochemical, or chemical tests. This specification primarily describes the assay device for screening patients for various forms of breast cancer using a NAF sample as the liquid sample type. There are many different embodiments of the assay device, but the description that follows will initially concentrate on the assay device 100.

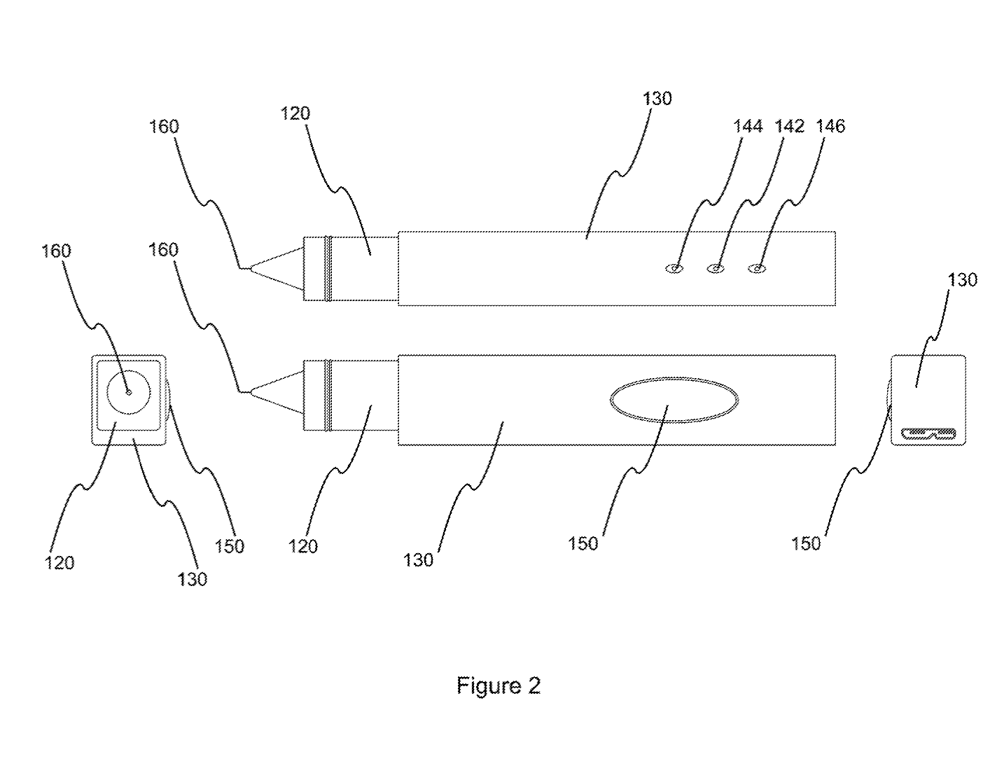

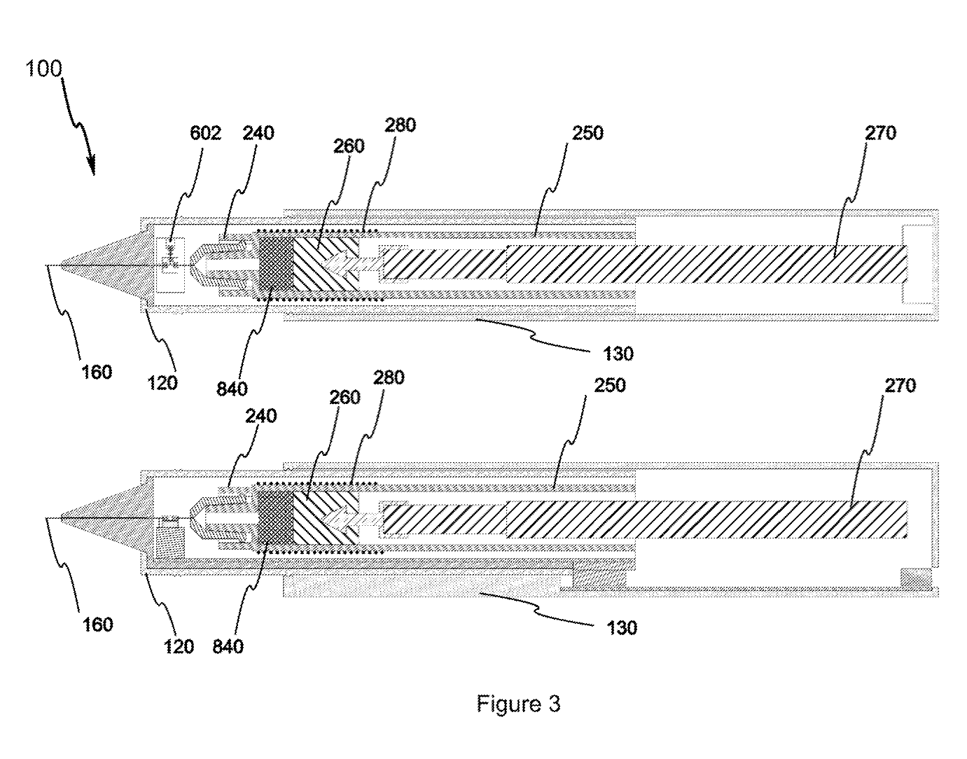

Top, side, front, and back views of the assay device 100 are shown in FIG. 2, and a top cross-section and a side cross-section of the assay device 100 are shown in FIG. 3.

During operation, the assay device 100 is connected to the host 8010 via a link that is capable of providing power to the assay device and providing bidirectional communication between the host and the assay device.

In the following sections, the design and the operation of the assay device will be discussed in detail.

### Assay Device Design

Referring to FIG. 1 through FIG. 4, the design of assay device 100 will be described next. The assay device incorporates fluidic, mechanical, electronic, and optoelectronic subsystems. Structurally these subsystems are divided between two main parts or blocks incorporated in the assay device 100: an expendable part/block 120 and a reusable part/block 130. The reusable block 130 is releasably engageable with the disposable block 120.

The expendable block 120 incorporates a capillary tube inlet 160 for collecting the liquid sample, fluidic, mechanical, electronic, and optoelectronic subsystems, and electronic and mechanical interfaces to the reusable block 130. The expendable block also has a protective cap (not shown) that protects the capillary tube inlet 160 of the module before use.

The reusable block 130 incorporates electronic and mechanical interfaces to the expendable block 120, a Micro-USB device port and its associated electronics for connection to the host, and mechanical and electronic subsystems to support the expendable block 120. The reusable block also incorporates a tactile switch 150 (push-button switch) and a number of light-emitting diodes (LEDs), e.g., a red LED 142, a green LED 144, and a blue LED 146; the tactile switch and the LEDs form part of the system's user interface.

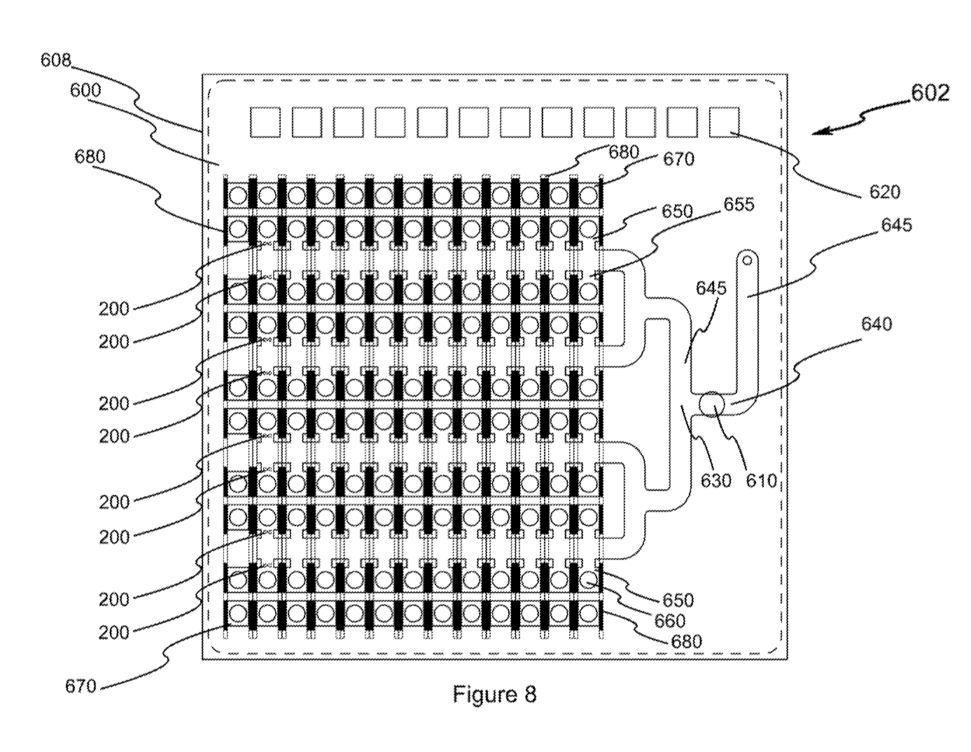

An important function in the assay device 100 is performed by an analysis module 600 (shown in FIG. 8). The analysis module 600 receives a fluid that can contain nucleic acids or proteins. The analysis module 600 then detects various target molecules (target nucleic acid sequences or various target proteins), corresponding to various diagnostic markers and/or prognostic markers, contained in the fluid. The said fluid that contains nucleic acids can contain amplified nucleic acids (amplicon). The analysis module 600 is part of the expendable block 120, and the analysis module 600 incorporates integrated fluidic, microelectromechanical, electronic, and optoelectronic subsystems. The details of the analysis module 600 will be described later in this specification.

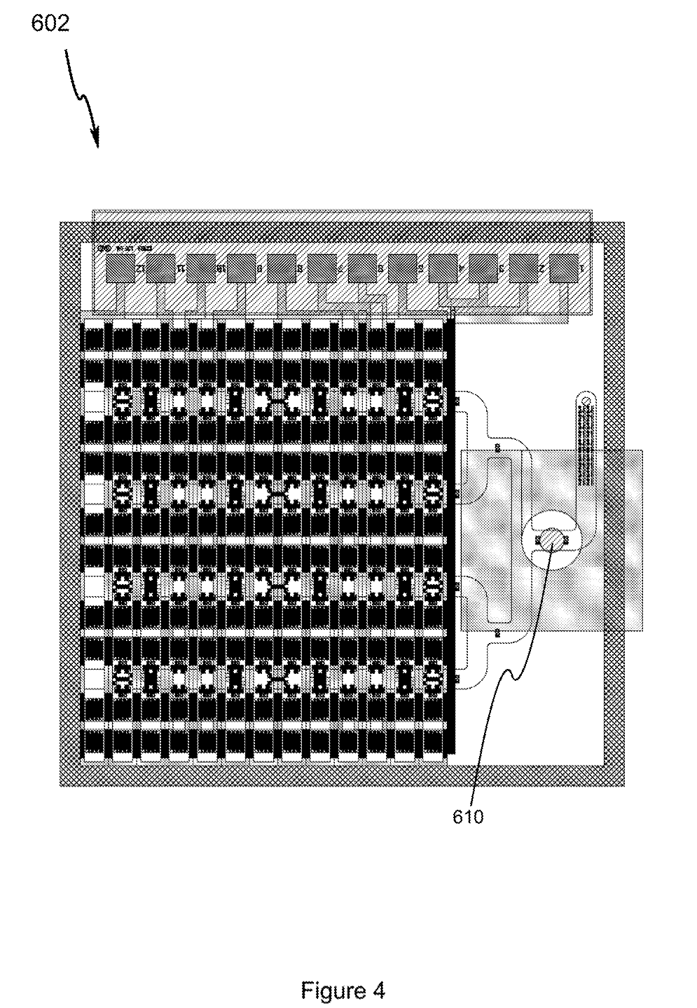

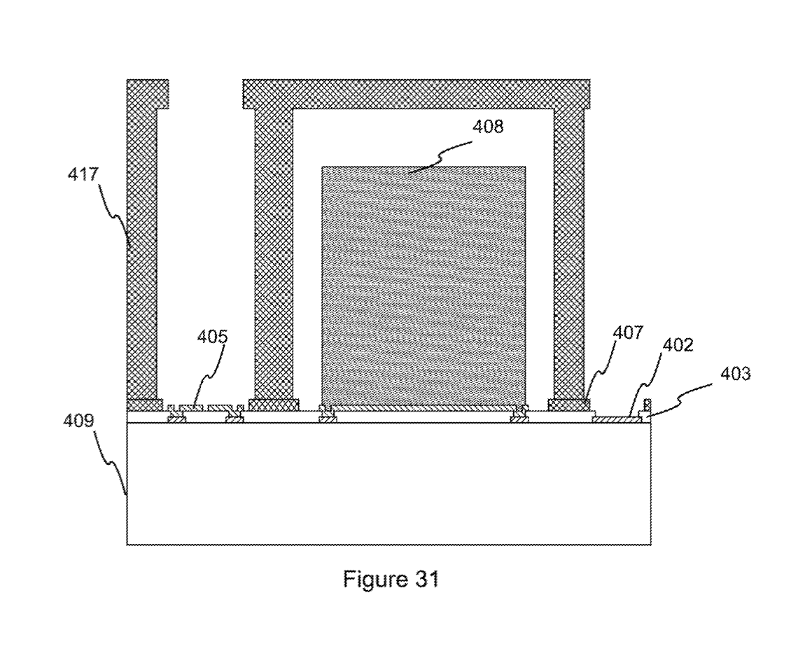

In the preferred embodiment of the invention, the analysis module 600 is incorporated in an analysis chip 602. The analysis chip 602 as part of the assay device 100 is shown in FIG. 3. A layout of the analysis chip 602 is shown in FIG. 4. In one embodiment, the analysis chip 602 is fabricated on a substrate 409 (best shown in FIG. 31) using microfabrication process steps that will be described later in this specification. The substrate 409 comprises a layer of silicon, quartz, or other suitable material. Other embodiments of the analysis chip 602, the analysis chip 3510 (to be described later in this specification), the analysis chip 7010 (to be described later in this specification), the analysis chip 7510 (to be described later in this specification), many other embodiments of the analysis chip, and various embodiments of the integration chip (to be described later in this specification) are fabricated on substrates comprising one or more of PMMA (poly(methyl methacrylate)), COC (cyclic olefin copolymer), or other suitable polymer. In other embodiments of the invention, an analysis module may include one or more fluidic, microelectromechanical, electronic, or optoelectronic chips and/or one or more discrete fluidic, microelectromechanical, electronic, or optoelectronic components.

### Assay Device Use

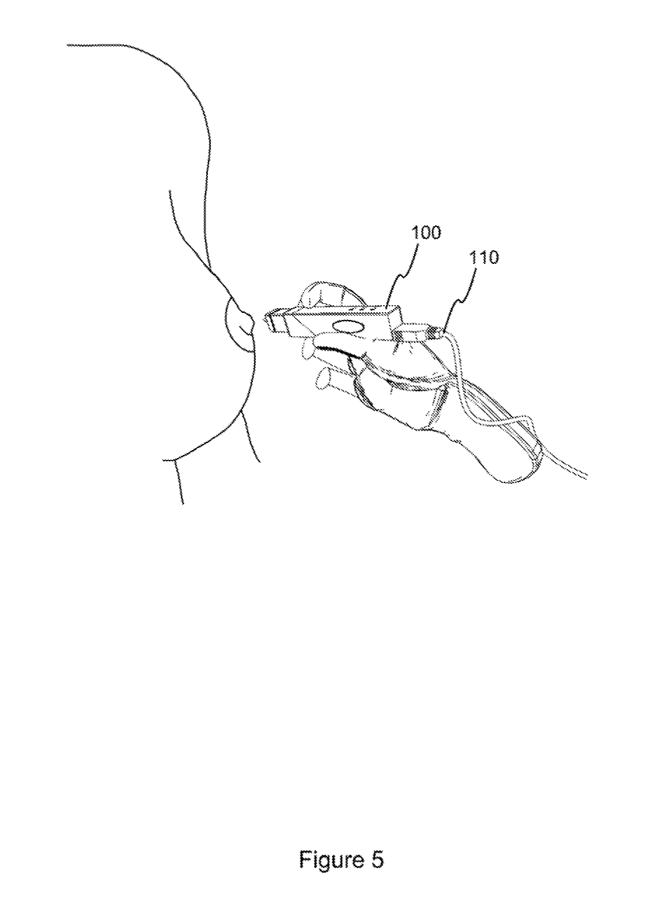

One variant of use of the assay device 100 will be described next. Other embodiments of the assay device, e.g., an assay device 500 and an assay device 800, may be used in a similar role in place of the assay device 100. These embodiments will be described later in this specification. To use the assay device 100, the expendable block 120 is taken out of its pouch and is inserted into the reusable block 130 (see FIG. 1, FIG. 2, and FIG. 5). The pouch is preferably a sterile pouch. The connector of the reusable block 130 is connected to the USB port on the host preferably via a standard USB cable 110. The user can now use the assay device 100 to collect a sample and proceed with the sample analysis. While FIG. 5 shows the assay device 100, other embodiments of the assay device, e.g., the assay device 500 and the assay device 800, may be used in place of the assay device 100.

For the NAF sample, the NAF is aspirated according to one of the standard NAF aspiration procedures (see for example Suikerbuijk, K. P. M., van Diest, P. J., van der Wall, E. Improving early breast cancer detection: focus on methylation. Annals of Oncology 2011, 22, 24-29). One of the methods in the use of the assay device is contralateral analysis, where two of the reusable blocks 120 of the assay device 100 are used, one for each breast, to perform analysis of the NAF aspirated from each of the breasts. A comparative analysis or a differential analysis of the results of the analysis for the left and right breasts can provide improved diagnostic, prognostic, and analytical outcomes.

As has been noted earlier in this specification, embodiments of the assay device can be used to perform analysis of sample types other than the NAF, e.g., water, breast milk, saliva, blood, and/or various chemicals. In these cases, the samples are introduced in the same manner as for the NAF. One or more of the liquid reagents, the assay reagents, and the probes are changed to those specific for the analytes of interest. Assay systems preferably detect the analytes of interest via emission from a reporter luminophore/fluorophore. The scope is broad and can include simple analytes such as glucose and ions to more complex examples such as proteins and nucleic acids. With these modified platforms, the reaction chamber is the site where the analyte is optionally isolated, enriched, and/or modified, and the detection chambers functions as assay chambers where the analytes interact with receptors to induce a signal. In that regard, the assay device is also a generic platform that is tailored to a wide range of analytical tasks by optionally modifying the chemistry.

The green LED 144 on the assay device indicates to the user that the assay device can be used to collect the sample. If this LED is turned off, it is an indication that the user should wait until that LED is turned on again. When the desired quantity of the sample is collected, the user can depress the tactile switch 150, after which, the assay device performs the assay. Instead of the user commanding the assay device to perform the assay by depressing the tactile switch 150, the user can command the assay device to perform the assay by using the user interface of the application software on the host. While the assay device is performing the assay, the red LED 142 lights up, indicating that the user has to wait for the assay to complete. After the assay is completed, the blue LED 146 lights up signaling the assay's completion. At this stage, the assay outcome can be obtained from the host's user interface.

The skilled worker in the art will readily recognize that the choice of colors, the arrangement, and the number of LEDs in the design of the assay device 100 can be varied without departing from the spirit and scope of the broad inventive concept.

### Assay Device Design and Operation

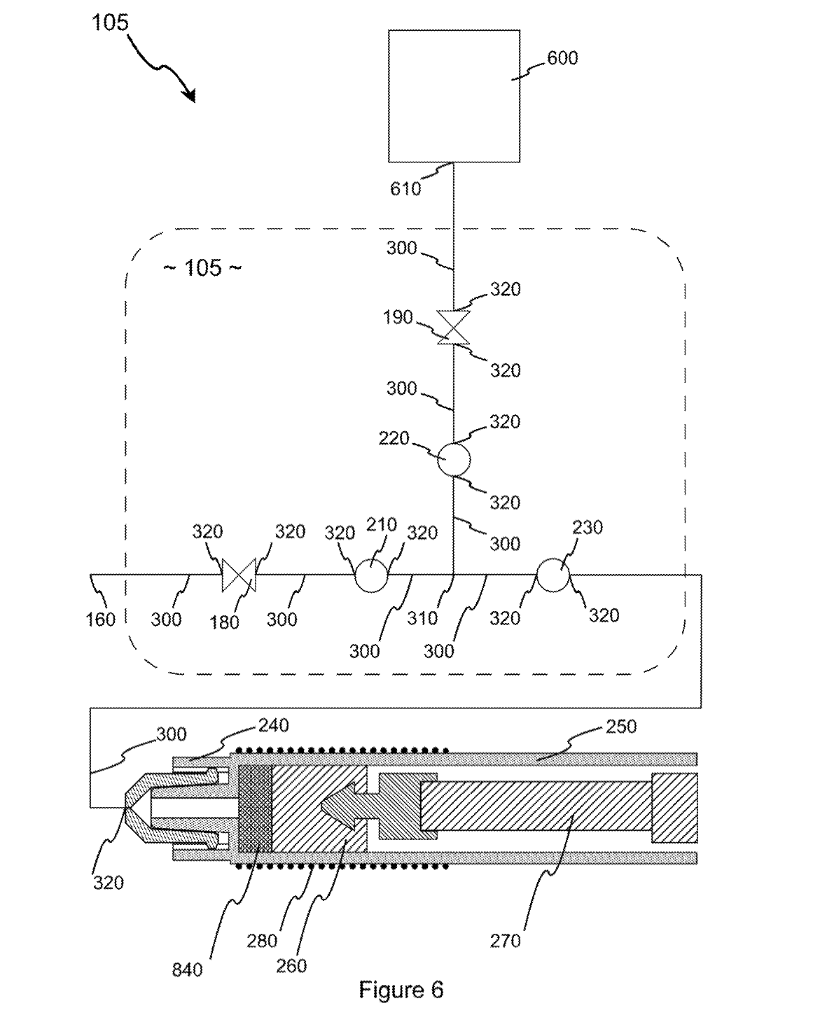

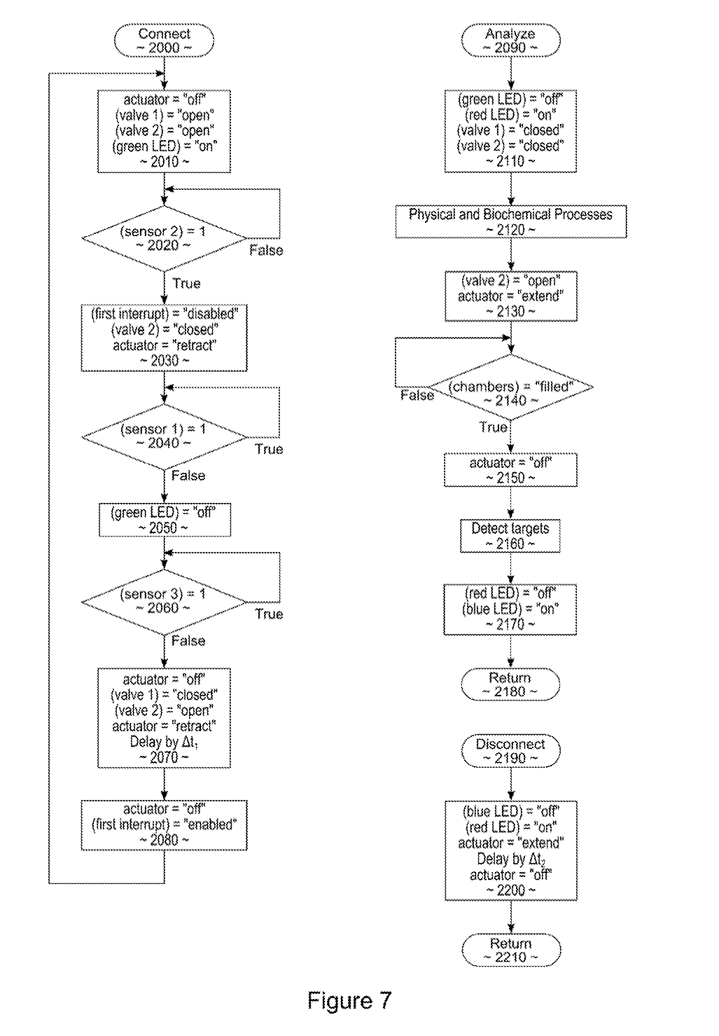

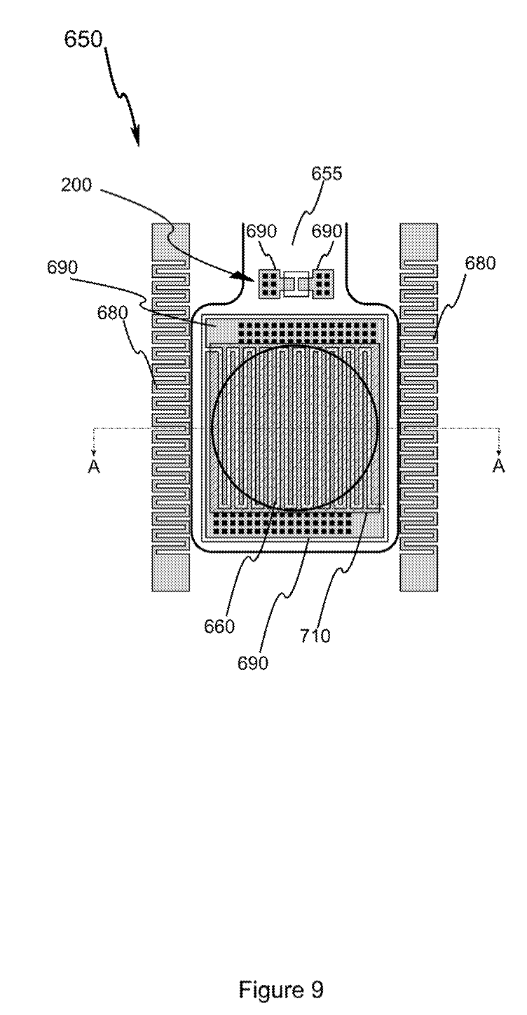

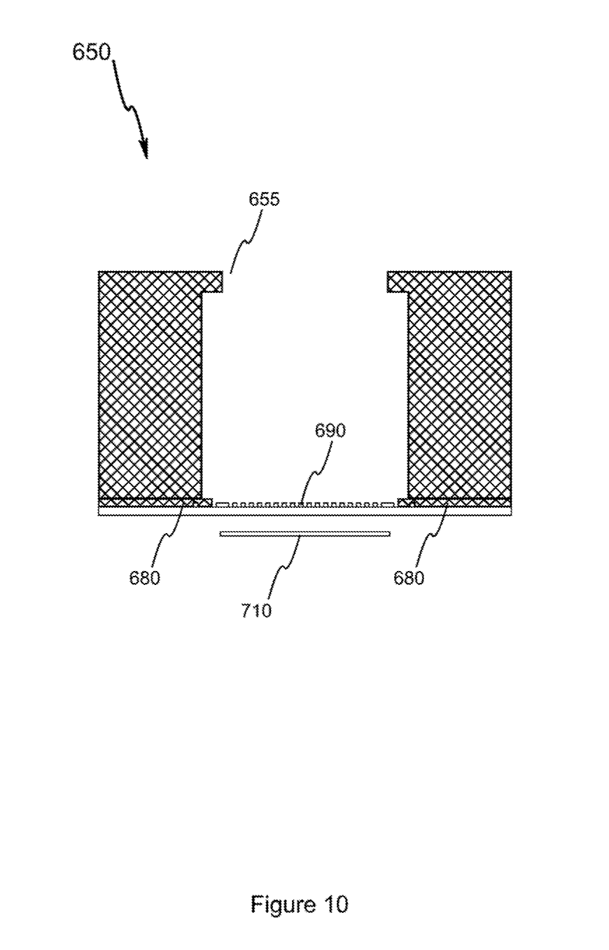

Referring to FIG. 3, FIG. 6, FIG. 7, FIG. 8, FIG. 9, and FIG. 10, the design and operation of the assay device 100 will be described.

A schematic of the assay device 100 is shown in FIG. 6. Referring to FIG. 3 and FIG. 6, the components of the assay device 100 that are incorporated in the expendable block 120 include a capillary tube inlet 160, a first valve 180, a second valve 190, a first liquid sensor 210, a second liquid sensor 220, a third liquid sensor 230, a syringe pump 240, a syringe pump heating sleeve 280, an analysis module 600, a vent (not shown), and a plurality of fluidic interconnections 300 between these components. The syringe pump 240 in turn incorporates a syringe barrel 250 and a piston 260.

The syringe pump 240 comprises a syringe barrel 250 and a piston 260. The syringe pump heating sleeve 280 is for heating the contents of the syringe barrel 250 as required. While the syringe barrel 250 and the piston 260 are part of the expendable block 120, a linear actuator 270 that drives the syringe pump is part of the reusable block 130.

When the expendable block 120 is inserted into the reusable block 130 and pressed in, a mechanical connection is formed between the linear actuator 270 and the piston 260, allowing for the actuator to be able to pull the piston 260 out by retracting the linear actuator 270 or push the piston 260 in by extending the linear actuator 270, as required, to respectively draw a fluid into the syringe barrel 250 or force the fluid out of the syringe barrel 250.

Still referring to FIG. 3 and FIG. 6, the capillary tube inlet 160 is connected, via the fluidic interconnection 300, to a first fluidic port 320 of the first valve 180. It should be noted that, in the preferred embodiment of the invention, preferably the capillary tube inlet 160 is simply the open end of a capillary tube that constitutes the fluidic interconnection 300 or constitutes a segment of the interconnection 300 that is connected to the first fluidic port 320 of the first valve 180.

A second fluidic port 320 of the first valve 180 is connected to a first fluidic port 320 of the first liquid sensor 210 via the fluidic interconnection 300.

A second fluidic port 320 of the first liquid sensor 210 is connected via a fluidic intersection 310 to a first fluidic port 320 of the second liquid sensor 220 and to a first fluidic port 320 of the third liquid sensor 230.

A second fluidic port 320 of the second liquid sensor 220 is connected to a first fluidic port 320 of the second valve 190 via the fluidic interconnection 300.

A second fluidic port 320 of the second valve 190 is connected to the analysis module inlet 610 of the analysis module 600 via the fluidic interconnection 300.

A second fluidic port 320 of the third liquid sensor 230 is connected to a fluidic port 320 of the syringe pump 240 via the fluidic interconnection 300.

A flow diagram describing one of the many variants of the operation of the assay device 100 is shown in FIG. 7. It will be appreciated that this flow diagram only describes the more significant aspects of only one variant of the operation of the assay device 100. Aspects of the operation of the assay device that can be readily addressed by the skilled worker in the art are also left out of this description, e.g., where the volume of the collected sample reaches the maximum capacity of the assay device.

As described in the following, the operation of the assay device 100 is highly automated, and only a limited degree of user input is required in collecting and analyzing the sample.

After the expendable block 120 is inserted into the reusable block 130 and the connection between the assay device 100 and the host is made, the “control” is transferred to process 2000 through an “interrupt”.

Following the process 2000, the assay device is initialized (process 2010). During the initialization (process 2010) the linear actuator 270 driving the syringe pump 240 is switched to the “off” state and the first valve 180 and the second valve 190 are switched to the “open” state (allowing for the fluid to pass through the valves). After this, the green LED 144 on the assay device's reusable block 130 is turned on, signaling to the user that the procedure for the collection of the sample can commence.

It should be noted that in the preferred embodiment of the invention, the assay device 100 is designed in such a way that, upon the “power-up” of the assay device 100, certain of the “signal lines” of the assay device 100 are “pulled up” or “pulled down”, as required, to put the assay device 100 in the final states that are mandated by the instructions of the process 2010, in a “glitch-free” manner, even before the signals are asserted by the controller as part of the process 2010.

For the NAF sample, the NAF is aspirated according to one of the standard NAF aspiration procedures.

To collect the sample, the user brings the capillary tube inlet 160 of the assay device 100 into contact with the aspirated NAF (or the liquid sample of a different type). The liquid sample is drawn, via capillary action, sequentially through the capillary tube inlet 160, the first valve 180, the first liquid sensor 210, and the second liquid sensor 220.

When the sample reaches the second liquid sensor 220, the presence of the sample is detected by this sensor. This is depicted as process 2020 in the flow diagram.

The detection of the sample by the second liquid sensor 220 is followed by process 2030 where a “first interrupt” is disabled (the reason for this instruction will be clarified in the subsequent processes) and the second valve 190 is closed (preventing the liquid sample from passing through). At this stage the linear actuator 270, driving the piston 260 of the syringe pump 240, is set to retract, drawing the sample into the syringe barrel (i.e., the syringe pump 240 is put in the suction mode).

The syringe pump 240 continues to draw the sample until the first liquid sensor 210 detects a depletion of the liquid sample in the interior of the sensor (process 2040 in the flow diagram). This condition can be due to a situation where the supply of the sample has been exhausted or the contact between the capillary tube inlet 160 and the aspirated NAF sample has been lost. After this condition is detected, the system proceeds by drawing the remnants of the sample that is still present in the flow path in the assay device 100, between the capillary tube inlet 160, the second valve 190, and the third liquid sensor 230, clearing this flow path from the remnants of the liquid sample and making the assay device 100 ready for the collection of any of the sample that may additionally be desired.

This aspect of the assay device operation begins with the green LED 144 getting turned off, indicating to the user to pause the process of the collection of the sample (depicted as process 2050 in the flow diagram). This happens while the syringe pump is still drawing the remnants of the sample that is still present in the flow path in the assay device 100, between the assay device capillary tube inlet 160 and the third liquid sensor 230.

When, as depicted as process 2060 in the flow diagram, the third liquid sensor 230 detects the depletion of the liquid sample in the sensor's interior, process 2070 in the flow diagram is commenced where the linear actuator 270 driving the syringe pump 240 is put in the “off” state, the first valve 180 is closed, the second valve 190 is opened, and then the linear actuator 270 of the syringe pump 240 is retracted to draw any remnants of the sample that would still be present in between the second valve 190 and the fluidic intersection 310 between the flow path that interconnects the first sensor 210, the second sensor 220, and the third sensor 230.

After a suitable period of time delay Δt₁ to assure that the syringe pump 240 draws any remnants of the sample present in between the second valve 190 and the fluidic intersection 310 between the flow path that interconnects the first sensor 210, the second sensor 220, and the third sensor 230, process 2080 in the flow diagram is commenced. Process 2080 turns the linear actuator driving the pump 240 off, enables the first interrupt, and transfers control to process 2010. At this stage, the assay device is reinitialized by rerunning of the process 2010 in the flow diagram and the user can proceed with the collection of more of the sample, as may be desired, after the green LED 144 indicator is turned on once again by the assay device as part of the execution of the process 2010. The next cycle of the collection of the sample is a repetition of what has already been described above. To affect the collection of any additional sample that may be desired, the process 2010 transfers the control to the process 2020. However, as the process 2080 has enabled the first interrupt, an interrupt-driven analysis phase of the sample can be commenced as desired.

Referring back to the process 2030, after enabling the first interrupt during the process 2080, the window for commencing the interrupt-driven analysis phase of the sample will last until the disabling of the first interrupt during the execution of the process 2030. However, when it is desired to commence the interrupt-driven analysis phase of the sample, naturally no more of the sample would be collected and the control would stay within the process 2020 and will not reach the instruction in the process 2030 that disables the first interrupt.

After the desired amount of the liquid sample has been collected, the analysis phase can commence. The analysis phase of the assay device operation begins by the user giving the appropriate “analyze” command to the assay device. This command can be given by momentarily depressing and releasing the tactile switch 150 on the assay device or via a command being initiated via the user interface of the application software on the host. The analyze command is interrupt-driven, and it interrupts the control flow, transferring control to process 2090 in the flow diagram.

The analysis of the sample commences according to process 2110 in the flow diagram where the green LED 144 is turned off, indicating to the user that no more of the sample is to be collected, the red LED 142 is turned on, as the indication that the analysis phase is in progress, and the first valve 180 and the second valve 190 are closed.

The syringe barrel 250 may optionally also store one or more assay reagents 840. The assay reagents are loaded in the syringe barrel 250 during the assembly of the assay device expendable block 120. In the preferred embodiments of the invention, the assay reagents are stored in the syringe barrel 250 in a dry form.

At this stage, the liquid sample that had been drawn into the syringe barrel 250 has undergone some mixing with the assay reagents that had been stored in the syringe barrel 250. The resulting mix will be referred to as a “chemical mix” or a “biochemical mix” for the purpose of this patent application, and the resultant fluid will be referred to as the chemical mix or the biochemical mix irrespective of whether the assay reagents 840 are stored in the syringe barrel 250 or not and also irrespective of any subsequent processing that the chemical mix or the biochemical mix may still undergo.

The biochemical mix now undergoes a number of physical and biochemical processes (depicted as process 2120 in the flow diagram). These processes take place by appropriately heating the biochemical mix inside the syringe barrel 250 via the heating sleeve 280 that surrounds the syringe barrel 250. In effect, the syringe barrel 250 additionally functions as a reaction chamber.

The physical and biochemical processes that take place in the reaction chamber (syringe barrel 250, in this embodiment) include the mixing of the liquid sample with the assay reagents, the lysis of any cells in the biochemical mix to release their genetic material, and the amplification of various desired segments of this genetic material. The genetic material includes one or more of DNA molecules, RNA molecules, and their methylated forms. The biochemical mix wherein the genetic material in the biochemical mix has undergone amplification is called the amplicon.

The physical and biochemical processing of the biochemical mix is a timed process. After the amplification of the genetic material in the biochemical mix has been completed, the second valve 190 is opened and the linear actuator 270 driving the pump 240 is set to extend to force the biochemical mix out of the syringe barrel 250 and, through the analysis module inlet 610, into the analysis module 600 (depicted as process 2130 in the flow diagram). Enough of the biochemical mix is forced into the analysis module 600 to fill the detection chambers 650 that are incorporated in the analysis module 600. The method by which the filling of the detection chambers 650 of the analysis module 600 is determined relies on one or more of the liquid sensors 200 that are located in the flow path in the analysis module 600 in such a way as to be able to ascertain the filled state of the detection chambers 650 in the analysis module 600. After a suitable time interval after the liquid sensors 200 detect the presence of liquid in the “sensors' manifolds”, it can be assumed that all of the detection chambers 650 are filled; therefore, the syringe pump 240 continues to force the biochemical mix into the detection chambers 650 of the analysis module 600 until after the liquid sensors 200 have indicated the presence of the liquid and after the suitable time interval after the detection of the liquid has elapsed (depicted as process 2890 in the flow diagram).

At this stage, as depicted as process 2150 in the flow diagram, the actuator 270 driving the syringe pump 240 is turned off and the detection of the various target molecules (target nucleic acid sequences or various target proteins), contained in the biochemical mix, can now commence.

Each of the detection chambers 650 in the analysis module 600 includes a probe specific to a particular analyte. The analyte may be a nucleic acid sequence. Each of the detection chambers 650 includes an electrochemiluminescence resonance energy transfer probe that is anchored to an interior surface of the detection chamber 650. For the purpose of this patent application, the chemical mix or the biochemical mix forced into the detection chambers 650 and the electrochemiluminescence resonance energy transfer probe will together be referred to as the contents of the detection chambers 650. The analysis module 600 comprises one or more of heaters 680 for heating the contents of the detection chambers 650. When a nucleic acid sequence in the biochemical mix is complementary to the electrochemiluminescence resonance energy transfer probe in a detection chamber 650, the nucleic acid sequence hybridizes with the electrochemiluminescence resonance energy transfer probe and forms a hybrid, indicating the presence of a target molecule (a target nucleic acid sequence).

Electrical excitation of the content of the detection chambers 650 in parallel to the detection of any emission by the electrochemiluminescence resonance energy transfer probes in the detection chambers is used to detect the existence of hybrids in each of the detection chambers 650.

Each of the detection chambers 650 incorporate two or more excitation electrodes 690, an anode and a cathode. Additionally, the analysis chip comprises one or more photosensors 710 adjacent to the detection chambers 650 for detecting any emission by the electrochemiluminescence resonance energy transfer probes in the detection chambers 650. An excitation current is forced between the two electrodes 690 in each of the detection chambers 650, and any electrochemiluminescence emission by the electrochemiluminescence resonance energy transfer probe is detected by the photosensors 710 adjacent to the detection chambers 650. Under this arrangement, the detection of the emission by the electrochemiluminescence resonance energy transfer probes in the detection chambers 650 corresponds to the detection of the nucleic acid sequences complementary to the probes that have been loaded in the detection chambers.

The hybridization data gathered, in the manner described, is further processed by the host and compared to the database of the diagnostic markers (e.g., disease markers) and/or prognostic markers to meet the diagnostic and prognostic objectives.

At this stage, with the detection of the presence of the diagnostic markers and/or prognostic markers having been completed, the host turns the red LED 142 off and the blue LED 146 on, indicating to the user that the analysis results are ready. This is depicted as process 2170 in the flow diagram. In parallel to the indication given to the user by the LEDs, the user interface of the application software running on the host also provides an indication to the user of the completion of the detection and analysis phase.

At this point, the user can use the user interface of the application software on the host to view or further process the results of the analysis of the sample.

After the completion of the process 2170, process 2180 returns the control to higher-level host software.

At this stage, the user may remove the expendable block 120 of the assay device 100 and dispose of it accordingly. Removing the expendable block 120 triggers an interrupt, which transfers the control to process 2190, which in turn transfer control to process 2200.

As indicated in the flow diagram, during the execution of the process 2200, the blue LED 146 is turned off, the red LED 142 is turned on, the linear actuator 270 that drives the piston of the syringe pump 240 is set to extend, and after a time delay of Δt₂, the linear actuator 270 that drives the piston of the syringe pump 240 is set to the off state. The time delay Δt₂ is determined by the application software in such a way that the linear actuator 270 that drives the piston of the syringe pump 240 is suitably extended to make the reusable block 130 of the assay device 100 ready for receiving an unused unit of the expendable block 120 for another analysis cycle, if desired. Then, as indicated in process 2210, the control is returned to higher-level application software, and the diagnostic, prognostic, and analytical system waits for any desired further use.

In the embodiment just described, the process 2200 is a “timed” process, where the time delay Δt₂ is determined by the application software in such a way that the linear actuator 270 is suitably extended to make the reusable block 130 of the assay device 100 ready for receiving an unused unit of the expendable block 120. In other embodiments, the process 2200 is not a timed process, and instead other techniques are used in assuring that the linear actuator 270 is suitably extended to make the reusable block 130 of the assay device 100 ready for receiving an unused unit of the expendable block 120. In one embodiment, the technique that is used to assure that the linear actuator 270 is suitably extended to make the reusable block 130 of the assay device 100 ready for receiving an unused unit of the expendable block 120 comprises a “positional sensor” that is “linked” to the linear actuator that drives the piston of the first syringe pump 240. The output of the positional sensor is used to provide feedback to suitably control the linear actuator 270 in such a way that the linear actuator 270 is suitably extended to make the reusable block 130 of the assay device 100 ready for receiving an unused unit of the expendable block 120.

### The Analysis Module and the Analysis Chip

As mentioned previously, the assay device 100 incorporates the analysis chip 602. The analysis chip 602 is one of the analysis chip embodiments. The analysis chip 602 incorporates an embodiment of the analysis module 600. The analysis module 600 can perform a wide variety of fluidic, electronic, optoelectronic, and biochemical functions. In particular, the analysis module 600 module is configured to detect the presence or absence of at least an analyte in the sample. The analyte may be a diagnostic marker (e.g., a disease marker) and/or a prognostic marker. In some examples, the analysis module may be considered what is known in the art as a micro total analysis system module (μTAS module) or the like. In this section a more detailed description of the analysis module 600 and the analysis chip 602 is given.

As it is difficult to distinguish the details in the layout design of the analysis module 600 and the analysis chip 602 as shown in FIG. 4, the analysis module 600 and the analysis chip 602 are described using diagrams that depict the various aspects of the design in a simplified layout form.

FIG. 8 shows a simplified layout of the analysis chip 602. The analysis chip 602 has a chip boundary 608. The analysis module 600 incorporates all of the components of the analysis chip 602 that are inside the chip boundary 608 of the analysis chip 602. In this embodiment of the analysis chip 602, the components incorporated in the analysis module 600 are fabricated on the substrate 409 (see FIG. 31).

FIG. 9 shows more details of the detection chamber 650, and FIG. 10 shows a cross-section of the detection chamber 650.

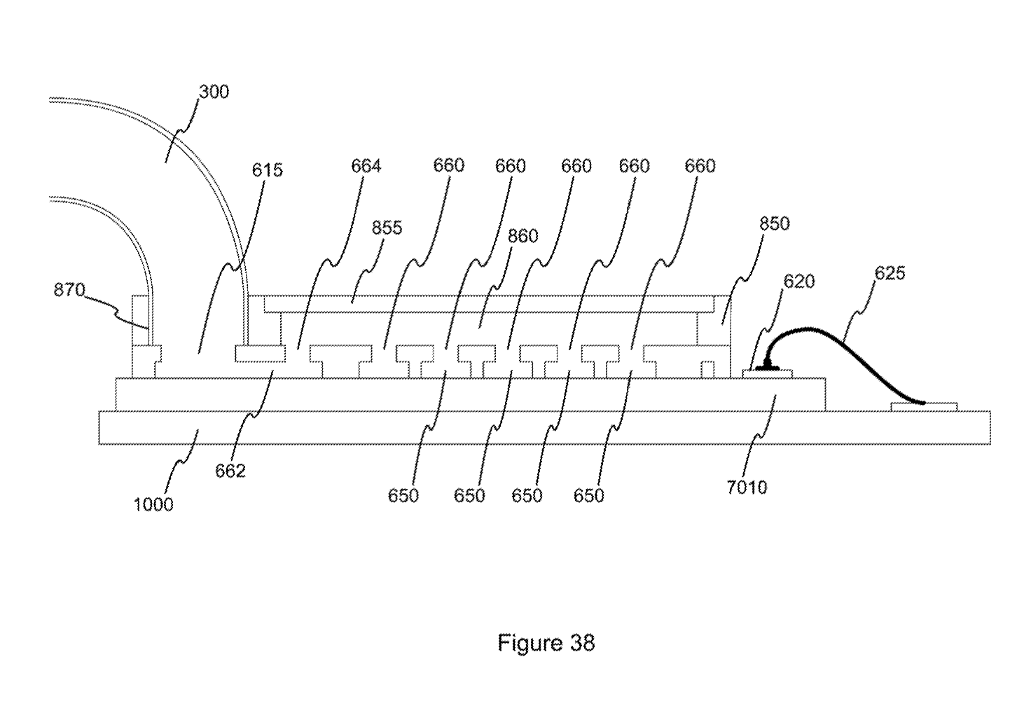

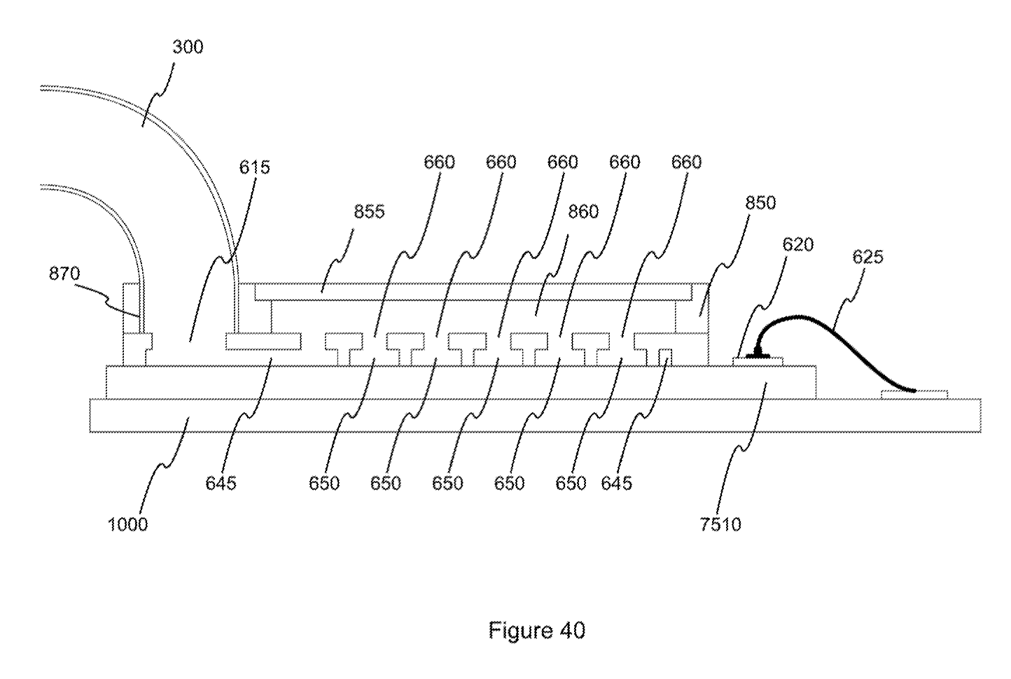

The bond pads 620 are used to connect the analysis module 600 to the rest of the system via bond wires 625 (see FIG. 38 and FIG. 40 for other embodiments of the analysis chip). The roles of these connections comprise receiving electrical power from the rest of the system, receiving data and/or other signals from the rest of the system, and transmitting data and/or other signals to the rest of the system.

The chemical mix or the biochemical mix is fed via the analysis module inlet 610 into the analysis chip. The chemical mix or the biochemical mix is split into a first branch 630 and a second branch 640 of a fluidic network of channels 645. The second branch 640 optionally incorporates various sensors (not shown) that can perform various characterizations of the chemical mix or the biochemical mix. The first branch 630 leads into an array incorporating the detection chambers 650, feeding the chemical mix or the biochemical mix into each of the detection chambers 650 through a detection chamber inlet 655.

Each detection chamber 650 has a roof opening 660 in its roof. The role of the roof opening 660 is multifold. The roof opening 660 provides access to the interior of the detection chamber 650, allowing the loading of each of the detection chambers 650 with probes or other chemicals during the assembly of the assay device, and the roof opening 660 provides a means for air to escape the detection chambers 650 and the first branch 630 of fluidic channels in front of the chemical mix or the biochemical mix as the chemical mix or the biochemical mix moves in to fill the detection chambers 650.

As shown in FIG. 8, the analysis module 600 also incorporates one or more of calibration and/or control chambers 670 that are similar to the detection chambers 650 except that the calibration and control chambers 670 are isolated from the fluidic channels in the first branch 630 and, as such, would not be fed with the chemical mix or the biochemical mix during the assay process. The calibration and/or control chambers 670 are used for calibration and/or control purposes. The calibration and/or control chambers 670 are used, as required, with probes or other chemicals being loaded in them during assembly or with empty chambers.

The analysis module 600 incorporates one or more heaters 680 for controlling the temperature of the chemical mix or the biochemical mix during the various stages of operation of the analysis module 600. The heaters 680 are resistive heaters that are heated by an electrical current being forced through them. By controlling the current in a suitable manner, the temperature of the mix can be controlled. The sensors and the power and control circuitry for providing power to the heaters 680 and controlling the heaters 680 are not shown.

Additional details of the detection chamber 650 are shown in FIG. 9 and FIG. 10. As can be seen in these figures, the photosensor 710 is adjacent to the detection chamber 650. The photosensor 710 incorporates a photodiode or other photodetector; it also incorporates additional analog and digital circuitry (not shown). The photosensor 710 provides for the detection of any electrochemiluminescence emission of photons by the electrochemiluminescence resonance energy transfer probes in the detection chambers 650, as was described earlier in this specification.

Electrodes 690, shown in FIG. 9 and FIG. 10, are for exciting the electrochemiluminescence resonance energy transfer probes. One of the electrodes is the anode, and the other one is the cathode. The excitation current is forced by the electronics incorporated in the analysis module 600 through these two electrodes.

The embodiment of the analysis chip 600 that is shown in FIG. 4 and FIG. 8 includes an array of 96 detection chambers 650, a network of resistive heaters 680, one or more of the liquid sensors 200, an array of four-point resistance temperature detectors (not shown), and an array of electrode-pairs (not shown) for liquid-conductivity measurement.

The skilled worker will appreciate that the geometric locations of many of the components of the analysis module 600 or the analysis chip 602 can be varied without changing the functionality of the analysis module 600 or the analysis chip 602; this implies that the length of electrical interconnections (not shown) or the fluidic interconnections can be varied by the skilled worker in such a way that does not change the basic functionality of the analysis module 600 or the analysis chip 602.

The skilled worker will also appreciate that the quantitative aspects of many of the components of the analysis module 600 or the analysis chip 602 can be varied to scale the capabilities of the analysis module 600 or the analysis chip 602 without changing the basic role of the analysis module 600 or the analysis chip 602, e.g., the number of the detection chambers 650 of the analysis module 600 can be increased by the skilled worker to scale up the number of the probes the analysis module 600 can incorporate.

### Flow Path

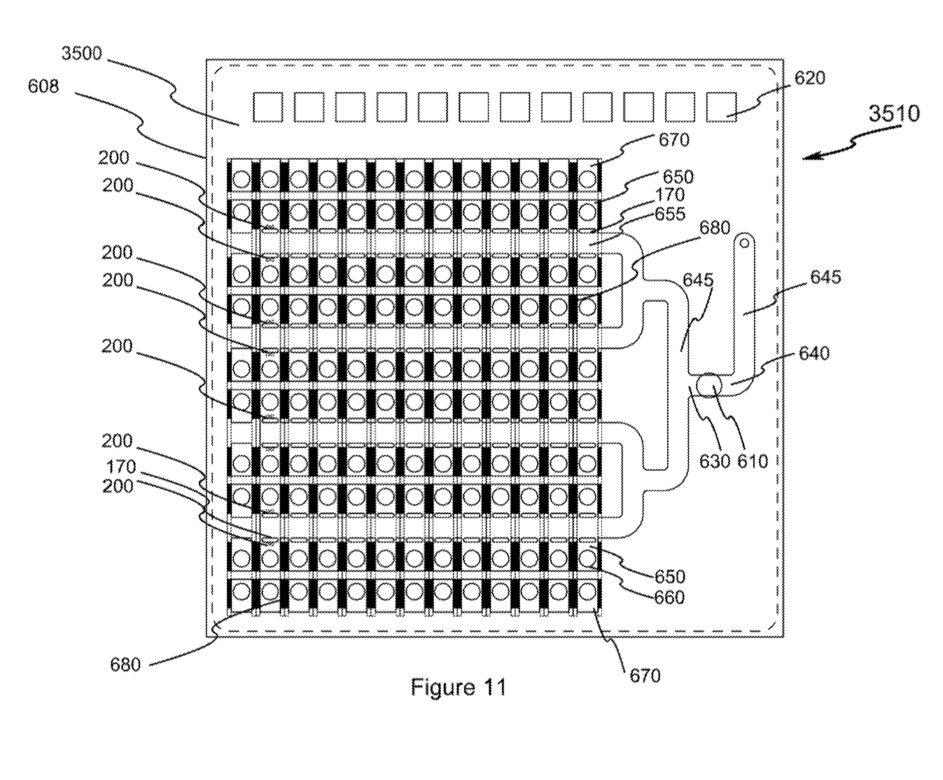

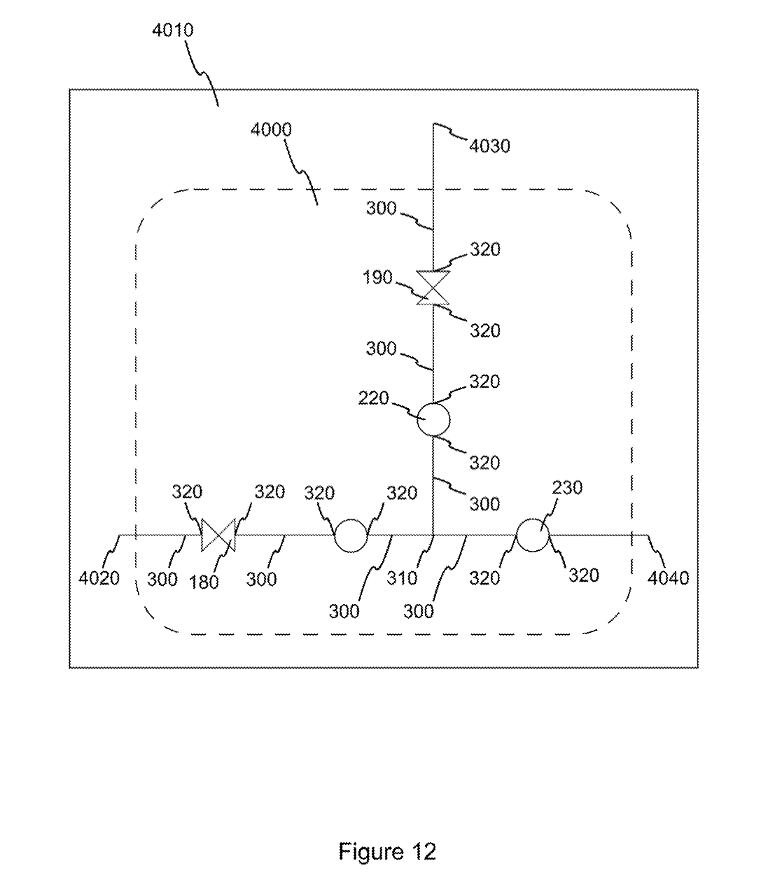

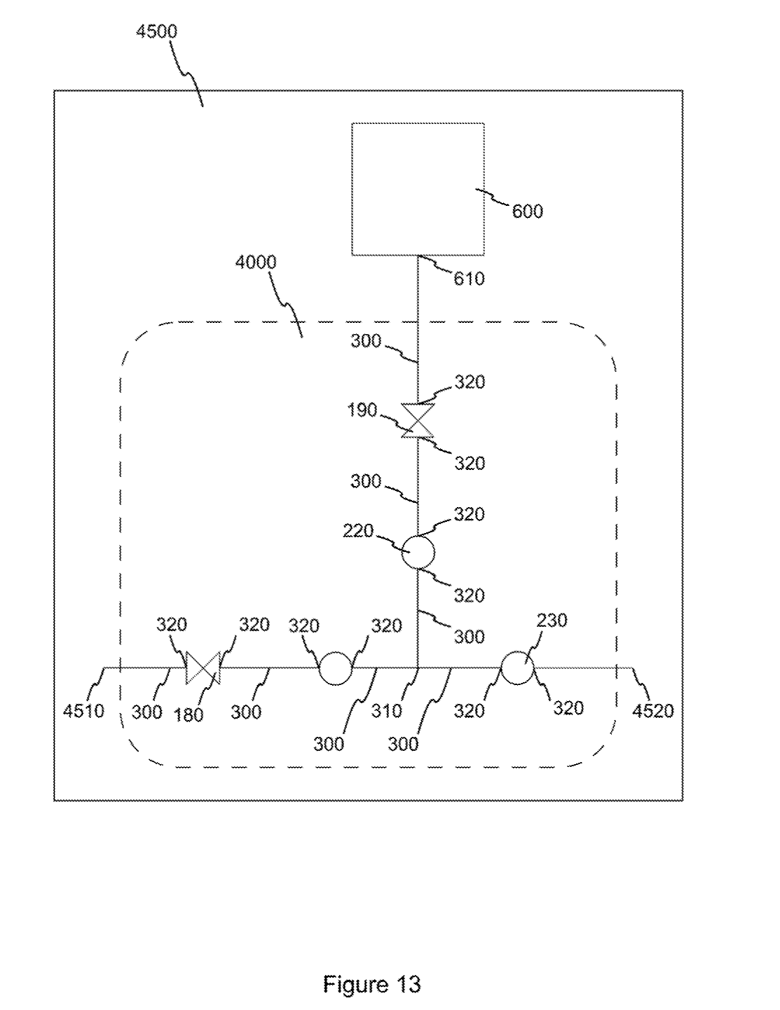

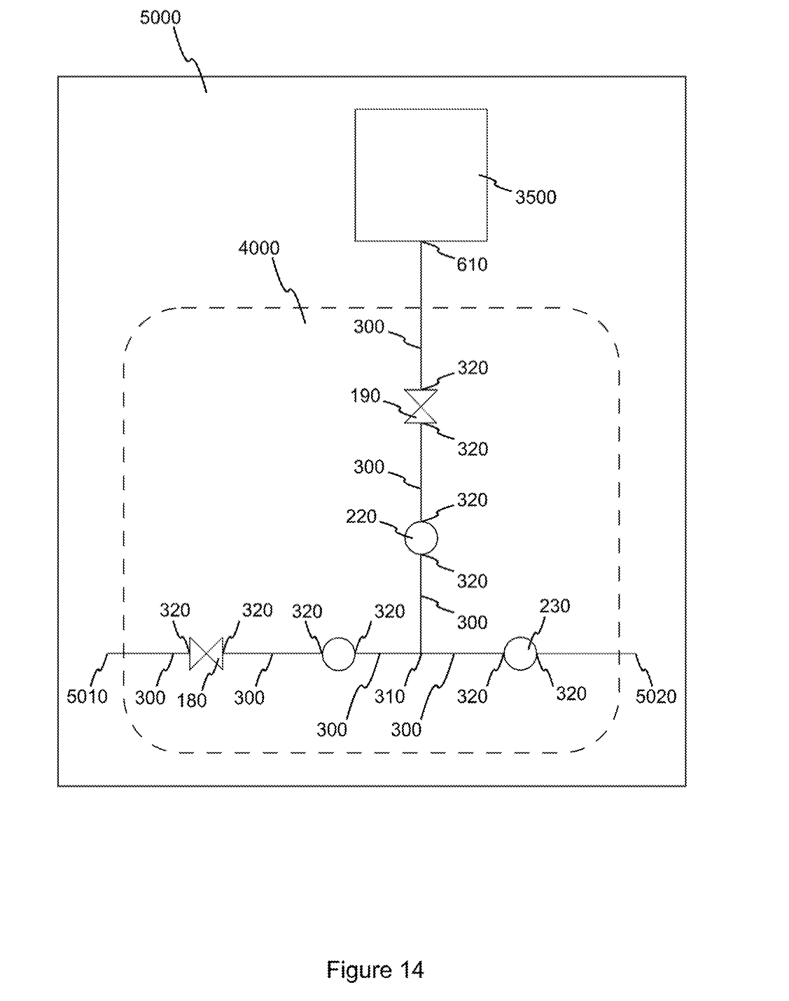

Referring, for example, to FIG. 6, FIG. 8, FIG. 9, FIG. 11, FIG. 12, FIG. 13, and FIG. 14, any combination of the capillary tube inlet 160, a first valve 180, a second valve 190, a first liquid sensor 210, a second liquid sensor 220, a third liquid sensor 230, a syringe pump 240, an analysis module 600, the fluidic interconnections 300 between these components, analysis module inlet 610, first branch 630, second branch 640, channels 645, detection chambers 650, or any other component or feature that at times permits the flow of a liquid from at least one of these components to at least another one of these components is considered to constitute a flow path for the purpose of this patent application. For example, the flow paths may be provided via channels, microchannels, tubing, analysis module, or other like physical structures present in the assay device, or combinations thereof.

### Other Embodiments of the Invention

Additionally to the embodiments described above other embodiments of the invention will now be described, some only in schematic form. In the interest of clarity, in some cases, some of the components such as liquid sensors and temperature sensors have been omitted from the illustrations or the descriptions, but it will be appreciated that these have been incorporated accordingly, and in appropriate places, in each of the following embodiments.

### A Second Embodiment of Analysis Module

An analysis module 3500 will be described next. The analysis module 3500 is a second embodiment of the analysis module, and it is depicted in FIG. 11 in a simplified layout form and will be described next.

In the preferred embodiment of the invention, the analysis module 3500 is incorporated in an analysis chip 3510. The analysis chip 3510 is fabricated on a substrate 409 (see FIG. 31) using microfabrication process steps that will be described later in this specification.

The analysis module 3500 is identical to the analysis module 600 with the exception that it incorporates a valve 170 at the detection chamber inlet 655 of each of the detection chambers 650. Before the beginning of process steps depicted as process 2130 in the flow diagram, the valves 170 are open, allowing the mix to flow into the detection chambers 650. After the filling of the chambers 650, the valves 170 are closed. The functionality improvement provided by the incorporation of the valves 170 in the analysis module 3500 is that the analysis module 3500 can utilize unanchored probes or chemicals in its detection chambers 650, with the valves 170 preventing the backflow and the escape of the unanchored chemicals out of the detection chambers 650. This functionality improvement, provided via the incorporation of the valves 170 in the analysis module 3500, caters for a better assay signal-to-noise ratio, an increase in the reliability of the assay process, and a simpler probe design.

Additionally, in some embodiments of the invention, the detection chambers 650 of the analysis module 3500 are optionally loaded with the primers and other chemicals and biochemicals necessary for amplification of nucleic acids. In these embodiments, with the valves 170 providing isolation between the detection chambers 650, each of the detection chambers 650 is capable of optionally performing a simplex or multiplex nucleic acid amplification reaction separate from the rest of the detection chambers 650, enabling the analysis module with the capability to perform parallel amplification of nucleic acids. A number of embodiments of the assay device that incorporate the parallel nucleic acid amplification capability use this capability of the analysis module to affect parallel amplification of nucleic acids in lieu of or in tandem to any up-stream reaction that takes place in the assay device.

Other embodiments of the analysis module will be described later in this specification that also incorporate a valve 170 at the detection chamber inlet 655 of each of the detection chambers 650 and, hence, provide the same capabilities as those described above.

A first Embodiment of Integration Chip

An integration chip integrates the functionality of a number of components of the assay device 100 in a single device. As such, an integration chip can be substituted for a number of components of the assay device 100.

An integration chip 4010 will be described next. The integration chip 4010 is a first embodiment of the integration chip, and it is depicted in FIG. 12 in schematic form and will be described next.

The integration chip 4010 incorporates a first embodiment of an integration module 4000.

The integration module 4000 incorporates integrated fluidic, electromechanical, and electronic subsystems. Preferably the integration chip 4010 is fabricated using microfabrication process steps that include various process steps that are also used for the fabrication of the analysis chip 602. These process steps are described later in this specification.

The integration chip 4010 incorporates a first fluidic port 4020, a second fluidic port 4030, a third fluidic port 4040, a first valve 180, a second valve 190, a first liquid sensor 210, a second liquid sensor 220, a third liquid sensor 230, and the fluidic interconnections 300. All of these components are integrated on a substrate 409 (see FIG. 31).

As illustrated in FIG. 12, the components in the integration chip 4010 and the interconnectivity of the said components with each other replicate the module 105 of the assay device 100 (see FIG. 6). This means that the module 105 of the assay device 100 can be replaced with the integration chip 4010, with the full functionality of assay device 100 maintained, where the functionality of the first fluidic port 4020 of the integration chip 4010 is identical to the first fluidic port 320 of the first valve 180 of the assay device 100, the functionality of the second fluidic port 4030 of the integration chip 4010 is identical to the second fluidic port 320 of the second valve 190 of the assay device 100, and the functionality of the third fluidic port 4040 of the integration chip 4010 is identical to the second fluidic port 320 of the third liquid sensor 230 of the assay device 100.

Still referring to FIG. 12, to recap, the first fluidic port 4020 of the integration chip 4010 is connected, via fluidic interconnection 300, to a first fluidic port 320 of the first valve 180. It should be noted that, in the preferred embodiment of the invention, the first fluidic port 4020 of the integration chip 4010 is simply the first fluidic port 320 of the first valve 180.

A second fluidic port 320 of the first valve 180 is connected to a first fluidic port 320 of the first liquid sensor 210 via fluidic interconnection 300.

A second fluidic port 320 of the first liquid sensor 210 is connected via a fluidic intersection 310 to a first fluidic port 320 of the second liquid sensor 220 and to a first fluidic port 320 of the third liquid sensor 230.

A second fluidic port 320 of the second liquid sensor 220 is connected to a first fluidic port 320 of the second valve 190 via the fluidic interconnection 300.

A second fluidic port 320 of the second valve 190 is connected to the second fluidic port 4030 of the integration chip 4010 via the fluidic interconnection 300.

A second fluidic port 320 of the third liquid sensor 230 is connected to the third fluidic port 4040 of the integration chip 4010 via the fluidic interconnection 300.

It should be noted that, in the preferred embodiment of the invention, the second fluidic port 4030 of the integration chip 4010 is simply the second fluidic port 320 of the second valve 190, and the third fluidic port 4040 of the integration chip 4010 is simply the second fluidic port 320 of the third liquid sensor 230.

A third Embodiment of Analysis Chip

An analysis chip 4500 will be described next. The analysis chip 4500 is a third embodiment of the analysis chip, and it is depicted in FIG. 13 in schematic form and will be described next.

The third embodiment of the analysis chip 4500 integrates the analysis module 600, the integration module 4000, a first fluidic port 4510, a second fluidic port 4520, and the fluidic interconnections 300 on one substrate 409 (see FIG. 31).

The analysis chip 4500 is fabricated using microfabrication process steps that are a superset of the process steps that are used for fabrication of the analysis chip 602. These process steps are described later in this specification.

### A Fourth Embodiment of Analysis Chip

An analysis chip 5000 will be described next. The analysis chip 5000 is a fourth embodiment of the analysis chip, and it is depicted in FIG. 14 in schematic form and will be described next.

The fourth embodiment of the analysis chip 5000 integrates the analysis module 3500, the integration module 4000, a first fluidic port 5010, a second fluidic port 5020, and the fluidic interconnections 300 on one substrate 409 (see FIG. 31).

The analysis chip 5000 is fabricated using microfabrication process steps that are a superset of the process steps that are used for fabrication of the analysis chip 602. These process steps are described later in this specification.

### Hydrogel Valve

Some of the embodiments the analysis modules and the assay devices optionally employ temperature-responsive hydrogel valves to control fluid flow.

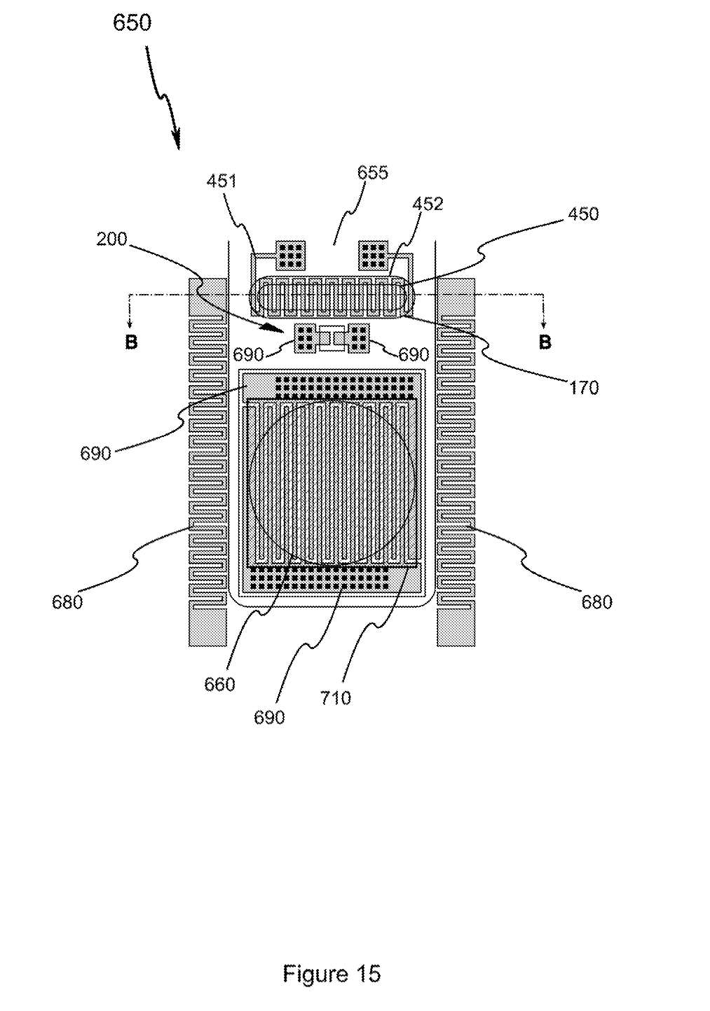

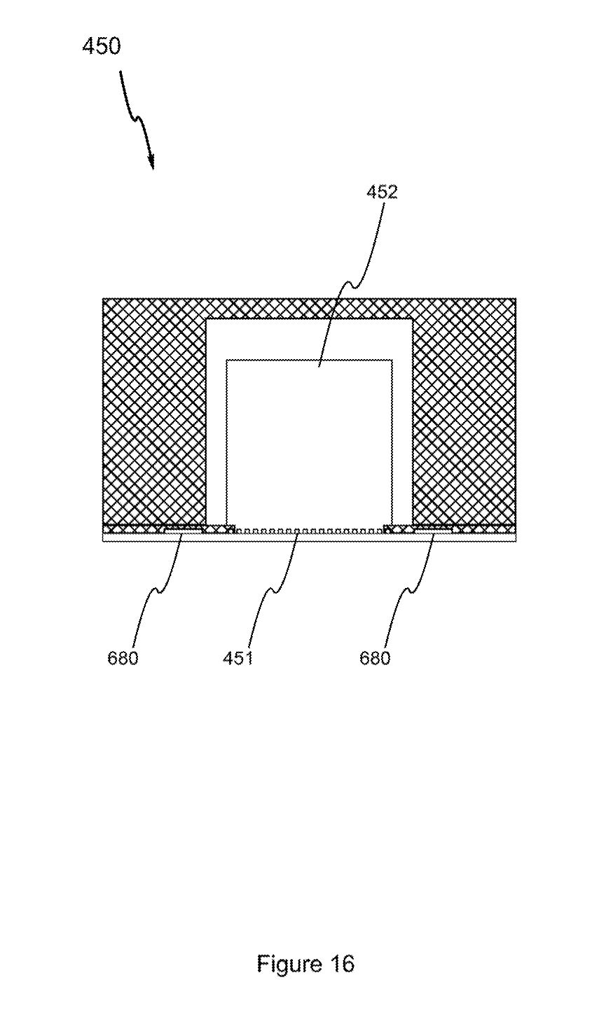

See FIG. 11, FIG. 15, and FIG. 16. In the embodiment of the second variant of the analysis module 3500 shown in FIG. 11, each of the valves 170 is a hydrogel valve 450, which will be described next.

FIG. 15 shows the layout of the detection chamber 650 of the type used in the analysis module 3500. The figure additionally shows the valve 170 at the detection chamber inlet 655. The valve 170 shown is a hydrogel valve 450, comprising a hydrogel valve heater 451, and a hydrogel valve actuator 452. Cross-section B, as defined in FIG. 15, of the detection chamber 650 of the type used in the analysis module 3500 is shown in FIG. 16.

FIG. 15 and FIG. 16 show the hydrogel valve 450 in its open state. In the open state of the hydrogel valve 450, the detection chamber inlet 655 is only partially occluded by the hydrogel valve actuator 452, allowing for the chemical mix or the biochemical mix to flow through the detection chamber inlet 655, past the hydrogel valve actuator 452.

To switch the hydrogel valve 450 from its closed state to its open state or to maintain the valve in its open state, a suitably large current is forced through the hydrogel valve heater 451 to heat the hydrogel valve heater 451 and to subsequently heat the hydrogel valve actuator 452 to a temperature above the lower critical solution temperature (LCST) of the hydrogel valve actuator 452. Heating of the hydrogel valve actuator 452, to a temperature above the LCST of the hydrogel valve actuator 452, results in the contraction of the hydrogel valve actuator 452 or results in the hydrogel valve actuator 452 maintaining its contracted state. As explained above, the contracted state of the hydrogel valve actuator 452 corresponds to the open state of the hydrogel valve 450.

To switch the hydrogel valve 450 to its closed state, the hydrogel valve actuator 452 is to be wetted, with the temperature of the hydrogel valve actuator 452 maintained below the LCST of the hydrogel valve actuator 452. As the chemical mix or the biochemical mix flows through the detection chamber inlet 655 and past the hydrogel valve actuator 452, and with no electric current flowing through the hydrogel valve heater 451, the hydrogel valve actuator 452 is wetted, leading to the swelling of the hydrogel valve actuator 452 to such an extent to completely occlude the detection chamber inlet 655 and to close the hydrogel valve 450.

The hydrogel valves 450 in some of the embodiments of the invention use a copolymer based on polyisopropylacrylamide (polyNIPAAm) (Zhai, 2013). PolyNIPAAm is in a hydrophilic state below its LCST of 32° C. and is in a hydrophobic state above LCST of 32° C. In other embodiments, it is desirable to increase the LCST in line with the operating temperatures of the assay device and of the physical and the biochemical processes taking place in the assay device. In these embodiments the properties of the hydrogel are readily tuned by varying the monomer structure and altering the ratio of hydrophilic to hydrophobic monomers in the copolymer to vary the LCST (Pena-Francesch, A.; Montero, L.; Borrós, S. Tailoring the LCST of thermosensitive hydrogel thin films deposited by iCVD, Langmuir 2014, 30, 7162-7167) and by introducing microstructures within the hydrogel to decrease the switching time (Cai, W.; Gupta, R. B. Poly(N-ethylacrylamide) hydrogels for lignin separation, Ind. Eng. Chem. Res. 2001, 40, 3406-3412). For example, high LCSTs is achieved by increasing the hydrophilic content of the copolymers. Poly[2-(dimethylamino)ethyl acrylate-co-N,N-dimethylacrylamide] [poly(DMAEE-co-NNDMAAm] at a monomer feed ratio of 2:1 has an LCST of 78° C. and at a feed ratio of 1:2 has an LCST of 58° C. (Pooley, S. A.; Rivas, B. L.; Pizarro, G. D. C. Hydrogels based on (dimethylamino)ethylacrylate (DMAEA) and N,N-dimethylacrylamide (NNDMAAm): synthesis, characterization, and swelling behavior, J. Chil. Chem. Soc. 2013, 58, 1597-1602).

As had been described previously, the use of the hydrogel valve 450 in the analysis module 3500 does not require for the hydrogel valve 450 to be opened during normal use.

### Analysis Chip Fabrication

The analysis chip 602, the analysis chip 3510, the analysis chip 7010 (to be described later in this specification), the analysis chip 7510 (to be described later in this specification), and many other embodiments of the analysis chip are preferably fabricated using a microfabrication process. In the description of the microfabrication process that will follow, all of the above embodiments of the analysis chip will be referred to as the “analysis chip” without using any reference numbers.

The microfabrication process used includes a plurality of microfabrication process phases for realizing the fluidic, mechanical, electronic, and optoelectronic subsystems incorporated in the analysis chip.

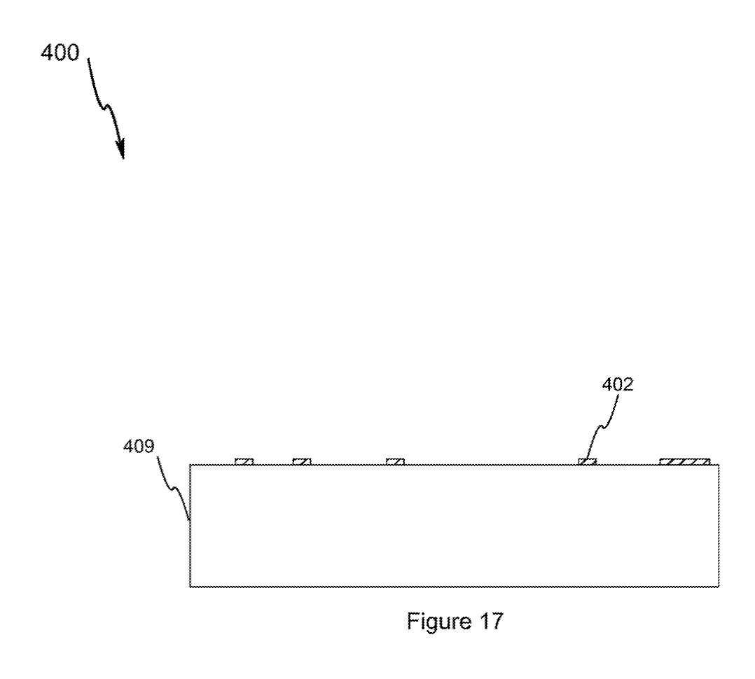

During the first phase of the microfabrication process, a base wafer 400 (shown in FIG. 17) incorporating the electronic and optoelectronic subsystems of the analysis chip is preferably fabricated starting with a standard blank wafer, preferably using a truncated variant of a standard microelectronic fabrication process. The truncated variant of the standard microelectronic fabrication process will be described further down in this specification.

During the second phase of the microfabrication process, the fluidic and the mechanical subsystems are fabricated using a plurality of micromachining techniques.

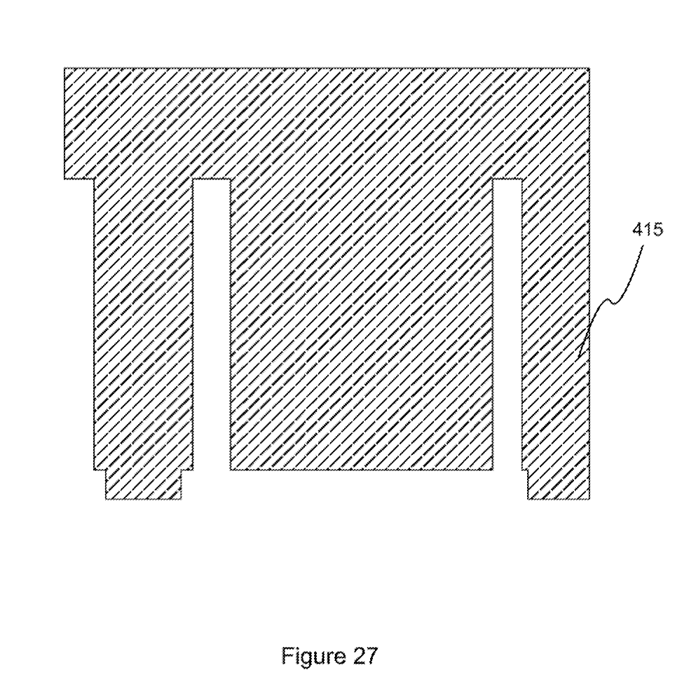

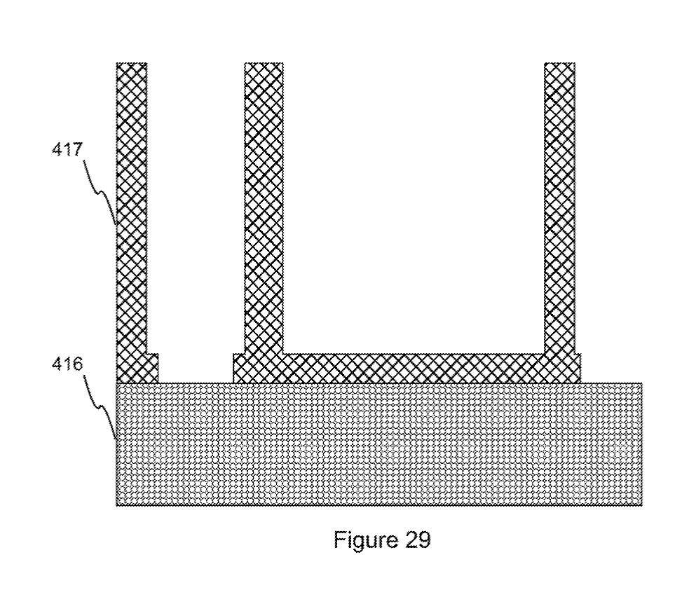

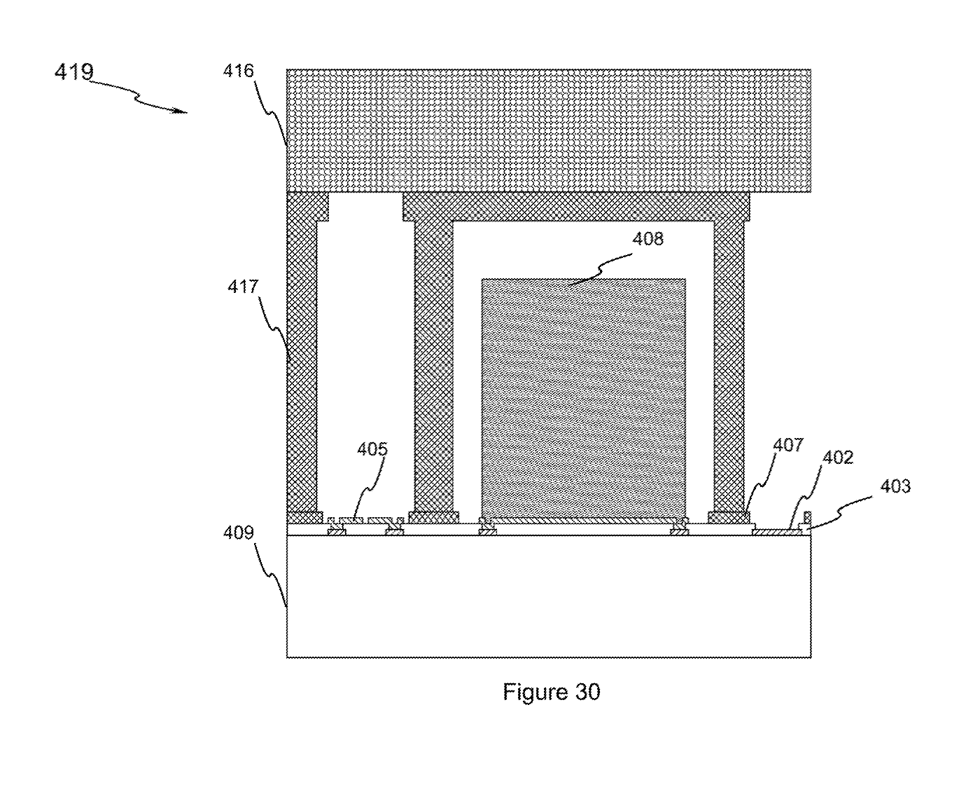

The second phase of the microfabrication process incorporates five stages that include the surface micromachining of the base wafer 400 subsequently to the fabrication of the electronic and the optoelectronic subsystems during the first phase of the microfabrication process, the microfabrication of a stamp 415 (shown in FIG. 27) for a chip cap 417 (best shown in FIG. 29, FIG. 30, and FIG. 31), the fabrication of the chip cap 417 using the stamp 415 previously fabricated, the bonding of the chip cap 417 to the base wafer 400, and the singulation of the analysis chips.

The first phase of the microfabrication process preferably begins with a silicon wafer as the substrate 409 for the fabrication of the base wafer 400. As previously stated, all of the electronic and optoelectronic subsystems are preferably fabricated using the truncated variant of the standard microelectronic fabrication process.

The standard microelectronic fabrication process is preferably a standard CMOS fabrication process. The truncated variant of the standard microelectronic fabrication process incorporates the steps of the standard CMOS fabrication process up to the completion of the patterning of the top metal layer 402. See FIG. 17 for a schematic cross-sectional depiction of the base wafer 400 at the end of the first phase of the microfabrication process.

After the completion of the first phase of the microfabrication process, the first stage of the second phase of the microfabrication process commences. As previously stated the first stage of the second phase of the microfabrication process includes the surface micromachining of the base wafer 400.

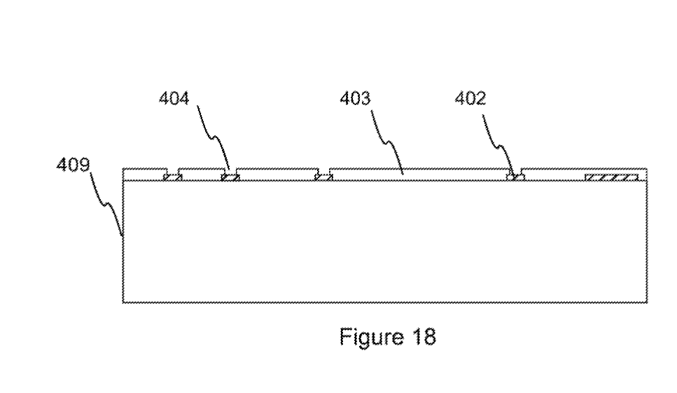

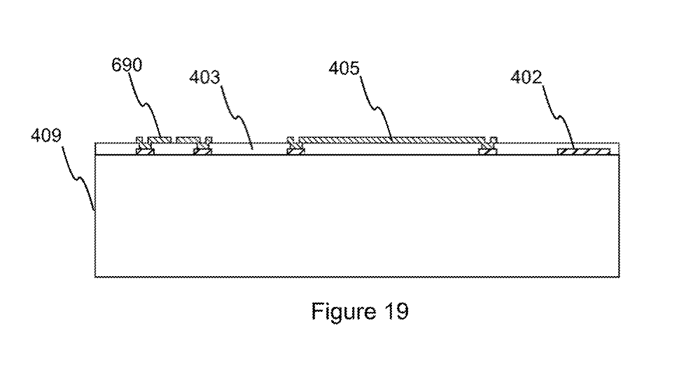

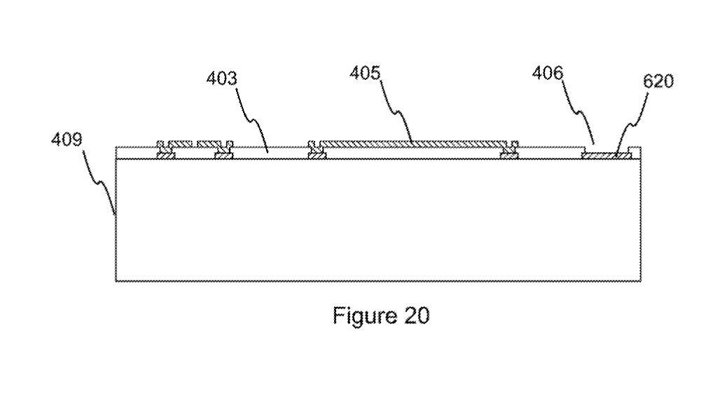

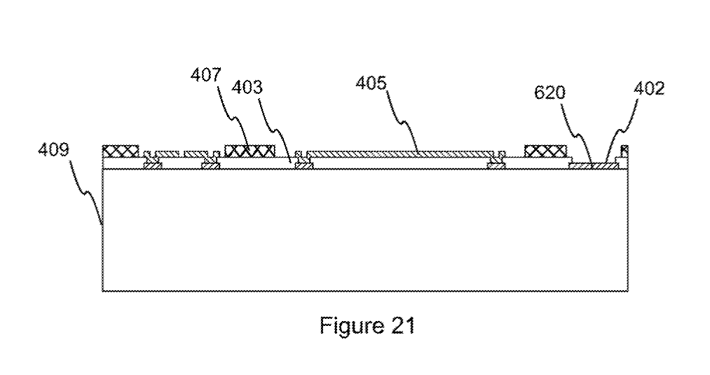

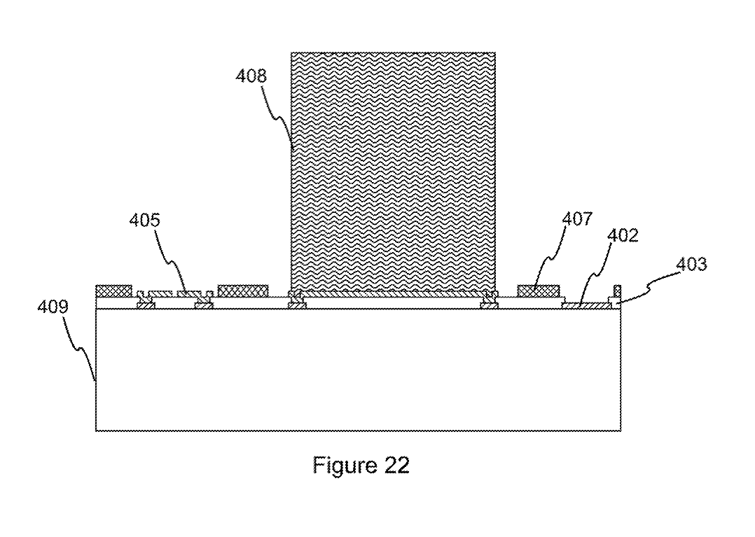

Referring to FIG. 17, FIG. 18, FIG. 19, FIG. 20, FIG. 21, and FIG. 22, the first stage of the second phase of the microfabrication process will be described next.

As depicted in FIG. 18, a passivation layer 403 is deposited on the base wafer 400. The passivation layer 403 preferably comprises a 1 μm “stack” of silicon dioxide, silicon nitride, and silicon dioxide layers. The passivation layer 403 is preferably then planarized using, for example, a standard chemical mechanical planarization process (CMP). The passivation layer 403 is patterned to open a plurality of contact holes 404 in the passivation layer 403. The contact holes 404 provide openings for creating electrical contacts between the top metal layer 402 and the electrically active layers that follow the first phase of the microfabrication process.

As depicted in FIG. 19, a first transducer layer 405 is deposited on the base wafer 400. In the preferred embodiment of the invention, the first transducer layer 405 preferably comprises a 1 μm layer of platinum. The first transducer layer 405 is then patterned to create structures, which include a plurality of heaters 680 and a plurality of electrodes 690 (see for example FIG. 15). The first transducer layer 405 makes contact with the top metal layer 402 through the contact holes 404.

As depicted in FIG. 20, the passivation layer 403 is again patterned to open a plurality of bond pad openings 406 in the passivation layer 403. The bond pad openings 406 provide openings for wire-bonding of bond wires to the bond pads 620 of the analysis chip.

As depicted in FIG. 21, a cap foundation layer 407 is preferably deposited on the base wafer 400. In the preferred embodiment of the invention, the cap foundation layer 407 comprises a deep ultraviolet (DUV) photopatternable thermoplastic layer, preferably a 3 μm thick PMMA layer. The cap foundation layer 407 is then patterned to create a foundation for a chip cap 417 that will be described later in this patent application.

The optional last step of the first stage of the second phase of the microfabrication process is depicted in FIG. 22 and will be described next. The fabrication process, for example, for the analysis chip does not incorporate the optional last step of the first stage of the second phase of the microfabrication process; however, the fabrication process, for example, for the analysis chip 3510 incorporates the optional last step of the first stage of the second phase of the microfabrication process. The optional last step of the first stage of the second phase of the microfabrication process commences with a hydrogel layer 408 being deposited on the base wafer 400. In a preferred embodiment of the invention, the hydrogel layer 408 comprises a 40 μm thick photopatternable hydrogel layer. The hydrogel layer 408 is then patterned to create one or more hydrogel features; the patterned hydrogel layer 408 provides one or more hydrogel valve actuators 452.

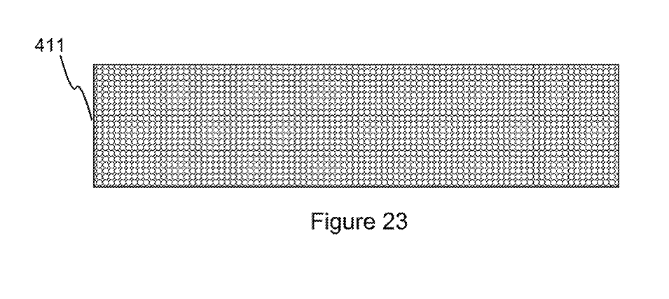

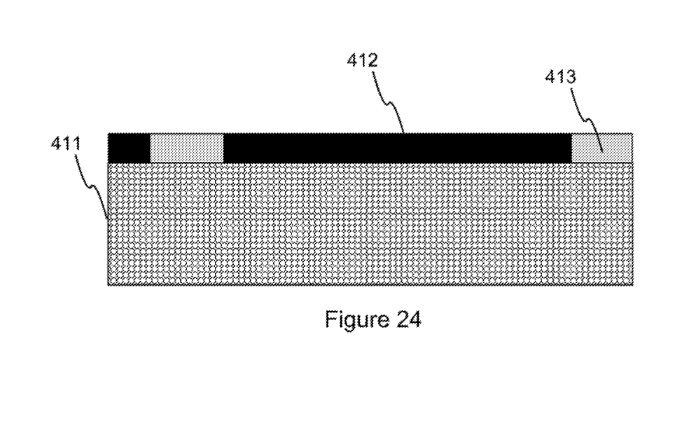

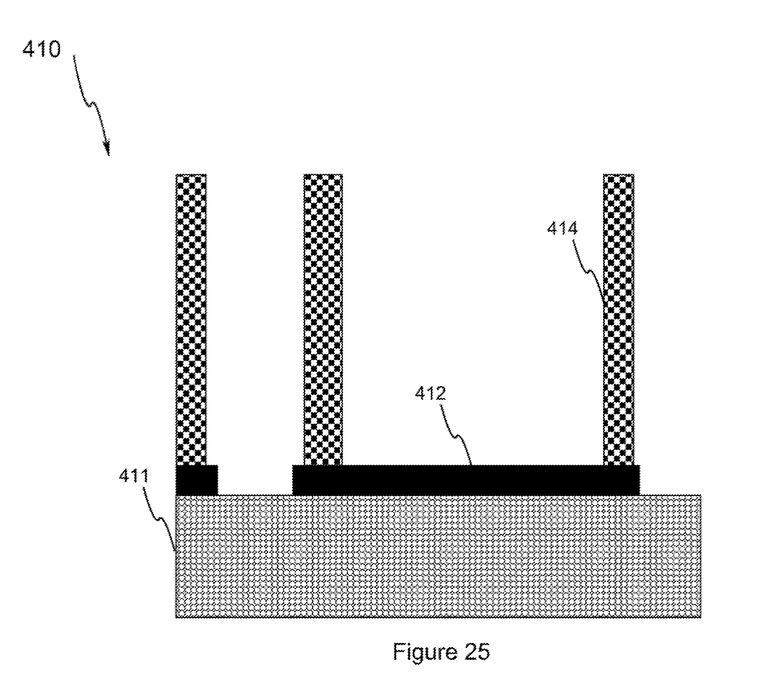

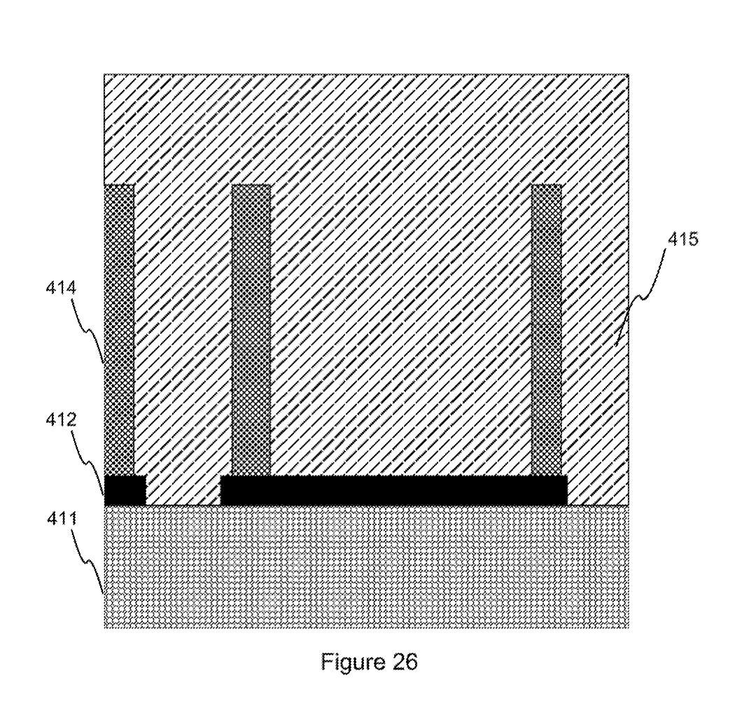

Referring to FIG. 23, FIG. 24, FIG. 25, FIG. 26, and FIG. 27, the second stage of the second phase of the microfabrication process for the microfabrication of the stamp 415 for the chip cap 417 will be described next.

As depicted in FIG. 23, the second stage of the second phase of the microfabrication process preferably begins with a mold substrate 411 for the fabrication of a mold 410. In the preferred embodiment of the invention, the mold substrate 411 comprises a silicon wafer or a quartz wafer.

As depicted in FIG. 24, a mold roof layer 412 is deposited on the mold substrate 411. In the preferred embodiment of the invention, the mold roof layer 412 comprises a negative photoresist, preferably a 5 μm thick layer of SU-8 photoresist or a functionally similar photoresist. The mold roof layer 412 is then exposed with the pattern of the regions that are to remain. The unexposed regions 413 are also shown in FIG. 24.

As depicted in FIG. 25, a mold wall layer 414 is deposited on the mold substrate 411. In the preferred embodiment of the invention, the mold wall layer 414 comprises a negative photoresist, preferably a 50 μm thick layer of SU-8 photoresist or a functionally similar photoresist. The mold wall layer 414 is then exposed. The mold roof layer 412 and the mold wall layer 414 are then together developed to complete the fabrication of the mold 410. In the way of emphasis, it should be noted that the unexposed regions 413, shown in FIG. 24, are “developed out” (i.e. removed) at this stage.

See FIG. 26 and FIG. 27. A stamp release layer (not shown) is optionally dispensed on the mold 410. An elastomer, preferably PDMS (poly(dimethylsiloxane)), is then dispensed on the mold 410 and subsequently cured to form the stamp 415. The stamp 415 is then released (as shown in FIG. 27), ready to be used in the fabrication of the chip cap 417.

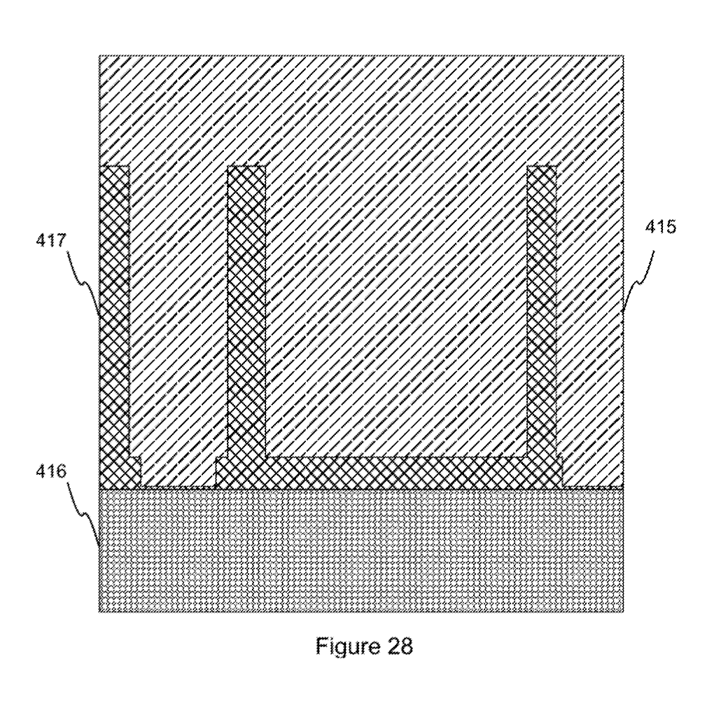

Referring to FIG. 28 and FIG. 29, the third stage of the second phase of the microfabrication process for the fabrication of the chip cap 417 using the stamp 415, which was previously fabricated, will be described next.

As depicted in FIG. 28, the third stage of the second phase of the microfabrication process preferably begins with a handle wafer 416. In the preferred embodiment of the invention, the handle wafer 416 comprises a quartz wafer. A polymer, preferably a thermoplastic, e.g., PMMA (poly(methyl methacrylate)), is then dispensed on the handle wafer 416 and subsequently hot-embossed with the stamp 415, followed by an oxygen reactive ion etch to remove the residual layer remaining after the hot-embossing step, to form the chip cap 417, as shown in FIG. 29.

Referring to FIG. 30, the fourth stage of the second phase of the microfabrication process is described now. The chip cap 417 is solvent-bonded to the base wafer 400, forming a stack 419 comprising the base wafer 400, the chip cap 417, and the handle wafer 416.

Referring to FIG. 31, the fifth stage of the second phase of the microfabrication process will be described next.

The stack 419 of the base wafer 400, the chip cap 417, and the handle wafer 416 is preferably mounted on a film frame and the handle wafer 416 is released (see FIG. 31). The analysis chips are singulated next, completing the second phase of the microfabrication process of the analysis chip.

As stated previously, the first transducer layer 405 is patterned to create structures, which include a plurality of heaters 680 and a plurality of electrodes 690. The first transducer layer 405 preferably comprises one of a metal, a ceramic, or a semiconductors, which more preferably is selected from a list consisting of platinum, platinum nanoparticles, gold, gold nanoparticles, Au-Pt alloy nanoparticles, silver, silver nanoparticles, silicon, silicon nitride, gallium nitride, ITO, FTO, TiO₂ on FTO, and In/Ga, which still more preferably is gold. In several of the embodiments, of the invention, the first transducer layer 405 comprises one of gold or platinum and is fabricated via one of the standard microfabrication techniques.

Metals may be deposited from the vapor phase or via solution processing. Au-Pt alloy nanoparticles may be deposited directly onto silicon (H-terminated) from a solution of HAuCl₄ and K₂PtCl₄ without further processing. (Zhao, Heinig, & Leung, 2012)

In some embodiments, to enhance immobilization of nucleic acid probes or other electrochemiluminescent components, first transducer layer 405 is chosen to incorporate suitable binding sites on the surface by fabricating it out of one of graphene, graphene oxide, carbon nanotubes (CNTs), pyrolytic graphite, glassy carbon, carbon coated with Nafion, conducting polymers such as PEDOT, PEDOT-PSS/Nafion, and PANI. In some embodiments, first transducer layer 405 comprising of TiO₂ provides an excellent substrate for catechol binding while CNTs and graphene bind to phosphate-terminated probes. CNTs adsorbed with streptavidin provide a good substrate for biotin-functionalized probes (Martinez, Tseng, Gonzalez, & Bokor, 2012). In some embodiments, first transducer layer 405 comprises gold, which binds strongly to thiol-terminated probes.

In some embodiments, first transducer layer 405 comprises high quality graphene, which is deposited, via chemical vapor deposition, directly on the top silicon nitride surface of the passivation layer 403 to give an electrode surface with high carrier mobility (Chen, et al., 2013). Graphene is a desirable surface due to its high affinity for oligonucleotides.

A second Embodiment of Assay Device

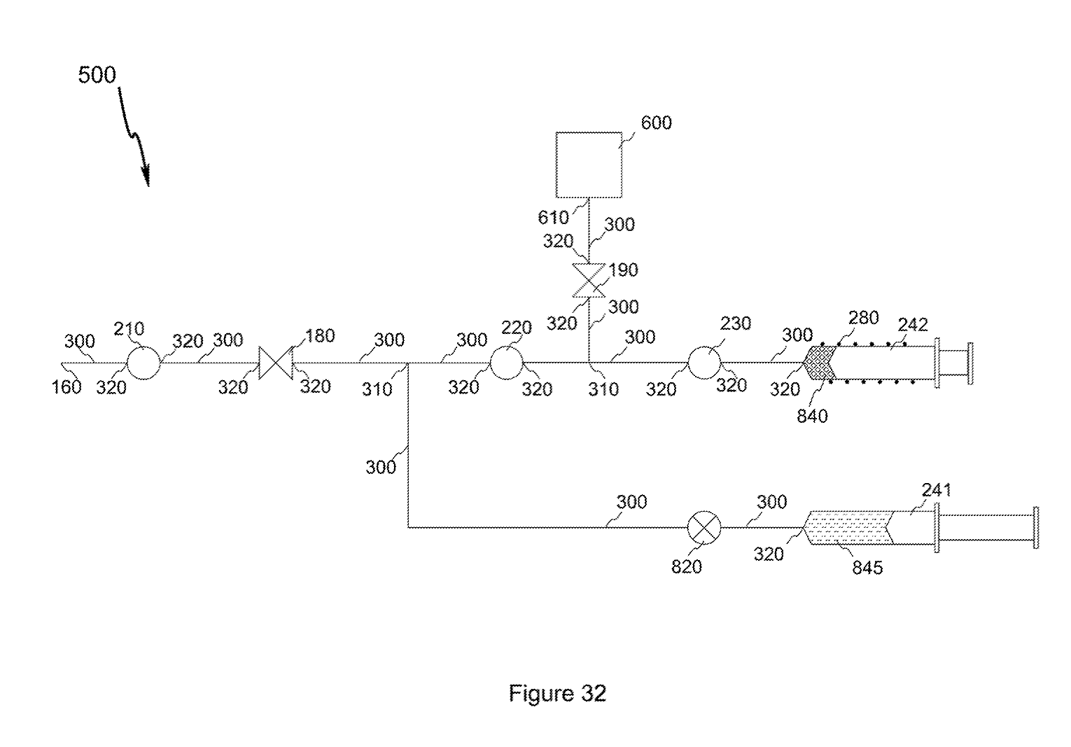

An assay device 500 will be described next. The assay device 500 is a second embodiment of the assay device, and it incorporates subsystems that are depicted schematically in FIG. 32.

The subsystems of some of the embodiments of the assay device 500 are divided into an expendable part or block 120 (as shown by reference number 120 in FIG. 1 and FIG. 2 for the analogous configuration relating to the assay device 100) and a reusable part or block 130 (as shown by reference number 130 in FIG. 1 and FIG. 2 for the analogous configuration relating to the assay device 100). The reusable part/block 130 is releasably engageable with the disposable part/block 120. Referring to these embodiments, the components of the assay device 500 that are preferably incorporated in the expendable part/block include a capillary tube inlet 160, preferably a breakable fluidic seal 820, a first valve 180, a second valve 190, a first liquid sensor 210, a second liquid sensor 220, a third liquid sensor 230, a first syringe pump 241, a second syringe pump 242, a syringe pump heating sleeve 280, an analysis module 600, a vent (not shown), and a plurality of fluidic interconnections 300 that interconnect these components. Each of the first syringe pump 241 and the second syringe pump 242 in turn incorporates a syringe barrel 250 and a piston 260.

Some embodiments of the assay device 500 incorporates an analysis module 3500 in place of the analysis module 600.

The second syringe pump 242 comprises a syringe barrel 250 and a piston 260. The syringe pump heating sleeve 280 is for heating the contents of the syringe barrel 250 as required.

While the syringe barrels 250 and the pistons 260 are part of the expendable part/block, a linear actuator 270 that drives the first syringe pump 241 and a linear actuator 270 that drives the second syringe pump 242 are part of the reusable part/block.

When the expendable block is inserted into the reusable block and pressed in, a mechanical connection is formed between the linear actuator 270 that drives the first syringe pump 241 and the piston 260 of the first syringe pump 241, and a mechanical connection is formed between the linear actuator 270 that drives the second syringe pump 242 and the piston 260 of the second syringe pump 242, allowing for each of the actuators to pull each of the pistons 260 out by retracting the linear actuator 270 connected to the said piston 260 or push each of the pistons 260 in by extending the linear actuator 270 connected to the said piston 260, as required, to respectively draw a fluid into each of the syringe barrels 250 or force the fluid out of each of the syringe barrels 250.

The fluidic interconnections 300 of the assay device 500 preferably comprise tubing with an internal diameter between 10 μm and 4 mm, more preferably between 20 μm and 2 mm, still more preferably between 50 μm and 1 mm, still more preferably between 50 μm and 500 μm, and most preferably between 50 μm and 300 μm. In one embodiment, the internal diameter of the tubing is between 100 μm and 200 μm.

Still referring to FIG. 32, the capillary tube inlet 160 is connected, via the fluidic interconnection 300, to a first fluidic port 320 of the first liquid sensor 210. It should be noted that, in the preferred embodiment of the invention, the capillary tube inlet 160 is simply the open end of a capillary tube that constitutes the fluidic interconnection 300 that is connected to the first fluidic port 320 of the first liquid sensor 210.

A second fluidic port 320 of the first liquid sensor 210 is connected to a first fluidic port 320 of the first valve 180 via the fluidic interconnection 300.

A second fluidic port 320 of the first valve 180 is connected to a first fluidic intersection 310 via the fluidic interconnection 300. The first fluidic intersection 310 is connected, via the fluidic interconnections 300, to a first fluidic port 320 of the second liquid sensor 220 and, past the breakable fluidic seal 820, to a fluidic port 320 of the first syringe pump 241.

A second fluidic port 320 of the second liquid sensor 220 is connected to a second fluidic intersection 310 via the fluidic interconnection 300. The second fluidic intersection 310 is additionally connected, via the fluidic interconnections 300, to a first fluidic port 320 of the second valve 190 and to a fluidic port 320 of the third liquid sensor 230.

A second fluidic port 320 of the second valve 190 is connected to the analysis module inlet 610 of the analysis module 600 via the fluidic interconnection 300.

A second fluidic port 320 of the third liquid sensor 230 is connected to a fluidic port 320 of the second syringe pump 242 via the fluidic interconnection 300.

The second syringe pump 242 is fitted with a heating sleeve 280.

In some embodiments, the content of the syringe barrel of the first syringe pump 241 comprises one or more liquid reagents 845. The one or more liquid reagents 845 can also simply be “water” or a buffer.

In some embodiments, the content of the syringe barrel of the second syringe pump 242 comprises one or more assay reagents 840. The assay reagents are loaded in the syringe barrel of the second syringe pump 242 during the assembly of the assay device expendable block. In the preferred embodiments of the invention, the assay reagents are stored in the syringe barrel of the second syringe pump 242 in a dry form.

In the embodiments of the assay device 500 that incorporate the breakable fluidic seal 820, the breakable fluidic seal 820 isolates the content of the first syringe pump 241 before use. During use, upon the initiation of the forcing of the fluid out of the syringe barrels 250 of the first syringe pump 241, the breakable fluidic seal 820 will break and allow the fluid to flow in the direction of the first fluidic intersection 310.

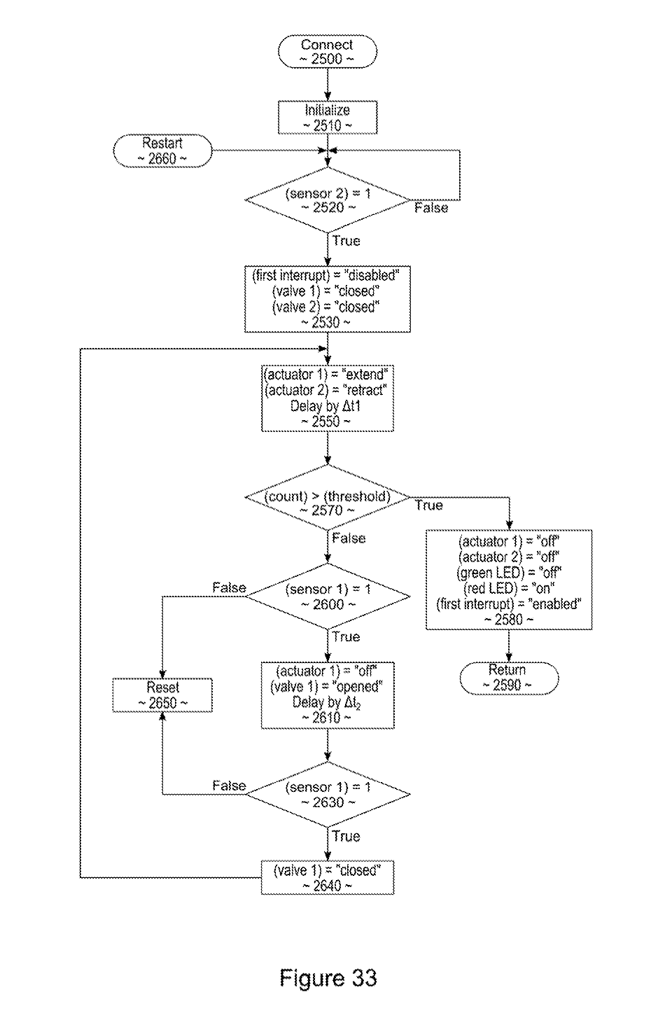

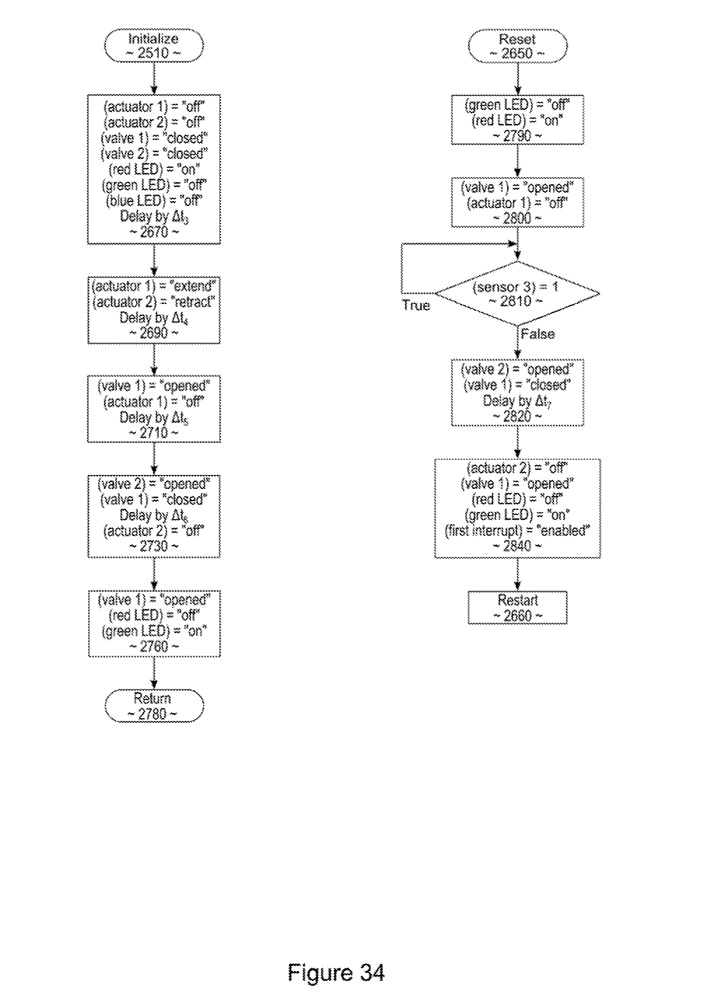

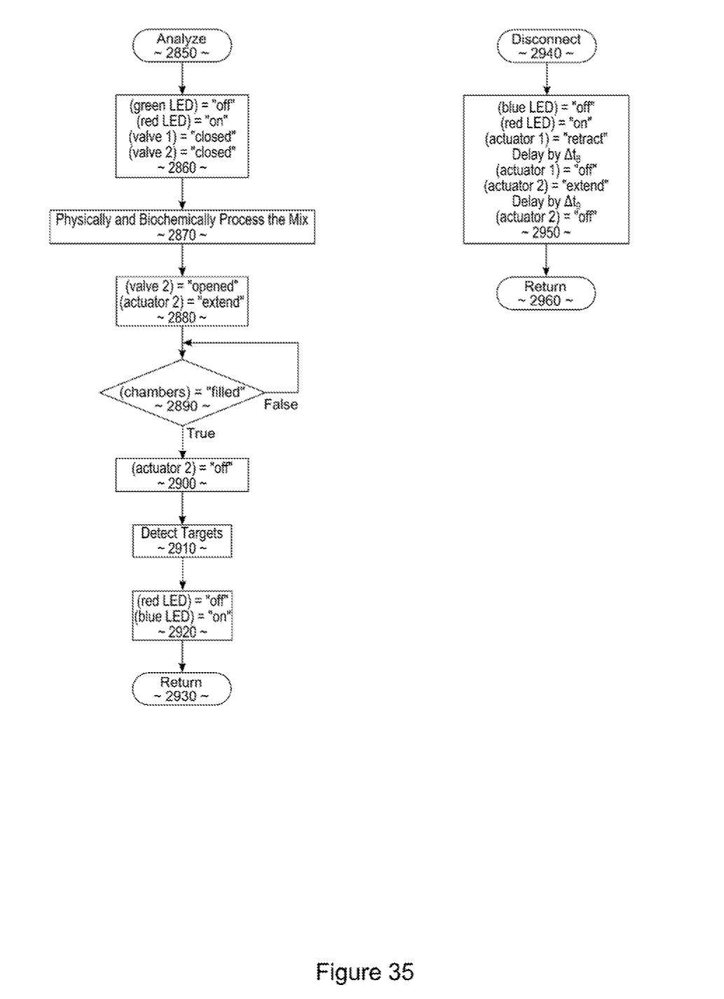

A flow diagram describing one of the many variants of the operation of the assay device 500 is shown in FIG. 33, FIG. 34, and FIG. 35. It will be appreciated that this flow diagram only describes the more significant aspects of only one variant of the operation of the assay device 500. As described in the following, the operation of the assay device 500 is highly automated, and only a limited degree of user input is required in collecting and analyzing the sample.

After the connection between the assay device 500 and the host is made, the control is transferred to process 2500 through an interrupt.

Following the process 2500, the assay device is initialized (process 2510). The initialization (process 2510) commences with the process 2670 (see FIG. 34) where the linear actuator driving the first syringe pump 241 is set to the “off” state, the linear actuator driving the second syringe pump 242 is set to the “off” state, the first valve 180 is set to the “closed” state, and the second valve 190 is set to the “closed” state (preventing the fluid to pass through the valves). After this, the red LED 142 on the assay device's reusable block is turned on, and the green LED 144 and the blue LED 146 on the assay device's reusable block are turned off, signaling to the user to wait for the initialization to complete. This is followed by a suitable time delay of Δt₃ a to allow for the assay device 500 to settle to the final states that are mandated by the instructions of the process 2670.

It should be noted that in the preferred embodiment of the invention, the assay device 500 is designed in such a way that, upon the “power-up” of the assay device 500, certain of the “signal lines” of the assay device 100 are “pulled up” or “pulled down”, as required, to put the assay device 500 in the final states that are mandated by the instructions of the process 2670, in a “glitch-free” manner, even before the signals are asserted by the controller as part of the process 2670.

After process 2670, control is transferred to process 2690. Process 2690 comprises of setting the linear actuator that drives the piston of the first syringe pump 241 to extend, setting the linear actuator that drives the piston of the second syringe pump 242 to retract, and delaying for a suitable period of time Δt₄ to assure that the one or more liquid reagents 845 that are forced out of the first syringe pump 241 go past the first fluidic intersection 310 and some ways toward the second syringe pump 242.

In the embodiment just described, the process 2690 is a “timed” process, where the time delay Δt₄ is predetermined in such a way for a suitable volume of the one or more liquid reagents 845 to be forced out of the first syringe pump 241 and go past the first fluidic intersection 310 and some ways toward the second syringe pump 242. In other embodiments, the process 2690 is not a timed process, and instead other techniques are used in assuring the suitable volume of the one or more liquid reagents 845 is forced out of the first syringe pump and into the flow path between the first fluidic intersection 310 and the second syringe pump 242. In one embodiment, the technique that is used to assure the suitable volume of the one or more liquid reagents 845 is forced out of the first syringe pump and into the flow path between the first fluidic intersection 310 and the second syringe pump 242 comprises a “positional sensor” that is “linked” to the linear actuator that drives the piston of the first syringe pump 241. The output of the positional sensor is used to provide feedback to suitably control the volume of the one or more liquid reagents 845 that is forced out of the first syringe pump and into the flow path between the first fluidic intersection 310 and the second syringe pump 242. In another embodiment, the technique that is used to assure the suitable volume of the one or more liquid reagents 845 is forced out of the first syringe pump and into the flow path between the first fluidic intersection 310 and the second syringe pump 242 comprises the second liquid sensor 220 or the third liquid sensor 230; in this embodiment, the first syringe pump 241 is turned off when the second liquid sensor 220 or the third liquid sensor 230 indicates the presence of liquid in the flow path between the first fluidic intersection 310 and the second syringe pump 242. This technique works to suitably control the volume of the one or more liquid reagents 845 that is forced out of the first syringe pump and into the flow path between the first fluidic intersection 310 and the second syringe pump 242.

After the completion of the process 2690, control is transferred to process 2710. Process 2710 comprises of the first valve 180 being set to the “open” state (allowing the fluid to pass through the valve), the linear actuator driving the first syringe pump 241 being set to the “off” state, and delaying for a suitable period of time Δt₅. This draws the liquid in the flow path between the first fluidic intersection 310 and the second syringe pump 242 towards the second syringe pump 242, without drawing any new liquid out of the first syringe pump 241. The process 2710 assures that there is no liquid in the flow path between the capillary tube inlet of the assay device 500 and the first fluidic port 320 of the liquid sensor 230.

After the completion of the process 2710, control is transferred to process 2730. Process 2730 comprises of opening the second valve 190, closing the first valve 180, delaying for a suitable period of time Δt₆, and then setting the linear actuator driving the second syringe pump 242 to the “off” state. This draws any liquid possibly existing in the flow path between the first fluidic port 320 of the second valve 190 and the first fluidic port 320 of the liquid sensor 230 towards the second syringe pump 242, again, without drawing any new liquid out of the first syringe pump 241.

In the embodiment just described, the process 2730 is a “timed” process, where the time delay Δt₆ is predetermined in such a way as to draw any liquid possibly existing in the flow path between the first fluidic port 320 of the second valve 190 and the first fluidic port 320 of the liquid sensor 230 towards the second syringe pump 242. In other embodiments, the process 2730 is not a timed process, and instead other techniques are used to assure to draw any liquid possibly existing in the flow path between the first fluidic port 320 of the second valve 190 and the first fluidic port 320 of the liquid sensor 230 towards the second syringe pump 242. In one embodiment, the technique that is used to assure to draw any liquid possibly existing in the flow path between the first fluidic port 320 of the second valve 190 and the first fluidic port 320 of the liquid sensor 230 towards the second syringe pump 242 comprises a “positional sensor” that is “linked” to the linear actuator that drives the piston of the second syringe pump 242. The output of the positional sensor is used to provide feedback to suitably control the drawing of any liquid possibly existing in the flow path between the first fluidic port 320 of the second valve 190 and the first fluidic port 320 of the liquid sensor 230 towards the second syringe pump 242. In another embodiment, the technique that is used to assure to draw any liquid possibly existing in the flow path between the first fluidic port 320 of the second valve 190 and the first fluidic port 320 of the liquid sensor 230 towards the second syringe pump 242 comprises the third liquid sensor 230; in this embodiment, the second syringe pump 242 is turned off when the third liquid sensor 230 indicates the drawing of any liquid possibly existing in the flow path between the first fluidic port 320 of the second valve 190 and the first fluidic port 320 of the liquid sensor 230 towards the second syringe pump 242. This technique works to suitably control the drawing of any liquid possibly existing in the flow path between the first fluidic port 320 of the second valve 190 and the first fluidic port 320 of the liquid sensor 230 towards the second syringe pump 242.

After the completion of the process 2730, control is transferred to process 2760. Process 2760 comprises of opening the first valve 180 thus making the assay device 500 ready for the collection of the sample. After this, the red LED 142 on the reusable block of the assay device is turned off and the green LED 144 on the reusable block is turned on, signaling to the user that the procedure for the collection of the sample can commence.

After the completion of the process 2760, process 2780 simply returns the control back to the process 2520 (see FIG. 33).

At this stage, the one or more liquid reagents 845 form a meniscus at the first fluidic intersection 310, with the fluidic path between the capillary tube inlet 160 and the first fluidic port 320 of the third liquid sensor 230 being empty of any liquid.

For the NAF sample, the NAF is aspirated according to one of the standard NAF aspiration procedures.

To collect the sample, the user brings the capillary tube inlet 160 of the assay device 500 into contact with the aspirated NAF (or the liquid sample of a different type). The liquid sample is drawn, via capillary action, sequentially through the capillary tube inlet 160, the first liquid sensor 210, the first valve 180, the first fluidic intersection 310, and the second liquid sensor 220.

When the sample reaches the second liquid sensor 220, the presence of the sample is detected by this sensor. This is depicted as process 2520 in the flow diagram.

The detection of the sample by the second liquid sensor 220 is followed by process 2530 where a “first interrupt” is disabled (the reason for this instruction will be clarified in the subsequent processes), and the first valve 180 and the second valve 190 are closed (thereby closing the fluidic path and stopping the flow of the liquid sample).

At this stage, as part of process 2550, the linear actuator 270 driving the piston 260 of the first syringe pump 241 is set to extend and the linear actuator 270 driving the piston 260 of the second syringe pump 242 is set to retract for a time period determined by a time delay Δt₁. This action propels the slug of the sample that occupies the fluidic path between the first fluidic intersection 310 and the second liquid sensor 220 towards the second syringe pump 242, with the one or more liquid reagents 845, which are being forced out of the first syringe pump 241, following the slug and partially mixing with it.

Process 2570 follows the process 2550 and monitors a “count”, which is a number representing the volume of the sample that has been collected by the assay device. If the count exceeds a “threshold”, which corresponds to the maximum volume of the sample that the design is capable of collecting, process 2580 will be executed. The process 2580 is one of the flow paths that will eventually lead into the analysis phase of the assay device operation and will be described later in this specification. If the count is less than the threshold, process 2600 will be executed, which will allow for more of the sample to be collected. The process 2600 will also be described later in this specification.

The process 2580 turns the linear actuator 270 driving the piston 260 of the first syringe pump 241 off, turns the linear actuator 270 driving the piston 260 of the second syringe pump 242 off, turns the green LED 144 off, and turns the red LED 142 on, indicating to the user that no more sample is to be collected. The process then enables the first interrupt, allowing an “interrupt-driven” analysis phase of the sample to be commenced. The diagnostic, prognostic, and analytical system then, as indicated in process 2590 in the flow diagram, returns the control to higher-level host software and remains ready for the analysis phase (commencing with process 2850, FIG. 35).

The process 2600, however, checks the output of the first liquid sensor 210. If the first liquid sensor 210 indicates the detection of more of the sample in the interior of the sensor, process 2610 will be executed. The process 2610 will be described later in this specification. If the first liquid sensor 210 detects a depletion of the liquid sample in the interior of the sensor, process 2650 will be executed to reset the sample collection processes and allow for more of the sample to be collected if desired. The process 2650 will be described later in this specification. This condition, where the first liquid sensor 210 detects a depletion of the liquid sample in the interior of the sensor, can be due to a situation where the supply of the sample has been exhausted or the contact between the capillary tube inlet 160 and the aspirated NAF sample has intentionally or unintentionally been lost.

As part of process 2610, the linear actuator 270 driving the piston 260 of the first syringe pump 241 is turned off and the first valve 180 is opened for a time period determined by a time delay Δt₂. With the linear actuator driving the second syringe pump 242 still being retracted, this action propels more of the sample that occupies the fluidic path between the first liquid sensor 210 and the first fluidic intersection 310 towards the second syringe pump 242.

Then process 2630 again checks the output of the first liquid sensor 210. If the first liquid sensor 210 indicates the detection of more of the sample in the interior of the sensor, process 2640 will be executed. As part of the process 2640, the first valve 180 is closed. Then the control is transferred to the process 2550 for a new sample collection cycle to commence. If, as part of the process 2630, the first liquid sensor 210 detects a depletion of the liquid sample in the interior of the sensor, the process 2650 will be executed to reset the sample collection processes and allow for more of the sample to be collected if desired. The process 2650 will be described later in this specification. This condition, where the first liquid sensor 210 detects a depletion of the liquid sample in the interior of the sensor, can be due to a situation where the supply of the sample has been exhausted or the contact between the capillary tube inlet 160 and the aspirated NAF sample has intentionally or unintentionally been lost.

As mentioned previously, the process 2650 is to reset the sample collection processes and allow for more of the sample to be collected if desired. The process 2650 transfers the control to process 2790.

Process 2790 turns the green LED 144 off and turns the red LED 142 on, indicating to the user that no more sample is to be collected until when the red LED 142 is turned off again and the green LED 144 is on again. The process then transfers the control to process 2800.

Process 2800 opens the first valve 180, turns the linear actuator 270 driving the piston 260 of the first syringe pump 241 off, and transfers the control to process 2810. With the linear actuator driving the second syringe pump 242 still being retracted, this action propels more of the sample that occupies the fluidic path between the first liquid sensor 210 and the third liquid sensor 230 towards the second syringe pump 242.

The process 2810 checks the output of the third liquid sensor 230. For the duration that the third liquid sensor 230 indicates the detection of the sample in the interior of the sensor, process 2810 will be repeated. When the third liquid sensor 230 detects a depletion of the liquid sample in the interior of the sensor, process 2820 will be executed. The condition, where the third liquid sensor 230 detects a depletion of the liquid sample in the interior of the sensor, indicates that the fluidic path from the capillary tube intake to the third liquid sensor 230 is depleted of the sample and the one or more liquid reagents.

The process 2820 opens the second valve 190 and closes the first valve 180 for a time period determined by a time delay Δt₇. With the linear actuator driving the second syringe pump 242 still being retracted, this action draws any liquid possibly existing in the flow path between the first fluidic port 320 of the second valve 190 and the first fluidic port 320 of the liquid sensor 230 towards the second syringe pump 242.

In the embodiment just described, the process 2820 is a “timed” process, where the time delay Δt₇ is predetermined in such a way as to draw any liquid possibly existing in the flow path between the first fluidic port 320 of the second valve 190 and the first fluidic port 320 of the liquid sensor 230 towards the second syringe pump 242. In other embodiments, the process 2820 is not a timed process, and instead other techniques are used in assuring to draw any liquid possibly existing in the flow path between the first fluidic port 320 of the second valve 190 and the first fluidic port 320 of the liquid sensor 230 towards the second syringe pump 242. In one embodiment, the technique that is used to assure to draw any liquid possibly existing in the flow path between the first fluidic port 320 of the second valve 190 and the first fluidic port 320 of the liquid sensor 230 towards the second syringe pump 242 comprises a “positional sensor” that is “linked” to the linear actuator that drives the piston of the second syringe pump 242. The output of the positional sensor is used to provide feedback to suitably control the drawing of any liquid possibly existing in the flow path between the first fluidic port 320 of the second valve 190 and the first fluidic port 320 of the liquid sensor 230 towards the second syringe pump 242. In another embodiment, the technique that is used to assure to draw any liquid possibly existing in the flow path between the first fluidic port 320 of the second valve 190 and the first fluidic port 320 of the liquid sensor 230 towards the second syringe pump 242 comprises the third liquid sensor 230; in this embodiment, the second syringe pump 242 is turned off when the third liquid sensor 230 indicates the drawing of any liquid possibly existing in the flow path between the first fluidic port 320 of the second valve 190 and the first fluidic port 320 of the liquid sensor 230 towards the second syringe pump 242. This technique works to suitably control the drawing of any liquid possibly existing in the flow path between the first fluidic port 320 of the second valve 190 and the first fluidic port 320 of the liquid sensor 230 towards the second syringe pump 242.

Process 2820 transfers the control to process 2840. The process 2840 turns the linear actuator driving the second syringe pump 242 off, opens the first valve 180, turns the red LED 142 off, turns the green LED 144 on, enables the first interrupt, and transfers control to process 2660 to restart any additional collection of the sample that may be desired. To affect the collection of any additional sample that may be desired, the process 2660 transfers the control to the process 2520. However, as the process 2840 has enabled the first interrupt, an interrupt-driven analysis phase of the sample can be commenced as desired.

Referring back to the process 2530, after enabling the first interrupt during the process 2840, the window for commencing the interrupt-driven analysis phase of the sample will last until the disabling of the first interrupt during the execution of the process 2530. However, when it is desired to commence the interrupt-driven analysis phase of the sample, naturally no more of the sample would be collected and the control would stay within the process 2520 and will not reach the instruction in the process 2530 that disables the first interrupt.

After the desired amount of the liquid sample has been collected, the analysis phase can commence. The analysis phase of the assay device operation begins by the user giving the appropriate “analyze” command to the assay device. This command can be given by momentarily depressing and releasing the tactile switch 150 on the assay device or via a command being initiated via the user interface of the assay device application software on the host. The analyze command is interrupt-driven, and it interrupts the control flow, transferring control to process 2850 in the flow diagram.

The analysis of the sample commences according to process 2860 in the flow diagram where the green LED 144 is turned off, indicating to the user that no more of the sample is to be collected, the red LED 142 is turned on, as the indication that the analysis phase is in progress, and the first valve 180 and the second valve 190 are closed.

The syringe barrel 250 of the second syringe pump 242 may optionally also store one or more assay reagents. The assay reagents are loaded in the syringe barrel 250 of the second syringe pump 242 during the assembly of the assay device expendable block. In the preferred embodiments of the invention, the assay reagents are stored in the syringe barrel 250 of the second syringe pump 242 in a dry form.

At this stage, the liquid sample that had been drawn into the syringe barrel 250 of the second syringe pump 242 has undergone some mixing with the assay reagents that had been stored in the syringe barrel 250. The resulting mix will be referred to as a chemical mix or a biochemical mix for the purpose of this patent application, and the resulting fluid will be referred to as the chemical mix or the biochemical mix irrespective of whether the assay reagents were stored in the syringe barrel 250 of the second syringe pump 242 or not and also irrespective of any subsequent processing that the chemical mix or the biochemical mix may still undergo.

The biochemical mix now undergoes a number of physical and biochemical processes (depicted as process 2870 in the flow diagram). These processes take place by appropriately heating the biochemical mix inside the syringe barrel 250 of the second syringe pump 242 via the heating sleeve 280 that surrounds the syringe barrel 250 of the second syringe pump 242. In effect, the syringe barrel 250 of the second syringe pump 242 additionally functions as a reaction chamber.

The physical and biochemical processes that take place in the reaction chamber (syringe barrel 250 of the second syringe pump 242, in this embodiment) include the mixing of the liquid sample with the assay reagents (also further mixing with the one or more liquid reagents), the lysis of any cells in the biochemical mix to release their genetic material, and the amplification of various desired segments of this genetic material. The genetic material includes one or more of DNA molecules, RNA molecules, and their methylated forms. The biochemical mix wherein the genetic material in the biochemical mix has undergone amplification is called the amplicon.

The physical and biochemical processing of the biochemical mix is a timed process. After the amplification of the genetic material in the biochemical mix has been completed, the second valve 190 is opened and the linear actuator 270 driving the second syringe pump 242 is set to force the biochemical mix out of the syringe barrel 250 and, through the analysis module inlet 610, into the analysis module 600 (depicted as process 2880 in the flow diagram). Enough of the biochemical mix is forced into the analysis module 600 to fill the detection chambers 650 that are incorporated in the analysis module 600. The method by which the filling of the detection chambers 650 of the analysis module 600 is determined relies on one or more of the liquid sensors 200 that are located in the flow path in the analysis module 600 in such a way as to be able to ascertain the filled state of the detection chambers 650 in the analysis module 600. After a suitable time interval after the liquid sensors 200 detect the presence of liquid in the “sensors' manifolds”, it can be assumed that all of the detection chambers 650 are filled; therefore, the second syringe pump 242 continues to force the biochemical mix into the detection chambers 650 of the analysis module 600 until after the liquid sensors 200 have indicated the presence of the liquid and after the suitable time interval after the detection of the liquid has elapsed (depicted as process 2890 in the flow diagram).

At this stage, as depicted as process 2900 in the flow diagram, the linear actuator 270 driving the second syringe pump 242 is turned off and the detection of the various target molecules (target nucleic acid sequences or various target proteins), contained in the biochemical mix, can now commence (as depicted in process 2910 in the flow diagram).

Each of the detection chambers 650 in the analysis module 600 includes a probe specific to a particular analyte. The analyte may be a nucleic acid sequence. Each of the detection chambers 650 includes an electrochemiluminescence resonance energy transfer probe that is anchored to an interior surface of the detection chamber 650. For the purpose of this patent application, the chemical mix or the biochemical mix forced into the detection chambers 650 and the electrochemiluminescence resonance energy transfer probe will together be referred to as the contents of the detection chambers 650. The analysis module 600 comprises a plurality of heaters 680 for heating the contents of the detection chambers 650. When a nucleic acid sequence in the biochemical mix is complementary to the electrochemiluminescence resonance energy transfer probe in a detection chamber 650, the nucleic acid sequence hybridizes with the electrochemiluminescence resonance energy transfer probe and forms a hybrid, indicating the presence of a target molecule (a target nucleic acid sequence).

Electrical excitation of the content of the detection chambers 650 in parallel to the detection of any emission by the electrochemiluminescence resonance energy transfer probes in the detection chambers is used to detect the existence of hybrids in each of the detection chambers 650.

Each of the detection chambers 650 incorporate two excitation electrodes 690, an anode and a cathode. Additionally, the analysis chip comprises a plurality of photosensors 710 adjacent to the detection chambers 650 for detecting any emission by the electrochemiluminescence resonance energy transfer probes in the detection chambers 650. An excitation current is forced between the two electrodes 690 in each of the detection chambers 650, and any electrochemiluminescence emission by the electrochemiluminescence resonance energy transfer probe is detected by the photosensors 710 adjacent to the detection chambers 650. Under this arrangement, the detection of the emission by the electrochemiluminescence resonance energy transfer probes in the detection chambers 650 correspond to the detection of the nucleic acid sequences complementary to the probes that have been loaded in the detection chambers.

The hybridization data gathered, in the manner described, is further processed by the host and compared to the database of the diagnostic markers (e.g., disease markers) and/or prognostic markers to meet the diagnostic and prognostic objectives.

At this stage, with the detection of the presence of the diagnostic markers and/or prognostic markers having been completed, the host turns the red LED 142 off and the blue LED 146 on, indicating to the user that the analysis results are ready. This is depicted as process 2920 in the flow diagram. In parallel to the indication given to the user by the blue LED 146, the user interface of the application software running on the host also provides an indication to the user of the completion of the detection and analysis process.

At this stage, the user can use the user interface of the application software on the host to view or further process the results of the analysis of the sample.

After the completion of the process 2920, process 2930 returns the control to higher-level host software.

At this stage, the user may remove the expendable block of the assay device 500 and dispose of it accordingly. Removing the expendable block triggers an interrupt, which transfers the control to process 2940, which in turn transfers control to process 2950.

As indicated in the flow diagram, during the execution of the process 2950, the blue LED 146 is turned off, the red LED 142 is turned on, the linear actuator 270 that drives the first syringe pump 241 is set to retract, and after a time delay of Δt₈, the linear actuator 270 that drives the first syringe pump 241 is set to the off state, and the linear actuator 270 that drives the second syringe pump 242 is set to extend, and after a time delay of Δt₉, the linear actuator 270 that drives the second syringe pump 242 is set to the off state. The time delay Δt₈ and the time delay Δt₉ are determined by the application software in such a way that the linear actuator 270 that drives the first syringe pump 241 is suitably retracted and the linear actuator 270 that drives the second syringe pump 242 is suitably extended to make the reusable block of the assay device 500 ready for receiving an unused unit of the expendable block for another analysis cycle, if desired. Then, as indicated in process 2960, the control is returned to higher-level application software, and the diagnostic, prognostic, and analytical system waits for any desired further use.

In the embodiment just described, the process 2950 is a “timed” process, where the time delay Δt₈ and the time delay Δt₉ are determined by the application software in such a way that the linear actuator 270 that drives the first syringe pump 241 is suitably retracted and the linear actuator 270 that drives the second syringe pump 242 is suitably extended to make the reusable block of the assay device 500 ready for receiving an unused unit of the expendable block. In other embodiments, the process 2950 is not a timed process, and instead other techniques are used in assuring that the linear actuator 270 that drives the first syringe pump 241 is suitably retracted and the linear actuator 270 that drives the second syringe pump 242 is suitably extended to make the reusable block of the assay device 500 ready for receiving an unused unit of the expendable block. In one embodiment, the technique that is used to assure that the linear actuator 270 that drives the first syringe pump 241 is suitably retracted and the linear actuator 270 that drives the second syringe pump 242 is suitably extended to make the reusable block of the assay device 500 ready for receiving an unused unit of the expendable block comprises a first “positional sensor” that is “linked” to the linear actuator 270 that drives the first syringe pump 241 and a second “positional sensor” that is “linked” to the linear actuator 270 that drives the second syringe pump 242. The outputs of the first positional sensor and the second positional sensor are used to provide feedback to suitably control the linear actuator 270 that drives the first syringe pump 241 is suitably retracted and the linear actuator 270 that drives the second syringe pump 242 is suitably extended to make the reusable block of the assay device 500 ready for receiving an unused unit of the expendable block.

### A Third Embodiment of Assay Device

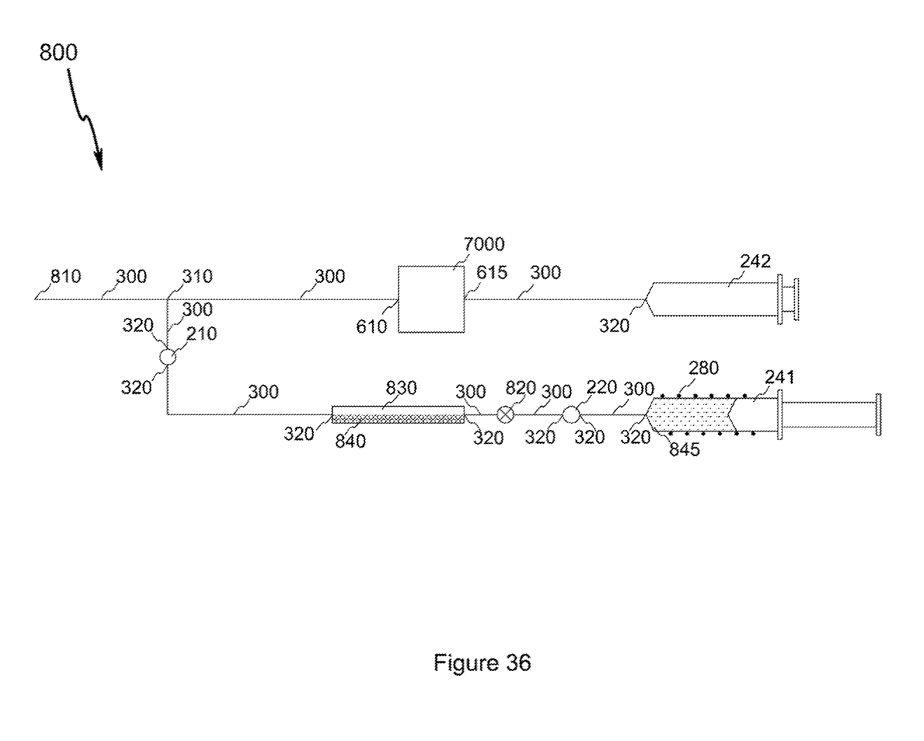

An assay device 800 will be described next. The assay device 800 is a third embodiment of the assay device, and it is depicted schematically in FIG. 36. The schematic comprises only the more important features of the assay device 800, and the auxiliary or optional features are left out for the sake of simplicity. The subsystems of the preferred embodiments of the assay device 800 are divided into an expendable part or block 120 (as shown by reference number 120 in FIG. 1 and FIG. 2 for the analogous configuration relating to the assay device 100) and a reusable part or block 130 (as shown by reference number 130 in FIG. 1 and FIG. 2 for the analogous configuration relating to the assay device 100). The reusable part/block 130 is releasably engageable with the disposable part/block 120. Referring to these embodiments, the components of the assay device 800 that are preferably incorporated in the expendable part/block include an inlet 810, a breakable fluidic seal 820, a first syringe pump 241, a second syringe pump 242, a reagent chamber 830, a heating sleeve 280 for the first syringe pump 241, an analysis module 7000, a first liquid sensor 210, a second liquid sensor 220, a vent (not shown), and a plurality of fluidic interconnections 300 that interconnect these components. Each of the first syringe pump 241 and the second syringe pump 242 in turn incorporates a syringe barrel 250 and a piston 260. The second syringe pump 242 is operable to draw fluid via suction through the analysis module 7000 (more details will be described later). It will be appreciated that various embodiments of assay device 800 can comprise other embodiments of the analysis module, including those described elsewhere in this specification.

The syringe pump heating sleeve 280 is for heating the contents of the syringe barrel 250 of the first syringe pump 241 as required.

While the syringe barrels 250 and the pistons 260 are part of the expendable part/block, a linear actuator 270 that drives the first syringe pump 241 and a linear actuator 270 that drives the second syringe pump 242 are part of the reusable part/block.

When the expendable block is inserted into the reusable block and pressed in, a mechanical connection is formed between the linear actuator 270 that derives the first syringe pump 241 and the piston 260 of the first syringe pump 241, and a mechanical connection is formed between the linear actuator 270 that drives the second syringe pump 242 and the piston 260 of the second syringe pump 242, allowing for each of the actuators to be able to pull each of the pistons 260 out by retracting the linear actuator 270 connected to the said piston 260 or push each of the pistons 260 in by extending the linear actuator 270 connected to the said piston 260, as required, to respectively draw a fluid into each of the syringe barrels 250 or force the fluid out of each of the syringe barrels 250.

The fluidic interconnections 300 of the assay device 800 preferably comprise tubing with an internal diameter between 10 μm and 4 mm, more preferably between 20 μm and 2 mm, still more preferably between 50 μm and 1 mm, still more preferably between 50 μm and 500 μm, and most preferably between 50 μm and 300 μm. In one embodiment, the internal diameter of the tubing is between 100 μm and 200 μm.

Still referring to FIG. 36, the inlet 810 is connected, via the fluidic interconnection 300, to a fluidic intersection 310. The fluidic intersection 310 is connected, via the fluidic interconnections 300, to an analysis module inlet 610 of the analysis module 7000 and a first fluidic port 320 of the first liquid sensor 210. It should be noted that, in the preferred embodiment of the invention, the inlet 810 is simply the open end of a tube that constitutes the fluidic interconnection 300 that is connected to the inlet 810.

A second fluidic port 320 of the first liquid sensor 210 is connected, via the fluidic interconnection 300, to a first fluidic port of a reagent chamber 830 that may optionally store one or more assay reagents 840. The one or more assay reagents 840 are loaded in the reagent chamber 830 during the assembly of the assay device expendable block. In the preferred embodiments of the invention, the one or more assay reagents 840 are stored in the reagent chamber 830 in a dry form.

A fluidic port 320 of the first syringe pump 241 is connected, via the fluidic interconnections 300, to a first fluidic port 320 of the second liquid sensor 220. The second fluidic port 320 of the second liquid sensor 220 is connected, via the fluidic interconnections 300 and the breakable fluidic seal 820, to a second fluidic port 320 of the reagent chamber 830.

In the embodiments of the assay device 800 that incorporate the breakable fluidic seal 820, the breakable fluidic seal 820 isolates the content of the first syringe pump 241 before use. During use, upon the initiation of the forcing of the fluid out of the syringe barrels 250 of the first syringe pump 241, the breakable fluidic seal 820 will break and allow the fluid to enter the reagent chamber 830.

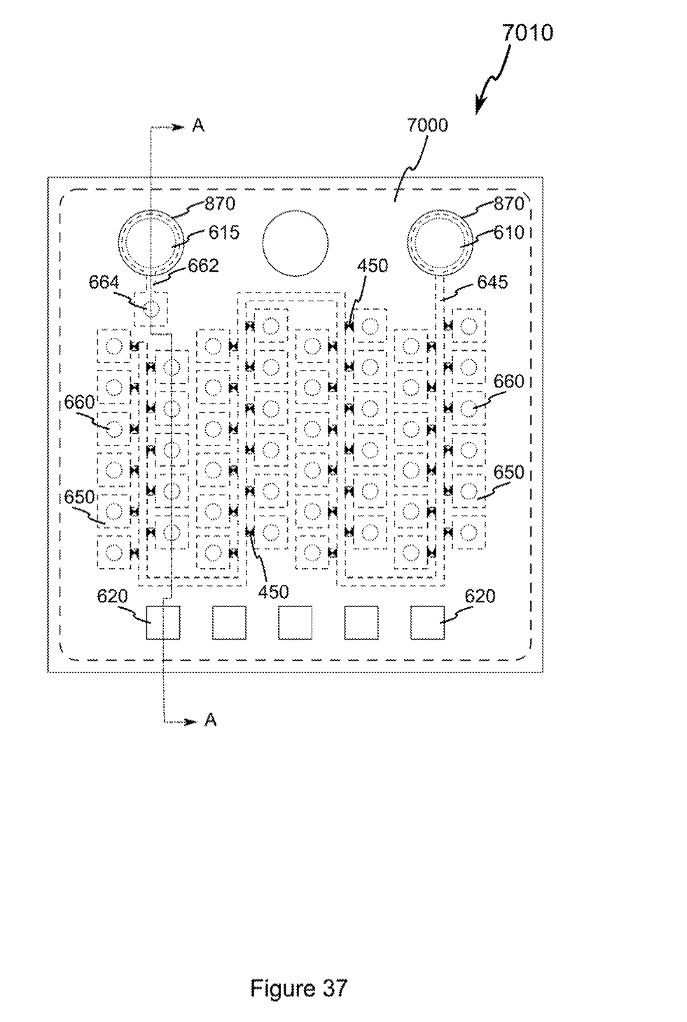

An analysis module suction port 615 is connected to a fluidic port 320 of the second syringe pump 242 via the fluidic interconnection 300. See also FIG. 37 and FIG. 38.

FIG. 37 schematically depicts the plan view of a fifth embodiment of an analysis chip 7010 incorporating the fifth embodiment of an analysis module 7000. The analysis module 7000 is one of the embodiment of analysis module used in the assay device 800. In the preferred embodiment of the invention, the analysis module 7000, being incorporated in an analysis chip 7010 is fabricated on a substrate 409 (best shown in FIG. 31) using microfabrication process steps that was described earlier in this specification. The substrate 409 comprises a layer of silicon, quartz, or other suitable material.

Similarly to the analysis module 600 and the analysis module 3500, the analysis module 7000 can perform a wide variety of fluidic, electronic, optoelectronic, and biochemical functions. In particular, the analysis module 7000 module is configured to detect the presence or absence of at least an analyte in the sample. The at least an analyte may be at least a diagnostic marker (e.g., a disease marker) and/or a prognostic marker. The at least an analyte may be one or more nucleic acids, proteins, peptides, or other biomarkers. Each of the one or more nucleic acids may be a diagnostic marker (e.g., a disease marker) and/or a prognostic marker. Each of the one or more nucleic acids may indicate a presence or an absence of a certain nucleic acid sequence, a certain gene, and/or a certain single nucleotide polymorphism (SNP) in the subject providing the sample.

As FIG. 37 depicts a simplified schematic of the analysis module 7000, only certain features of the module are shown; however, it will be appreciated that the analysis module 7000 incorporates one or more other features that include features that were previously disclosed as being incorporated in the analysis module 600 and the analysis module 3500. For example, some of the features of the analysis module 7000 that are not shown are the one or more of photosensors 710, one or more of calibration and/or control chambers 670, one or more heaters 680, but it will be appreciated that the these components play the same role in the analysis module 7000 as similarly numbered components of as that in the analysis module 600 and the analysis module 3500. The liquid sensors and the temperature sensors incorporated in the analysis module 7000 are also not shown.

As shown in FIG. 37, the analysis module 7000 incorporates a plurality of bond pads 620, an analysis module inlet 610, the analysis module suction port 615, one or more of channels 645, one or more detection chambers 650 with a roof opening 660 in its roof, and optionally a hydrogel valve 450 at the detection chamber inlet of each of the detection chambers 650 (i.e., optionally each detection chamber includes a valve 450 closable to seal the detection chamber 650). The embodiment of the analysis module 7000 that is shown in FIG. 37 includes at least 47 of the detection chambers 650. The preferred embodiments of the analysis module 7000 are fabricated on the substrate 409 (see FIG. 31). It will be appreciated that the these components play the same role in the analysis module 7000 as similarly numbered components of as that in the analysis module 600 and the analysis module 3500.

The skilled worker will appreciate that the geometric locations of many of the components of the analysis module 7000 or the analysis chip 7010 can be varied without changing the functionality of the analysis module 7000 or the analysis chip 7010; this implies that the length of electrical interconnections (not shown) or the fluidic interconnections can be varied by the skilled worker in such a way that does not change the basic functionality of the analysis module 7000 or the analysis chip 7010.

The skilled worker will also appreciate that the quantitative aspects of many of the components of the analysis module 7000 or the analysis chip 7010 can be varied to scale the capabilities of the analysis module 7000 or the analysis chip 7010 without changing the basic role of the analysis module 7000 or the analysis chip 7010, e.g., the number of the detection chambers 650 of the analysis module 7000 can be increased by the skilled worker to scale up the number of the probes the analysis module 7000 can incorporate.

FIG. 38 shows a schematic cross-section of the analysis chip assembly depicting an analysis chip suction manifold 850 attached to the analysis chip 7010. As noted previously, the fluidic port 320 of the second syringe pump 242 is connected to the analysis module suction port 615 via the fluidic interconnection 300. As shown in FIG. 38, the analysis module suction port 615 is in fluidic communication with the interior of analysis chip suction manifold 860 via a suction channel 662 and a suction channel roof opening 664, and the interior of analysis chip suction manifold 860 is, in turn, in fluidic communication with the detection chambers 650 of the analysis module 7000 via one or more of the roof openings 660 of one or more of the detection chambers 650. Consequently, the fluidic port 320 of the second syringe pump 242 is in fluidic communication with the detection chambers 650 of the analysis module 7000.

With the fluidic port 320 of the second syringe pump 242 being in fluidic communication with the detection chambers 650 of the analysis module 7000, when the second syringe pump 242 is put in the suction mode, the partial vacuum created in the detection chambers 650 of the analysis module 7000 draws the biochemical mix into the detection chambers 650.

Some embodiments of the invention utilize a single-piece variant of the analysis chip suction manifold 850. In some of the embodiments, the single-piece variant of the analysis chip suction manifold 850 is made of a polymer, e.g., PMMA or COC (cyclic olefin copolymer). In some of the embodiments, the single-piece variant of the analysis chip suction manifold 850 is micromilled from a block of polymer, e.g., PMMA or COC.

Some embodiments of the invention utilize a multi-piece variant of the analysis chip suction manifold 850. In some of the embodiments, the multi-piece variant of the analysis chip suction manifold 850 is made of a polymer, e.g., PMMA or COC (cyclic olefin copolymer). In some of the embodiments, the multi-piece variant of the analysis chip suction manifold 850 is micromilled from a block of polymer, e.g., PMMA or COC. FIG. 38 depicts a multi-piece variant of the analysis chip suction manifold 850, which incorporates an analysis chip suction manifold lid 855.

The analysis chip suction manifold 850 depicted in FIG. 38 incorporates a conduit 870 for fitting in the fluidic interconnection 300 that connects the fluidic port 320 of the second syringe pump 242 to the suction channel 662 incorporated in the analysis module 7000. The analysis chip suction manifold 850 depicted in FIG. 38 also incorporates a second conduit 870 (not shown in FIG. 38 but shown in FIG. 37) for fitting in the fluidic interconnection that connects the fluidic intersection 310 of the assay device 800 to the analysis module inlet 610 of the analysis module 7000 (see FIG. 36).

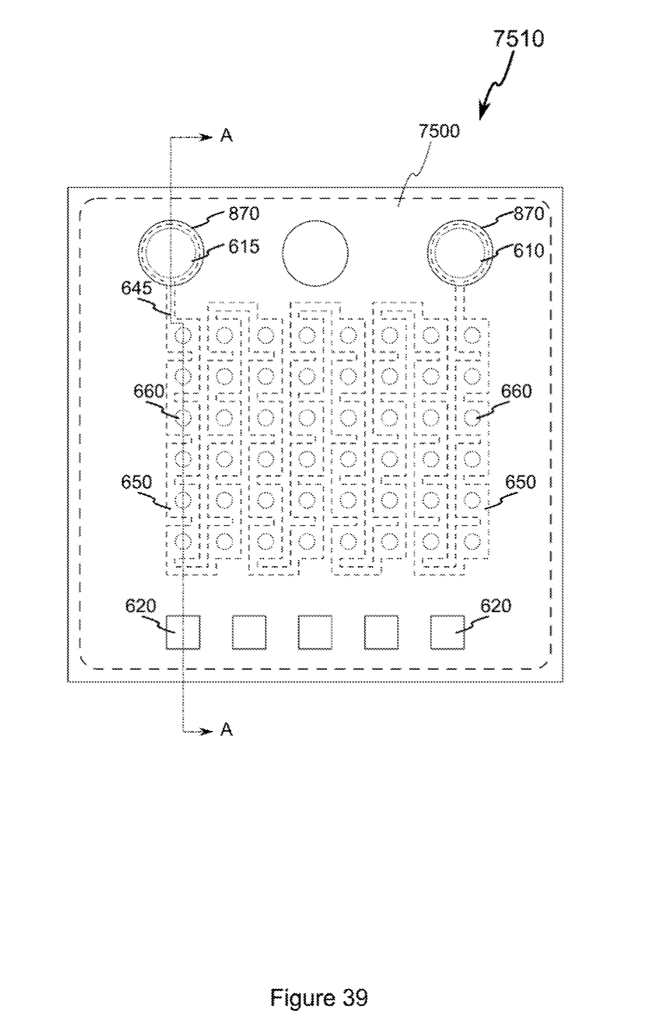

FIG. 39 and FIG. 40 show a different embodiment. FIG. 39 schematically depicts the plan view of a sixth embodiment of an analysis chip 7510, which incorporates the sixth embodiment of an analysis module 7500. The analysis module 7500 is an embodiment of analysis module optionally used in the assay device 800.

Similarly to the analysis module 600 and the analysis module 3500, the analysis module 7500 can perform a wide variety of fluidic, electronic, optoelectronic, and biochemical functions. In particular, the analysis module 7500 module is configured to detect the presence or absence of at least an analyte. The at least an analyte may be at least a diagnostic marker (e.g., a disease marker) and/or a prognostic marker. The at least an analyte may be one or more nucleic acids, proteins, peptides, or other biomarkers.

As FIG. 37 depicts a simplified schematic of the analysis module 7500, only certain features of the module are shown; however, it will be appreciated that the analysis module 7500 incorporates one or more other features that include features that were previously disclosed as being incorporated in the analysis module 600 and the analysis module 3500. For example, some of the features of the analysis module 7500 that are not shown are the one or more of photosensors 710, one or more of calibration and/or control chambers 670, one or more heaters 680, but it will be appreciated that the these components play the same role in the analysis module 7500 as similarly numbered components of as that in the analysis module 600 and the analysis module 3500. The liquid sensors and the temperature sensors incorporated in the analysis module 7500 are also not shown.

As shown in FIG. 39, the analysis module 7500 incorporates a plurality of bond pads 620, an analysis module inlet 610, the analysis module suction port 615, one or more of channels 645, and one or more detection chambers 650 with a roof opening 660 in its roof. The embodiment of the analysis module 7500 that is shown in FIG. 37 includes 47 of the detection chambers 650. The preferred embodiments of the analysis module 7500 are fabricated on the substrate 409 (see FIG. 31). It will be appreciated that the these components play the same role in the analysis module 7500 as similarly numbered components of as that in the analysis module 600 and the analysis module 3500.

The skilled worker will appreciate that the geometric locations of many of the components of the analysis module 7500 or the analysis chip 7510 can be varied without changing the functionality of the analysis module 7500 or the analysis chip 7510; this implies that the length of electrical interconnections (not shown) or the fluidic interconnections can be varied by the skilled worker in such a way that does not change the basic functionality of the analysis module 7500 or the analysis chip 7510.

The skilled worker will also appreciate that the quantitative aspects of many of the components of the analysis module 7500 or the analysis chip 7510 can be varied to scale the capabilities of the analysis module 7500 or the analysis chip 7510 without changing the basic role of the analysis module 7500 or the analysis chip 7510, e.g., the number of the detection chambers 650 of the analysis module 7500 can be increased by the skilled worker to scale up the number of the probes the analysis module 7500 can incorporate.

FIG. 40 shows a schematic cross-section of the analysis chip assembly depicting the analysis chip suction manifold 850 attached to the analysis chip 7510. As noted previously, the fluidic port 320 of the second syringe pump 242 is connected to the analysis module suction port 615 via the fluidic interconnection 300. As shown in FIG. 40, the analysis module suction port 615 is in fluidic communication with the interior of analysis chip suction manifold 860 via the channel 645 and one or more of the roof openings 660 of one or more of the detection chambers 650. The interior of analysis chip suction manifold 860 is, in turn, in fluidic communication with the detection chambers 650 of the analysis module 7500 via one or more of the roof openings 660 of one or more of the detection chambers 650. Also in parallel, the analysis module suction port 615 is in fluidic communication with the detection chambers 650 via the channel 645. Consequently, the fluidic port 320 of the second syringe pump 242 is in fluidic communication with the detection chambers 650 in addition to being in fluidic communication with the channel 645 of the analysis module 7500.

With the fluidic port 320 of the second syringe pump 242 being in fluidic communication with the detection chambers 650 and the channel 645 of the analysis module 7500, when the second syringe pump 242 is put in the suction mode, the partial vacuum created in the detection chambers 650 of the analysis module 7500 draws the biochemical mix into the detection chambers 650.

The analysis chip suction manifold 850 depicted in FIG. 40 incorporates a conduit 870 for fitting in the fluidic interconnection 300 that connects the fluidic port 320 of the second syringe pump 242 to the analysis module suction port 615 of the analysis module 7500. The analysis chip suction manifold 850 depicted in FIG. 40 also incorporates a second conduit 870 (shown in FIG. 39 but not shown FIG. 40) for fitting in the fluidic interconnection 300 that connects the fluidic intersection 310 to the analysis module inlet 610 of the analysis module 7500 (see FIG. 36).

The skilled worker in the art will readily recognize that the analysis chip suction manifold 850 can be varied, without departing from the spirit and scope of the broad inventive concept, to make variants of the analysis chip suction manifold 850 that are suitable for attachment to other embodiments of the analysis chips, e.g., the analysis chip 602 or the analysis chip 3510, to make these embodiments of the analysis chips suitable for incorporation in other embodiments of the assay device 800 in place of the analysis chip 7010 or the analysis chip 7510.

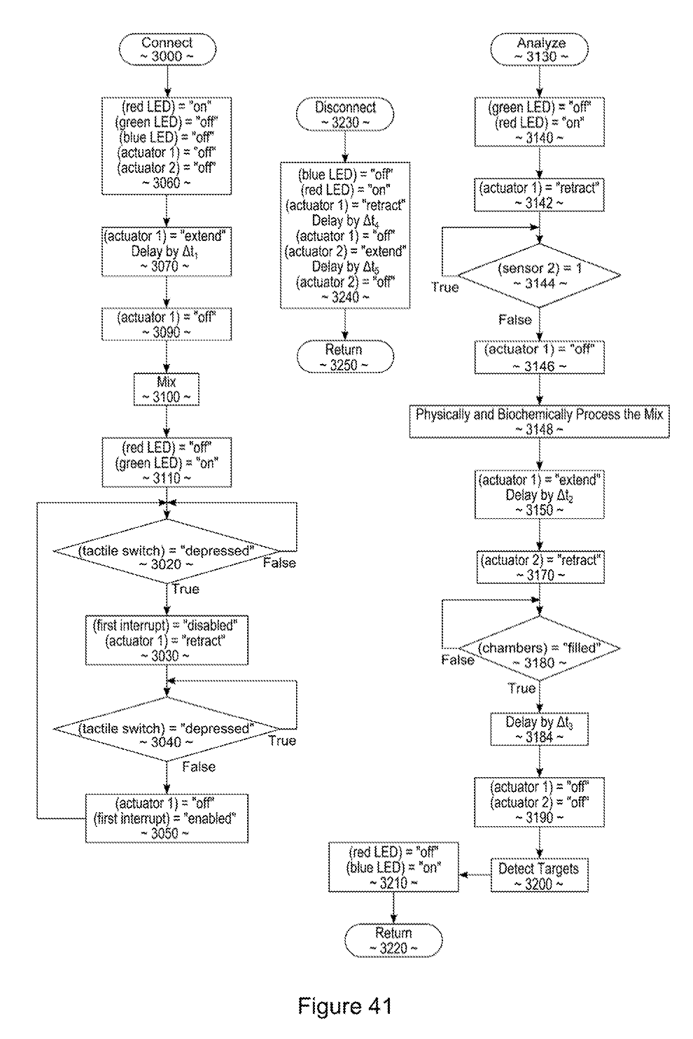

A flow diagram describing one variant, of the many variants, of the operation of the assay device 800 is shown in FIG. 41. It will be appreciated that this flow diagram only describes the more significant aspects of only one variant of the operation of the assay device 800. Aspects of the operation of the assay device that can be readily addressed by the skilled worker in the art are also left out of this description, e.g., where the volume of the collected sample reaches the maximum capacity of the assay device.

As described in the following, the operation of the assay device 800 is mostly automated, and a limited degree of user input is required in collecting and analyzing the sample.

As shown in the flow diagram, after the connection between the assay device 800 and the host is made, the control is transferred to process 3000 through an interrupt.

Following the process 3000, the assay device is initialized (commencing with process 3060). During the process 3060, the red LED 142 on the reusable block of the assay device 800 is turned on, the green LED 144 on the reusable block is turned off, the blue LED 146 on the reusable block is turned off, the linear actuator 270 driving the first syringe pump 241 is switched to the “off” state, and the linear actuator 270 driving the second syringe pump 242 is switched to the “off” state.

It should be noted that in the preferred embodiment of the invention, the assay device 800 is designed in such a way that, upon the “power-up” of the assay device 800, certain of the “signal lines” of the assay device 100 are “pulled up” or “pulled down”, as required, to put the assay device 800 in the final states that are mandated by the instructions of the process 3060, in a “glitch-free” manner, even before the signals are asserted by the controller as part of the process 3060.

After the process 3060, control is transferred to process 3070. The process 3070 comprises of the linear actuator that drives the piston of the first syringe pump 241 being set to extend followed by a time delay of Δt₁, forcing the content of the syringe barrel of the first syringe pump 241 out and through the fluidic interconnections 300, past the second liquid sensor 220, and past the breakable fluidic seal 820 into the reagent chamber 830.

In some embodiments, the content of the syringe barrel of the first syringe pump 241 comprises one or more liquid reagents 845. The one or more liquid reagents 845 can also simply be “water” or a buffer. In the assay device 800 (also in the assay device 500), the volume of the liquid reagent would, additionally, allow smaller volumes of sample, for a given fluidic “dead volume”, to be collected and analyzed. The volume of the liquid reagent would additionally allow larger fluidics with larger fluidic dead volumes, for a given sample volume to be used. The volume of the liquid reagent would additionally allow smaller volumes of sample and larger fluidics with larger fluidic dead volumes to be used.

The breakable fluidic seal 820, if present, has the role of isolating the content of the first syringe pump 241 from the one or more assay reagents 840 before use of the assay device 800.

After the completion of the process 3070, the linear actuator that drives the piston of the first syringe pump 241 is turned off (process 3090). The time delay Δt₁ is the length of time that is required for a suitable volume of the one or more liquid reagents 845 that are stored in the first syringe pump 241 to be forced out and into the reagent chamber 830 to mix with the one or more assay reagents 840. In one embodiment, the process 3070 is a “timed” process, where the time delay of Δt₁ is predetermined in such a way for the suitable volume of the one or more liquid reagents 845 to be forced out of the first syringe pump and into the reagent chamber 830. In other embodiments, the process 3070 is not a timed process, and instead other techniques are used in assuring the suitable volume of the one or more liquid reagents 845 is forced out of the first syringe pump and into the reagent chamber 830. In one embodiment, the technique that is used to assure the suitable volume of the one or more liquid reagents 845 is forced out of the first syringe pump and into the reagent chamber 830 comprises a “positional sensor” that is “linked” to the linear actuator that drives the piston of the first syringe pump 241. The output of the positional sensor is used to provide feedback to suitably control the volume of the one or more liquid reagents 845 that is forced out of the first syringe pump and into the reagent chamber 830. In another embodiment, the technique that is used to assure the suitable volume of the one or more liquid reagents 845 is forced out of the first syringe pump and into the reagent chamber 830 comprises the first liquid sensor 210; in this embodiment, the linear actuator that drives the piston of the first syringe pump 241 is turned off when the first liquid sensor 210 indicates the presence of liquid in the fluidic interconnections 300 that connect the second fluidic port 320 of the reagent chamber 830 and the fluidic intersection 310. This technique works to suitably control the volume of the one or more liquid reagents 845 that is forced out of the first syringe pump and into the reagent chamber 830.

At this stage, preferably, a mixing process 3100 is performed to better mix the one or more assay reagents 840 with the one or more liquid reagents 845. The mixing process 3100 comprises of one or more “cycles”. Each of the one or more cycles comprises of two stages. The first stage of the cycle comprises of the linear actuator that drives the piston of the first syringe pump 241 being set to retract, drawing the mix of the one or more assay reagents 840 with the one or more liquid reagents 845 into the syringe barrel of the first syringe pump 241 (i.e., the first syringe pump 241 is put in the suction mode) until the second liquid sensor 220 detects a depletion of the mix of the one or more assay reagents 840 with the one or more liquid reagents 845 in the interior of the sensor. At this stage, the mix of the one or more assay reagents 840 with the one or more liquid reagents 845 would “substantially” be in the syringe barrel of the first syringe pump 241. The second stage of the cycle comprises of the linear actuator that drives the piston of the first syringe pump 241 being set to extend, forcing the mix which includes the mix of the one or more assay reagents 840 with the one or more liquid reagents 845 out and through the fluidic interconnections 300, past the second liquid sensor 220, past the breakable fluidic seal 820 into the reagent chamber 830, and out through the fluidic interconnection 300 until the biochemical mix or the chemical mix (as defined previously in this specification) reaches the first liquid sensor 210. Upon the first liquid sensor 210 indicating the presence of liquid in the fluidic interconnections 300 that connect the second fluidic port 320 of the reagent chamber 830 and the fluidic intersection 310, the cycle gets repeated. The mixing cycle gets repeated a set number of times to assure the required level of mixing quality is achieved. During the last mixing cycle, the first syringe pump 241 is turned off when the first liquid sensor 210 indicates the presence of liquid in the fluidic interconnections 300 that connect the second fluidic port 320 of the reagent chamber 830 and the fluidic intersection 310. Optionally, with additional steps being added to the process 3100, the mixing quality can be improved by appropriately heating the biochemical mix inside the syringe barrel of the first syringe pump 241 via the heating sleeve 280 that surrounds the syringe barrel.

After this, the red LED 142 on the assay device reusable block is turned off and the green LED 144 on the assay device reusable block is turned on, signaling to the user that the procedure for the collection of the sample can commence (process 3110).

For the NAF sample, the NAF is aspirated according to one of the standard NAF aspiration procedures.

To collect the sample, the user brings the inlet 810 of the assay device 800 into contact with the aspirated NAF (or the liquid sample of a different type) and depresses the tactile switch 150.

Process 3020 monitors the status of the tactile switch 150. If the tactile switch is detected as “released”, i.e. “(tactile switch)=“depressed”” is a false statement, the control stays in a waiting and monitoring loop. If the tactile switch is detected as “depressed”, i.e. “(tactile switch)=“depressed”” is a true statement, the control is transferred to process 3030.

Process 3030 comprises of a “first interrupt” being disabled (the reason for this instruction will be clarified in the subsequent processes) and the linear actuator that drives the piston of the first syringe pump 241 being set to retract, drawing the sample through the inlet 810 and through the fluidic interconnections 300 in the direction defined, in order of the flow of the sample, by the fluidic intersection 310, the first liquid sensor 210, the reagent chamber 830, the breakable fluidic seal 820, and the second liquid sensor 220 towards the syringe barrel of the first syringe pump 241. Through this process, the sample will eventually follow the mix of the one or more assay reagents 840 with the one or more liquid reagents 845, through the fluidic intersection 310, past the first liquid sensor 210, through the reagent chamber 830, past the breakable fluidic seal 820, and past the second liquid sensor 220 into the syringe barrel of the first syringe pump 241.

Process 3020 is followed by process 3040, which again monitors the status of the tactile switch 150. If the tactile switch is detected as “depressed”, i.e. “(tactile switch)=“depressed”” is a true statement, the control flow continues to monitor the status of the tactile switch 150, while at the same time allows the sample to continue to be drawn towards, and eventually into, the syringe barrel of the first syringe pump 241. If the tactile switch is detected as “released”, i.e. “(tactile switch)=“depressed”” is a false statement, the control is transferred to process 3050.

The process 3050 comprises of turning the linear actuator that drives the piston of the first syringe pump 241 to the “off” state, i.e. pausing the collection of the sample, and enabling the first interrupt, allowing an “interrupt-driven” analysis phase of the sample to be commenced.

At the completion of the process 3050, the control is transferred back to the process 3020, where the status of the tactile switch 150 is again monitored. If the tactile switch is detected as “depressed”, the control is again transferred to process 3030, and the collection of more of the sample will proceed as described in the previous paragraphs.

The skilled worker will appreciate that the process 3020 and the process 3040 can be replaced with interrupt-driven processes, where the “depressed” and the “released” states of the tactile switch triggers “interrupt service routines” that control the first syringe pump 241 in such a manner as to draw the sample or pause the collection of the sample as desired.

Referring back to the process 3030, after enabling the first interrupt during the process 3050, the window for commencing the interrupt-driven analysis phase of the sample will last until the disabling of the first interrupt during the execution of the process 3030. However, when it is desired to commence the interrupt-driven analysis phase of the sample, naturally no more of the sample would be collected and the control would stay within the process 3020 and will not reach the instruction in the process 3030 that disables the first interrupt.

After the desired amount of the sample has been collected, the analysis phase can commence. The analysis phase of the assay device operation begins by the user preferably replacing the protective cap of the assay device expendable block 120 and giving the appropriate “analyze” command to the assay device. This command can be given via a command being initiated via the user interface of the assay device application software on the host. As mentioned previously, the analyze command is interrupt-driven, and it interrupts the control flow, transferring control to process 3130 in the flow diagram.

The analysis of the sample commences according to process 3140 in the flow diagram where the green LED 144 is turned off, indicating to the user that no more collection of the sample is to be attempted, and the red LED 142 is turned on.

Process 3142 follows the process 3140. Process 3142 comprises of the linear actuator that drives the piston of the first syringe pump 241 being set to retract, drawing the biochemical mix or the chemical mix of the sample, the one or more assay reagents 840, and the one or more liquid reagents 845 into the syringe barrel of the first syringe pump 241. Drawing of the biochemical mix or the chemical mix continues until the second liquid sensor 220 detects a depletion of the biochemical mix or the chemical mix in the interior of the sensor (process 3144 of the flow diagram). At this stage, the linear actuator that drives the piston of the first syringe pump 241 is set to the “off” state (process 3146 of the flow diagram).

At this stage, the biochemical mix or the chemical mix of the sample, the one or more assay reagents 840, and the one or more liquid reagents 845 would “substantially” be in the syringe barrel of the first syringe pump 241.

The biochemical mix now undergoes a number of physical and biochemical processes (depicted as process 3148 in the flow diagram). These processes take place by appropriately heating the biochemical mix inside the syringe barrel of the first syringe pump 241 via the heating sleeve 280 that surrounds the syringe barrel. In effect, the syringe barrel additionally functions as a reaction chamber.

The physical and biochemical processes that take place in the reaction chamber (the syringe barrel of the first syringe pump 241, in this embodiment) include the potential further mixing of the biochemical mix, the lysis of any cells in the biochemical mix to release their genetic material, and the amplification of various desired segments of this genetic material. The genetic material includes one or more nucleic acid molecules, which include one or more of DNA molecules, RNA molecules, and their methylated forms. The assay device is configured to amplify a nucleic acid or part thereof in the reaction chamber preferably via an isothermal amplification reaction. The biochemical mix wherein the genetic material in the biochemical mix has undergone amplification is called the amplicon.

The physical and biochemical processing of the biochemical mix is a timed process. After the amplification of the genetic material in the biochemical mix is completed, control is transferred to process 3150.

Process 3150 comprises of the linear actuator that drives the piston of the first syringe pump 241 being set to extend followed by a time delay of Δt₂, forcing the biochemical mix (amplicon) out and through the fluidic interconnections 300, past the second liquid sensor 220, past the breakable fluidic seal 820, through the reagent chamber 830, past the first liquid sensor 210, and through the fluidic intersection 310 towards the inlet 810.

The time delay Δt₂ is the length of time that is required for the biochemical mix to reach the fluidic intersection 310 or, more preferably, for a suitable volume of the biochemical mix to be expelled out of the inlet 810. The purpose of expelling the suitable volume of the biochemical mix out of the inlet 810 is to dispose of the leading slug of biochemical mix that would have constituted the volume of biochemical mix that existed between the second liquid sensor 220 and the fluidic port 320 of the first syringe pump 241 during the physical and biochemical processing. The said slug of biochemical mix, being located outside of the syringe barrel of the first syringe pump 241, would have not properly undergone the physical and biochemical processing, and it is advantageous to dispose of this slug.

In the embodiment just described, the process 3150 is a “timed” process, where the time delay of Δt₂ is predetermined in such a way for the biochemical mix to reach the fluidic intersection 310 or, more preferably, for a suitable volume of the biochemical mix to be expelled out of the inlet 810. In other embodiments, the process 3150 is not entirely a timed process, and, instead, other techniques are used in assuring the suitable volume of the biochemical mix is expelled out of the inlet 810. In one embodiment, the technique that is used to assure the suitable volume of the biochemical mix is expelled out of the inlet 810 comprises a “positional sensor” that is “linked” to the linear actuator that drives the piston of the first syringe pump 241. The output of the positional sensor is used to provide feedback to suitably control the volume of the biochemical mix that is forced out of the first syringe pump to assure the suitable volume of the biochemical mix is expelled out of the inlet 810. In another embodiment, the technique that is used to assure the suitable volume of the biochemical mix is forced out of the first syringe pump 241 comprises the first liquid sensor 210; in this embodiment, the first syringe pump 241 continues to force the biochemical mix out for an additional suitably determined time interval after the first liquid sensor 210 indicates the presence of liquid in the fluidic interconnections 300 that connect the second fluidic port 320 of the reagent chamber 830 and the fluidic intersection 310. This technique works to suitably control the volume of the biochemical mix that is expelled out of the inlet 810.

After the completion of the process 3150, the linear actuator that drives the piston of the second syringe pump 242 is set to retract (process 3170).

The retraction of the linear actuator that drives the piston of the second syringe pump 242 at a suitable rate compared to the extension of the linear actuator that drives the piston of the first syringe pump 241 draws the biochemical mix through the analysis module inlet 610, into the analysis module. Enough of the biochemical mix is drawn into the analysis module 7000 to fill the detection chambers 650 that are incorporated in the analysis module 7000. The method by which the filling of the detection chambers 650 of the analysis module 7000 is determined relies on one or more liquid sensors 200 that are located in the flow path in the analysis module 7000 in such a way as to be able to ascertain the filled state of the detection chambers 650 in the analysis module 7000. After a suitable time interval after the liquid sensors 200 detect the presence of liquid in the “sensors' manifolds”, it can be assumed that the detection chambers 650 are filled; therefore, the actions of the first syringe pump 241 and the second syringe pump 242 continue to draw the biochemical mix into the detection chambers 650 of the analysis module 7000 until after the liquid sensors 200 have indicated the presence of the liquid (depicted as process 3180 in the flow diagram) and after the suitable time delay Δt₃ a after the detection of the liquid has expired (depicted as process 3184 in the flow diagram).

At this stage, as depicted as process 3190 in the flow diagram, the first syringe pump 241 and the second syringe pump 242 are turned off, and the detection of the various target molecules (target nucleic acid sequences or various target proteins), contained in the biochemical mix, can now commence (process 3200).

Each of the detection chambers 650 in the analysis module 7000 includes a probe specific to a particular analyte. The analyte may be a nucleic acid sequence. Each of the detection chambers 650 includes an electrochemiluminescence resonance energy transfer probe that is anchored to an interior surface of the detection chamber 650. For the purpose of this patent application, the chemical mix or the biochemical mix forced into the detection chambers 650 and the electrochemiluminescence resonance energy transfer probe will together be referred to as the contents of the detection chambers 650. The analysis module 7000 comprises a plurality of heaters 680 for heating the contents of the detection chambers 650. When a nucleic acid sequence in the biochemical mix is complementary to the electrochemiluminescence resonance energy transfer probe in a detection chamber 650, the nucleic acid sequence hybridizes with the electrochemiluminescence resonance energy transfer probe and forms a hybrid, indicating the presence of a target molecule (a target nucleic acid sequence).

Electrical excitation of the content of the detection chambers 650 in parallel to the detection of any emission by the electrochemiluminescence resonance energy transfer probes in the detection chambers is used to detect the existence of hybrids in each of the detection chambers 650.

Each of the detection chambers 650 incorporates two excitation electrodes 690, an anode and a cathode. Additionally, the analysis chip comprises a plurality of photosensors 710 adjacent to the detection chambers 650 for detecting any emission by the electrochemiluminescence resonance energy transfer probes in the detection chambers 650. An excitation current is forced between the two electrodes 690 in each of the detection chambers 650, and any electrochemiluminescence emission by the electrochemiluminescence resonance energy transfer probe is detected by the photosensors 710 adjacent to the detection chambers 650. Under this arrangement, the detection of the emission by the electrochemiluminescence resonance energy transfer probes in the detection chambers 650 corresponds to the detection of the nucleic acid sequences complementary to the probes that have been loaded in the detection chambers.

The hybridization data gathered, in the manner described, is further processed by the host and compared to the database of the diagnostic markers (e.g., disease markers) and/or prognostic markers to meet the diagnostic and prognostic objectives (process 3200).

At this stage, with the detection of the presence of the diagnostic markers and/or prognostic markers having been completed, the host turns the red LED 142 off and the blue LED 146 on, indicating to the user that the analysis results are ready. This is depicted as process 3210 in the flow diagram. In parallel to the indication given to the user by the blue LED 146, the user interface of the application software running on the host also provides an indication to the user of the completion of the detection and analysis process. At this stage, the user can use the user interface of the application software on the host to view or further process the results of the analysis of the sample.

After the completion of the process 3210, process 3220 returns the control to higher-level host software.

At this stage, the user may remove the expendable block of the assay device 800 and dispose of it accordingly. Removing the expendable block triggers an interrupt, which transfers the control to process 3230, which in turn transfer control to process 3240.

As indicated in the flow diagram, during the execution of the process 3240, the blue LED 146 is turned off, the red LED 142 is turned on, the linear actuator 270 that drives the first syringe pump 241 is set to retract, and after a time delay of Δt₄, the linear actuator 270 that drives the first syringe pump 241 is set to the off state, and the linear actuator 270 that drives the second syringe pump 242 is set to extend, and after a time delay of Δt₅, the linear actuator 270 that drives the second syringe pump 242 is set to the off state. The time delay Δt₄ and the time delay Δt₅ are determined by the application software in such a way that the linear actuator 270 that drives the first syringe pump 241 is suitably retracted and the linear actuator 270 that drives the second syringe pump 242 is suitably extended to make the reusable block of the assay device 800 ready for receiving an unused unit of the expendable block for another analysis cycle, if desired. Then, as indicated in process 3250, the control is returned to higher-level application software, and the diagnostic, prognostic, and analytical system waits for any desired further use.

In the embodiment just described, the process 3240 is a “timed” process, where the time delay Δt₄ and the time delay Δt₅ are determined by the application software in such a way that the linear actuator 270 that drives the first syringe pump 241 is suitably retracted and the linear actuator 270 that drives the second syringe pump 242 is suitably extended to make the reusable block of the assay device 800 ready for receiving an unused unit of the expendable block. In other embodiments, the process 3240 is not a timed process, and instead other techniques are used in assuring that the linear actuator 270 that drives the first syringe pump 241 is suitably retracted and the linear actuator 270 that drives the second syringe pump 242 is suitably extended to make the reusable block of the assay device 800 ready for receiving an unused unit of the expendable block. In one embodiment, the technique that is used to assure that the linear actuator 270 that drives the first syringe pump 241 is suitably retracted and the linear actuator 270 that drives the second syringe pump 242 is suitably extended to make the reusable block of the assay device 800 ready for receiving an unused unit of the expendable block comprises a first “positional sensor” that is “linked” to the linear actuator 270 that drives the first syringe pump 241 and a second “positional sensor” that is “linked” to the linear actuator 270 that drives the second syringe pump 242. The outputs of the first positional sensor and the second positional sensor are used to provide feedback to suitably control the linear actuator 270 that drives the first syringe pump 241 is suitably retracted and the linear actuator 270 that drives the second syringe pump 242 is suitably extended to make the reusable block of the assay device 800 ready for receiving an unused unit of the expendable block.

As previously described, the assay device 800 includes a communication interface that permits communication between the assay device and a processing system; the processing system may be the host 8010. In many of the embodiments of the assay device 800, a USB link is used for connecting the assay device 800 to the host 8010 (see FIG. 45). A standard USB cable 110 is preferably used for this purpose. It will be appreciated that in other examples, the assay device may communicate wirelessly (e.g., via Wi-Fi, Bluetooth, etc.) with the host/processing system. The assay device generates one or more signals indicative of the presence and/or absence of at least one analyte, and the one or more signals are received by a processing system (i.e. the host 8010).

Analysis of Biopsied Tissue Samples with the Assay Device

Biopsied tissue samples can be analyzed by the current invention. The method involves using standard methods to obtain liquid tissue extracts or liquid cell lysates from the tissue samples, and then, using, e.g., the assay device 800 to analyze the liquid tissue extracts or the liquid cell lysates.

### A Fourth Embodiment of Assay Device

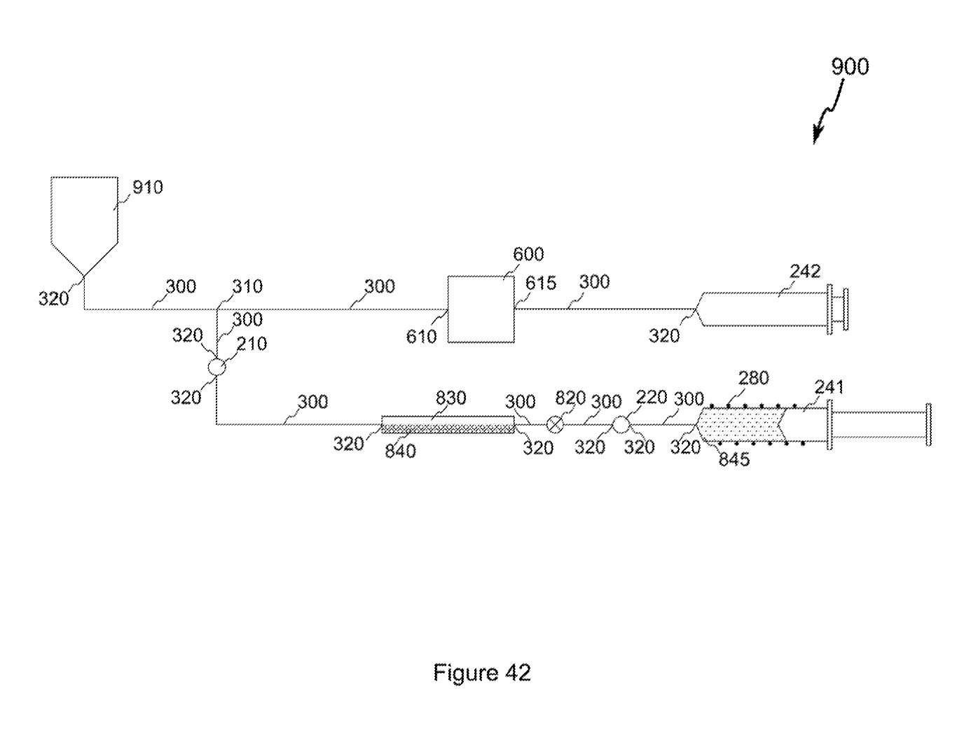

An assay device 900 will be described next. The assay device 900 is a fourth embodiment of the assay device, and it is shown schematically in FIG. 42. The assay device 900 utilizes a tissue-preprocessing receptacle 910 for receiving a tissue sample or a liquid sample. The samples can comprise, e.g., biopsied tissue samples or a tissue aspirate obtained via one of the standard fine needle aspiration biopsy procedures. The tissue-preprocessing receptacle 910 can also receive various chemicals, buffers or reagents, e.g., lysing reagents. The tissue-preprocessing receptacle 910 automatically, semiautomatically, or manually preprocesses the tissue samples to obtain liquid tissue extracts or liquid cell lysates for the purpose of analysis.

Functionally, except for the tissue-preprocessing receptacle 910, the assay device 900 is very similar to the assay device 800. The functional difference between the assay device 800 and the assay device 900 is that a fluidic interconnection 300 connects a first fluidic intersection 310 of the assay device 900 to a fluidic port 320 of the tissue-preprocessing receptacle 910, whereas in the assay device 800, the fluidic interconnection 300 connects the first fluidic intersection 310 of the assay device 800 to the inlet 810 of the assay device 800.

### Some Other Embodiments of Assay Device

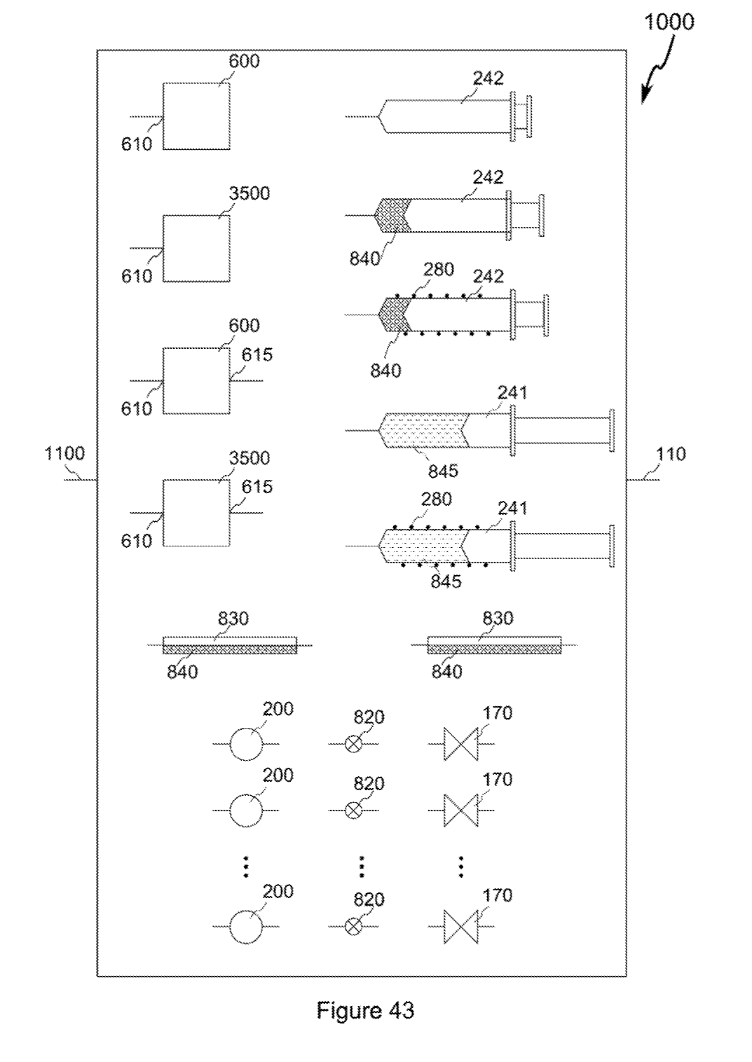

The skilled worker will appreciate that many other embodiments of the assay devices can be realized as an assay device 1000 (shown in FIG. 43) that incorporate one or more of a sample induction components 1100, e.g., the sample inlets, or the tissue-preprocessing receptacle, a number of the syringe pumps 240, a number of the valves, a number of the analysis modules of one or more embodiments of the analysis modules disclosed in this specification, a number of the sensors, and a number of the interconnections between these components where these numbers may be different from the number of these components in the embodiments of the assay device previously disclosed in this specification. The USB link used for connecting the assay device to the host is also shown.

The system and its components described above are purely illustrative and the skilled worker in the art will readily recognize many variations and modifications, which do not depart from the spirit and scope of the broad inventive concept.

### Liquid Sensors

Referring to FIG. 8, FIG. 11, and FIG. 15, the liquid sensors 200 will be described next. The liquid sensors 200 are “conductive liquid sensors” (see FIG. 15). The liquid sensor 200 comprises a plurality of electrodes 690. Presence of the liquid in the “sensor manifold” and any subsequent bridging between the liquid and any two of the electrodes 690 increases the electrical conductivity between the said two of the electrodes, indicating the presence of liquid in the sensor manifold.

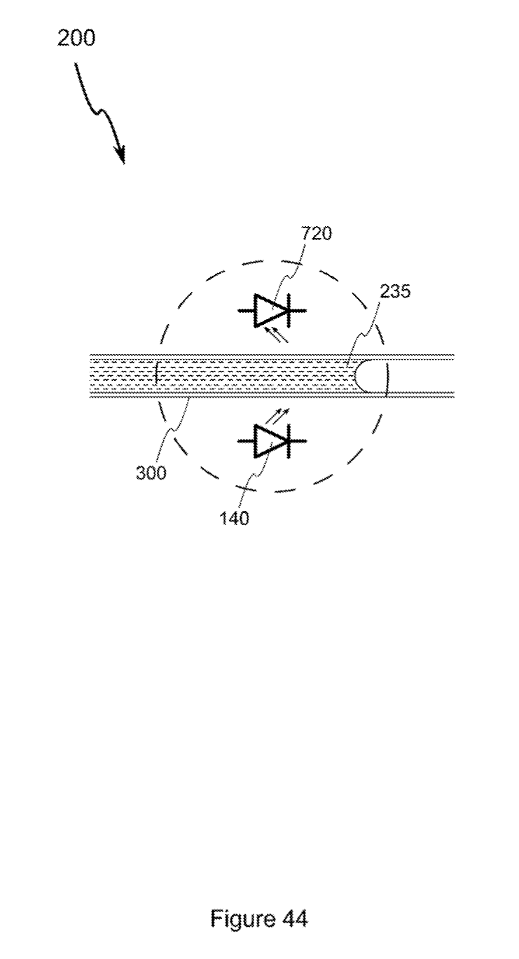

Referring to FIG. 6, FIG. 12, FIG. 13, FIG. 14, FIG. 32, FIG. 36, FIG. 42, and FIG. 44, the first liquid sensor 210, the second liquid sensor 220, and the third liquid sensor 230 will be described next.

In some embodiments of the invention, one or more of the first liquid sensor 210, the second liquid sensor 220, and the third liquid sensor 230 are “conductive liquid sensors” that function in the same manner as the liquid sensor 200.

In some other embodiments of the invention, one or more of the first liquid sensor 210, the second liquid sensor 220, and the third liquid sensor 230 are “optical liquid sensors” that will be described next. A depiction of the optical liquid sensor is given in FIG. 44. The optical liquid sensor comprises a light source (e.g., an LED 140 emitting at a preferred “wavelength”) and a photodetector (e.g., a photodiode 720) straddling a fluidic interconnection 300. The optical liquid sensor also comprises “drive” circuitry associated with the light source and analog and digital pick-up circuitry for picking up any signals generated by the photodetector.

During operation, output of the LED 140 is absorbed differently by the fluidic interconnection 300 with a liquid 235 present in it from when no liquid is present in the fluidic interconnection 300. The photodiode 720 and the pick-up circuitry detect the change in the absorbed output of the LED 140, and subsequently determine the presence or the absence of liquid in the fluidic interconnection 300.

It should be emphasized that, in this specification, where the optical liquid sensor is referenced, it may refer to such configurations where the optical liquid sensor is connected to the other components of the assay device by connecting the fluidic interconnection 300 of the optical liquid sensor to the other components of the assay device via fluidic interconnections 300 that are additional to the fluidic interconnection 300 of the optical liquid sensor. Alternatively, a reference to the optical liquid sensor may refer to such configurations where the LED 140 and the photodiode 720 are positioned around one of the fluidic interconnections 300 that would have already existed in the assay device even in the absence of the optical liquid sensor that is being referenced.

### Assay Chemistry

A number of markers are currently known for breast cancer and other diseases. There is a good possibility that additional markers for the testing of breast cancer and other diseases will become known in the future. The present invention is for detecting multiple markers, as well as single markers, for breast cancer and other diseases, but the following description emphasizes the application of the invention for breast cancer testing.

Estrogen receptor/p53: The ESR1 gene codes for estrogen receptor alpha (ERα), a nuclear transcription factor that is activated by estrogen. Although activation regulates growth and differentiation of both normal and cancerous breast epithelial cells, the differentiating factor is that in breast cancers ESR1 has been reported to be amplified leading to a high expression of ERα. However, there has been much debate over whether this gene amplification is a reliable indicator, and the consensus appears to veer away from its acceptance with many claiming that the frequency of amplification is usually very low (Ooi, et al., 2012).

However, ER⁻/ER⁺ tumor subtypes are very important indicators of prognosis;

therefore, provision for testing for these subtypes is included on the marker panel (see Receptor Status by PCR below). In general, an ER⁻tumor is considered to be “high grade” with several mutations in the TP53 tumor suppressor gene. Expression of the TP53 gene is important for producing a tumor suppressor protein p53 that intervenes in tumor growth by inducing apoptosis of genetically altered cells. The normal p53 protein is transient and degrades very quickly whereas the abnormal p53 protein accumulates and fails to act on tumor cells. Detection of the p53 protein is a key indicator of positive (abnormal) p53 status and from a clinical perspective assists with indicating responses to chemotherapy and ultimately prognosis (Varna, Bousquet, Plassa, & Bertheau, 2011).

The reliability of TP53 status alone has been questioned and more recent research has shown that it is necessary to look at TP53 status and ER status together to get a meaningful prognosis. Thus, a poor prognosis is predicted when both TP53 and ER are positive whereas a more favorable prognosis is indicated by positive TP53 status and negative ER status. Options are to test against a p53 signature comprising several genes affected by transcription of TP53. This is considered to be more reliable than direct testing for TP53 mutations. The use of Affymetrix Gene-Chip microarrays has been employed for this purpose (Coates, et al., 2012).

Different isoforms of p53 are also significant in terms of prognosis (Coates, et al., 2012). The β-isoform is associated with ER expression whereas the γ-isoform is associated with TP53 mutation. Thus, a poor prognosis is predicted with expression of abnormal p53 but not p53γ and a more favorable prognosis is implicated by expression of abnormal p53 and p53γ.

It is clear that these subtleties in diagnosis require the simultaneous detection of multiple markers.

Methods for detection of abnormal p53 are immunohistochemistry, FASAY testing, and direct sequencing of the TP53 gene. The last two methods are considered to be the most reliable with direct sequencing being applicable to the assay devices, which are the subject of this invention.

Genetic testing relevant to this application is exemplified below.

Primers for ESR1: In one example, qPCR is performed in triplicate using combinations of primer pairs and TaqMan probes targeting genetic sequences in the ESR1 (exon 1) and ESR2 (exon 5) genes. The sequences of PCR primers and hybridization probes are listed below. The PCR program included a 10-min denaturation at 94° C. followed by 40 cycles of 15 sat 72° C. and 1 min at 60° C. The nonamplified ESR2 gene served as an internal control for the normalization of ESR1 PCR products. Relative quantification results were calculated according to the ΔΔCt method. (Hoist, et al., 2007)

Primers/Probes used for ESR1 qPCR:
ESR1 forward primer:
GCCAACGCGCAGGTCTA
ESR1 reverse primer:
GCCGCAGCCTCAGA
ESR1 hybridization probe:
FAM-CTCCCCTACGGCCCC-NFQ
ESR2 hybridization probe:
FAM-CTGGACGCCCTGAGCC-NFQ

ESR1 sequence analysis: In one example, four breast cancers showing ESR1 amplification and four breast cancers with normal copy numbers (same as for qPCR analysis) were selected for ESR1 mutation analysis. DNA was extracted according to Qiagen's protocols. All ESR1 exons (1-8) were amplified via a single multiplex PCR and sequenced directly using the BigDye Terminator Cycle Sequencing Ready Reaction Kit (Applied Biosystems). Primers used for PCR and sequencing are listed below. Sequencing products were analyzed on an ABI Prism 3100 Genetic Assay device (Applied Biosystems). (Hoist, et al., 2007)

Primers used for ESR1 sequence analysis:
Ex1a forward primer,
CTC TTT TTC CAG GTG GCC CG
Ex1a reverse primer,
TAG ACC TGC GCG TTG GCG G
Ex1b forward primer,
GAG GGC GCC GCC TAC GAG TT
Ex1b reverse primer,
GGG CGC GGG TAC CTG TAG A
Ex1c forward primer,
GGT GCC CTA CTA CCT GGA GA
Ex1c reverse primer,
TAG GCT CTC CCT TCT CCC TC
Ex2 forward primer,
CCA GAG AGT GCA TGT TTT GC
Ex2 reverse primer,
GGA TCT GCT CAT AGG ATC AAA A
Ex3 forward primer,
AGA TTC TGA CTG GCT AAG TTT CC
Ex3 reverse primer,
TGG TGT TAT TCC AAT GGG TAG AG
Ex4a forward primer,
TTT TTT CCA CCT GTG TTT TC
Ex4a reverse primer,
CTT AGA GCG TTT GAT CAT GA
Ex4b forward primer,
GTG AAG TGG GGT CTG CTG GA
Ex4b reverse primer,
AGC TTC ACT GAA GGG TCT GG
Ex4c forward primer,
CCG ACC AGA TGG TCA GTG CCT T
Ex4c reverse primer,
TCT TAA AAG CTG CGC TTC GC
Ex5 forward primer,
ATG AGT CTT TTT CAT TTG AGT CAG C
Ex5 reverse primer,
TGA ACT ATG ATC GTA AAG AAC ATG C
Ex6 forward primer,
GAA CCC TTT CAT GTC TTG TGG
Ex6 reverse primer,
GGG TAG ATC GTA TCT GGT TGA A
Ex7 forward primer,
GGT CTC CTA GAC CTC ATC CTC TT
Ex7 reverse primer,
GGG GGC ATG TTT TCT TTA TG
Ex8 forward primer,
AAA GTA GTC CTT TCT GTG TCT TCC C
Ex8 reverse primer,
GGG ATT ATC TGA ACC GTG TGG

Primers for TP53: Approximately 10 mg of tumor tissue (>40% of tumor cells) was homogenized in 750 μL of QIAzol lysis reagent (Qiagen Ltd, Crawley, West Sussex, UK), and total RNA was extracted (Qiagen). RNA quality was assessed using the BioAssay device 2100™ (Agilent Technologies, Palo Alto, Calif., USA) prior to reverse transcription polymerase chain reaction (RT-PCR) analysis, and all samples with a 28S:18S ratio of less than 1.2 were discarded. Reverse transcription was performed with 0.5 μg of total RNA using the Cloned AMV Reverse Transcription Kit (Invitrogen, Paisley, UK), and cDNA quality was confirmed by PCR amplification of the actin reference gene. Samples for which actin could not be amplified after 30 cycles of PCR were discarded. p53 isoform cDNA was amplified by two consecutive PCR assays (nested PCR) of 30 cycles each, and the PCR primers used were specific for each of the p53 isoforms analyzed. The different primers used and their corresponding sequences are listed in Table 1. For each p53 isoform, the nested PCR assay was performed as two consecutive PCR reactions (I, II) with two separate primer pairs as indicated. (F): Forward, (R): Reverse. (Bourdon, et al., 2011).

TABLE 1

| | | Primer name | |
|---|---|---|---|
| | | (targeted | |
| mRNA | PCR | region) | 5′-3′ sequence |
| p53 | I | e2.1 (F) (exon2) | GTCACTGCCATGGAGGAGCCGCA |
| | | RT1 (R) (exon11) | GACGCACACCTATTGCAAGCAAGGGTTC |
| | II | e2 (F) (exon2) | ATGGAGGAGCCGCAGTCAGAT |
| | | RT2 (R) (exon11) | ATGTCAGTCTGAGTCAGGCCCTTCTGTC |
| p53β | I | e2.1 (F) (exon2) | GTCACTGCCATGGAGGAGCCGCA |
| | | RT1 (R) (exon11) | GACGCACACCTATTGCAAGCAAGGGTTC |
| | II | e2 (F) (exon2) | ATGGAGGAGCCGCAGTCAGAT |
| | | p53β (R) (exon9b) | TTTGAAAGCTGGTCTGGTCCTGA |
| p53γ | I | e2.1 (F) (exon2) | GTCACTGCCATGGAGGAGCCGCA |
| | | RT1 (R) (exon11) | GACGCACACCTATTGCAAGCAAGGGTTC |
| | II | e2 (F) (exon2) | ATGGAGGAGCCGCAGTCAGAT |
| | | p53γ (R) (exon9b) | TCGTAAGTCAAGTAGCATCTGAAGG |

To determine mutation status of the gene for p53γ, the entire open reading frame of the isoform was sequenced using the Sanger method (BigDye Terminator, ABI 3730 Genetic Analyser—Applied Biosystems, Warrington, UK) with the primers JWF (5′-AGCCAAGTCTGTGACTTGCA) and MP9ER (5′-TCTCCCAGGACAGCACAAACACG). [Check!]

BRCA gene mutation: Four BRCA tumor suppressor genes have been identified to date: BRCA1 (Chromosome 17), BRCA2 (Chromosome 13), BRCA3 (Chromosome 11) and BRCA4 (Chromosome 13). Mutations in these genes point to an increased risk of a patient getting cancer. However, mutations are only detected in patients with a family history, and because the genes are quite large and the positions of mutations are non-specific, detection of mutations can be very time-consuming and expensive.

Myriad's BRACAnalysis (Myriad Genetic Laboratories, 2012) involves:

DNA sequencing of BRCA1—5400 base pairs which requires 35 PCR reactions in forward and reverse directions

DNA sequencing of BRCA2—10,200 base pairs which requires 47 PCR reactions in forward and reverse directions

Detection of 5 common genetic rearrangements using recombination specific

PCR, quantitative PCR or microarray comparative genomic hybridization (CGH).

a. 3.8 kb deletion of exon 13

b. 510 bp deletion of exon 22

c. 6 kb duplication of exon 13

d. 7.1 duplication of exons 8 and 9 and

e. 26 kb deletion of exons 14-20

CGH can detect rearrangements not included in the set of 5 using a microarray of 1700 probes.

The embodiments of the present invention, based on analysis technology, afford the ability to carry out large numbers of nucleic acid amplification processes in parallel and relatively quickly. With low-cost devices, the test can be repeated any number of times to check reproducibility.

ERBB2 overexpression: ERBB2 (formerly HER2/neu) is a non-hereditary oncogene found on chromosome 17 coding for the receptor tyrosine kinase 2 (erb-b2 protein). ERBB2 is particularly susceptible to mutation and overexpression in tumor cells and as such is a valuable marker for indication of prognosis and prediction of response to chemotherapy. Thus, gene copy number is a very good indicator of breast cancer status (Ross, et al., 2003).

Primers for Quantitative real-time PCR for ERBB2 are given in the following. (Koenigshoff, Wilhelm, Bohle, Pingoud, & Hahn, 2003)

| | | |
|---|---|---|
| neu-F | GAACTGGTGTATGCAGATTGC | 967-987 |
| neu-R | AGCAAGAGTCCCCATCCTA | 1051-1069 |
| neu-up | GTATGCACCTGGGCTCTTTGCAGGTCTCT-FAMc | 992-1020 |
| neu-down | CCGGAGCAAACCCCTATGTCCACAAGG-p | 1021-1047 |
| LCRed640- | | |

Receptor status by PCR: Apart from testing for mutations in the above genes, diagnostic testing for receptor status would afford valuable information for breast cancer patients. Receptor status, i.e. whether tumor cells have receptors or not, gives an indication of response to targeted chemotherapy. ER, PR and erb-b2 are the most important hormonal receptors and positive or negative status refers to the presence or absence of these receptors on tumor cells. A triple negative status is one in which none of the three receptors are present and this would be considered as a difficult tumor type to treat.

Currently, immunohistochemistry (IHC) staining with microscopic examination is the method that is widely used in pathology laboratories. However, a lack of consistency among laboratories due to variability in processing raises questions about the reliability of IHC.

While PCR-based tests are directly applicable to the assay devices, which are the subject of this invention, this type of testing has only recently been validated (proof of concept) as an alternative to IHC (Garuti, et al., 2014). Therefore, a first diagnostic, prognostic, or analytical objective for the assay devices, which are the subject of this invention, will be the testing for determination of the ER, PR and erb-b2 status, adapting methods from Garuti et al. or other suitable methods. The Garuti method is as follows:
RNA is extracted from the cell sample using commercial RNAeasy Micro Kit (Qiagen) or Trizol.
Total RNA is reverse transcribed using random hexamers (Takara, Shuzo Company Ltd, Shiga, Japan) in RT buffer containing RNAse inhibitor (Genenco M-Medical) into cDNA
The ER, PR and ERBB2 genes are amplified by real-time quantitative PCR using specific primers
QRT-PCR is used to measure the gene expression levels by means of

TaqMan Gene Expression Assays (Applied Biosystems Monza, Italy) and using RPLPO as the internal reference gene. Primers and probes were obtained from Applied Biosystems
QRT-PCR protocol:
   Activation (50° C. for 2 minute, 95° C. for 10 minute)
   Amplification and quantification 50 cycles (95° C. for 15 s, 60° C. for 1 minute with single fluorescence measurement)

The assay devices, which are the subject of this invention, employ the detection of emission by one or more electrochemiluminescence resonance energy transfer probes to detect the nucleic acid sequences complementary to the said probes. The design of the assay system used, including the probes, will be detailed below.

The assay system: The preferred sample type for the assay devices, which are the subject of this invention, is NAF, which contains epithelial cells detached from the walls of the breast ducts. NAF extraction is relatively painless for the patient but compared to the more painful process of ductal lavage the yield of epithelial cells is a few orders of magnitude lower at around 120 cells/breast compared to 13,500 cells per duct (Flanagan, Love, & Hwang, 2010). The invention keeps sample preparation to a minimum such that the NAF, once obtained, is preferably taken up directly by the assay device without any further preprocessing or prior storage. Once in the assay device it encounters lysis reagents that break the cell membranes releasing nucleic acids.

The preferred approach is chemical lysis rather than thermal lysis to minimize energy requirements. Detergents are used as lysis reagents to break down cell membranes. Ionic detergents such as SDS are fast acting, but they also destroy protein 3D structure, which will be detrimental to downstream enzyme-catalyzed amplification processes. This is important, as detergents will be carried over into the amplification chamber.

Milder non-ionic detergents such as Triton X-100 and NP-40 are proven to be effective and preserve protein structure. In particular, these are compatible with our preferred isothermal amplification systems.

Chaotropic salts including guanidinium hydrochloride or guanidinium isothiocyanate are also used for efficient nucleic acid extraction.

For the assay devices, which are the subject of this invention, a combination of either of these lysis reagents are compatible with the downstream amplification systems.

The following reagents are preferably used with the nucleic acid amplification steps in the assay devices:
Chaotropic salts such as guanidinium salts
Any non-ionic detergent
Nonidet P-40 lysis buffer (20 mM HEPES, pH 7.5/120 nM KCl/5 nM MgCl₂/1 mM dithiothreitol (DTT)/10% v/v glycerol/0.5% Nonidet P-40)
10 mM Tris-HCl, pH 7.4/0.5% SDS/10 mM EDTA, pH 7.4/10 mM NaCl without proteinase K supplementation
Serine and cysteine protease inhibitors—Roche cOmplete, Mini, EDTA-free Protease Inhibitor Cocktail tablets (contains AEBSF—4-(2-aminoethyl)benzenesulfonylfluoride). EDTA inhibits metalloproteases.

Nucleic acid amplification: For rapid result output in nucleic acid based systems, it is essential that nucleic acid amplification be as fast as possible. Isothermal amplification methods are advantageous over PCR because no thermal cycling is required; so energy and cooling demands are lower; simpler device architecture is required; and they are more tolerant of inhibitors such as heme, IgG, lactoferrin, heparin, urea and acidic polysaccharides.

Isothermal amplification methods like NEAR, EXPAR and RPA are preferable. Alternatives isothermal amplification methods are LAMP (Riken), HDA (BioHelix), Smart Amp (Riken), DNAble (Envirologix), and NASBA.

HDA, RPA, SMAP2, and NEAR act directly on double stranded DNA whereas other methods still require an initial heating at 95° C. to denature the double helix.

Temperatures are between 30° C. and 65° C. with higher temperatures favoring specificity. The analysis module is configured to detect the presence or absence of the at least an analyte in the sample, wherein the assay device is configured to perform an assay utilizing an “amplification master mix”, the amplification master mix including an enzyme initially preloaded in the assay device in dry form. The preloaded enzyme may be preloaded in the reagent chamber 830 (best shown in FIG. 42). The amplification reaction is carried out at a temperature range of between 30° C. to 65° C., with the amplification preferably carried out at a temperature range of between 55° C. to 59° C. Preference is for a mid-range temperature, e.g., Smart Amp (41° C., but slow), NEAR (53° C., 10 minutes), and RPA (30° C. to 42° C., 20 minutes). In one example, the temperature of amplification is about 56° C.

Hybridization: The molecular design of detector components within a detection array preferably conforms to the following general structures:

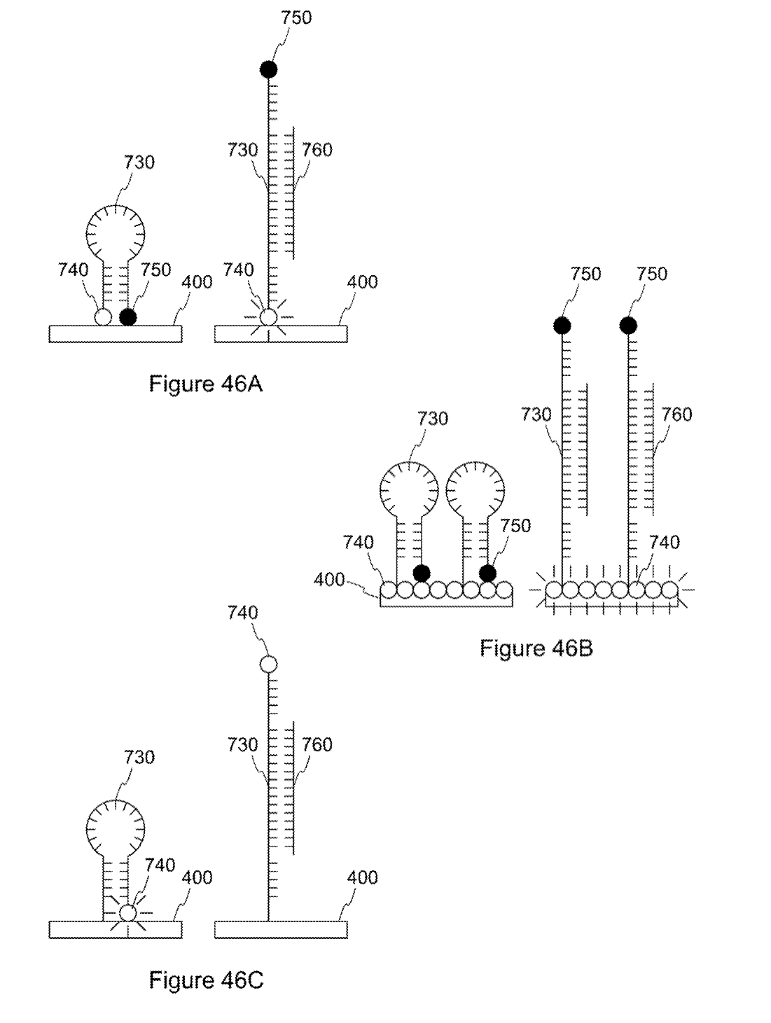

An immobilized nucleic acid hairpin probe 730 (depicted in FIG. 46A) has a luminophore 740 attached at one end and quencher 750 at the other end—molecular beacon. In this case, hybridization results in displacement of the quencher 750 from the luminophore 740, and then suitable electrical excitation leads to emission of photons. The luminophore 740 remains close to the electrode surface while the quencher 750 is displaced away from the electrode.

An immobilized nucleic acid hairpin probe 730 (depicted in FIG. 46B) has a quencher 750 attached at one end and luminophore 740 immobilized to the electrode surface (Yin, Dong, & Wang, 2004). Hybridization results in displacement of the quencher 750 from the luminophore 740, and then suitable electrical excitation leads to emission of photons.

An immobilized nucleic acid hairpin probe 730 (depicted in FIG. 46C) is attached at one end to the electrode and to luminophore 740 at the other end. With no hybridization, the suitable electrical excitation leads to emission of photons. Hybridization results in displacement of the luminophore 740 from the electrode surface and switching the emission off. In this case, there is no quencher 750, and reduction in electrochemiluminescence is a result of the distance the luminophore 740 is displaced from the electrode surface (Zhang, Qi, Li, Gao, & Zhang, 2008). The effectiveness of this approach is seen for loop lengths ranging from 16 bases to 21 bases (5.3 nm to 6.9 nm of duplex DNA, i.e. the length of the hybridized region). Beyond that, hybridization efficiency decreases.

Luminophores: While ruthenium bipyridyl complexes are used as electrochemiluminescence luminophores, a number of alternative classes of compound are more preferably used in certain embodiments of the invention that have advantages such as reduced cost, enhanced electrochemiluminescence efficiency, and enhanced stability. These compounds comprise any one of a plurality acridinium esters, water-soluble diphenylanthracene derivatives, and quantum dots.

Quenchers: Suitable quenchers 750 need to have high efficiency and be stable towards high applied voltages. The preferred quencher 750 is ferrocene, but Cy5 is also suitable. Black hole quenchers 750 have a tendency to undergo electrochemical bleaching making it less preferable.

Probe immobilization: There are two parts to probe adhesion: anchoring and encapsulation. This is achieved via a two-step process in which the probe is reacted with the electrode surface via groups with high affinity for the materials. This is followed by a drying step and then a second layer of a protective encapsulant is “overprinted” or dispensed on the bound probe. Again, this is followed by drying. In embodiments that require passivation of the regions between the probes, a further treatment via spotting is carried out followed by drying. Such a treatment is preferably an SDS or a BSA solution. The overall effect is to print a pattern on the surface rather than to lay down thin films.

A number of approaches are available for immobilizing nucleic acid probes to a surface, and this depends on what type of material is used. It is desirable to bind nucleic acid probes directly onto an electrode surface rather than on the chamber walls, because electrochemiluminescence is diffusion controlled and relies on interaction of reactive species that are generated at the electrode surface. In general, reactive species need to be within 10 nm (Debye length) of one another for reaction to give an emitting species. The principle behind the manner in which the probes work is based upon the distance the luminophore 740 is from both the quencher 750 and the electrode. If the coreactant is free in solution then the only issue is accessibility to the electrode and luminophore 740.

In the embodiments that require probe immobilization, complementary pairs of acceptor and donor groups that associate with a high binding constant, e.g. biotin with streptavidin or avidin, are used. More preferred embodiments use functional groups that have a high affinity for the native electrode surface or native “inter-electrode” surfaces and, therefore, bind rapidly without the need for added reagents and processing steps.

Layer-by-layer assembly on an electrode surface is employed in some embodiments to provide suitable functionality for probe immobilization. One of the preferred immobilization methods comprises adsorption via the negatively charged phosphate backbone onto cationic thin films on electrode surfaces. These may be built up by a combination of polymers and films selected from chitin, chitosan, PDA, poly-lysine, nafion, polyallylamine hydrochloride, sodium polystyrene sulfonate, polyvinylpyrrolidone, halloysite nanoclay and other charged clays such as attapulgite, palygorskite, bentonite, or smectite. Other surface modifiers include, but are not limited to, conducting polymers such as PANI, polypyrrole, PEDOT, PEDOT/PSS and self-assembled monolayers.

The use of anchoring groups such as catechols, phosphates, and phosphonates are favored as they bind strongly to hydroxylated surfaces such as silicon dioxide inter-electrode surfaces of some of the embodiments. Sulfur-based anchoring groups are used for binding to gold electrodes of many of the embodiments.

Another immobilization method comprises covalent attachment. Instantaneous reactions of boronic acids, boronate esters, phosphonates or catechols with hydroxylated surfaces are preferred methods. However, immobilization via coupling chemistry is not generally well suited to dispensing via spotting and, therefore, is not preferred. Silanization is also not preferred due to the high temperatures required for bond formation.

Reagent loading and storage: Introduction of the probes and any of the reagents within the analysis chip needs to be as simple as possible to allow for automation. The preferred approach is to deliver the probes and any of the reagents as premixed solutions via a robotic microdispenser and remove the solvent via evaporation or freeze-drying. In the case of sensitive biomolecules such as oligonucleotides, primers, probes and enzymes, a protective matrix is also preferably used for long-term storage, for as long as 12 months, at ambient temperature, and this forms part of the initial formulation. Upon drying, the matrix encapsulates the biomolecule which is readily reconstituted once exposed to an incoming biochemical or chemical mix, thereby releasing the biomolecule to perform its function. Any heating required as part of the mixing, lysing, amplification, and hybridization procedures will assist the rehydration phase.

Preferably, amplification enzymes, primers, NTPs, and probes are stored in the assay device in a dry form. Preferably, a hydroxyl-rich preservative such as Biomatrica or trehalose is incorporated in the formulations to give sufficient adhesion to the chamber surfaces.

The ability of trehalose to protect biomolecules can be attributed to its interaction with the minor groove of nucleic acid structures via hydrogen bonding. A reversible transition between its crystalline forms contributes to the replacement of water molecules around protein/membranes preserving the biomolecules' three-dimensional structure (Jain & Roy, 2009).

One of a number of commercially available products is used to store temperature labile biomolecules for long periods of time at room temperature.

Preferably, one of such products is used in the assay device to at least minimize but more advantageously eliminate the need for low-temperature storage.

One such product is Biomatrica's SampleMatrix®. The application utilizes a preservative matrix that protects nucleic acids from heat, UV and oxidation. Reagents are treated with a solution of the preservative and then are dried. Rehydration is simply carried out by the addition of water or buffer prior to analyses. Components of the kits are compatible with nucleic acid amplification processes (Wan, et al., 2010) thus avoiding the need for additive removal or further purification.

Preferred assay: NAF is collected from a patient either manually or by using a breast pump such as those supplied by Atossa Genetics or Halo Healthcare. The volume of NAF can be in the range of 1 μl to 500 μl but only as little as 1 μl is required for the assay performed in the assay devices, which are the subject of this invention. This is sampled via, e.g., the assay device 500 or the assay device 800 whereupon it undergoes the assay processes as detailed earlier in this specification. The NAF optionally undergoes dilution and the cellular content of the biochemical mix incorporating the NAF undergoes lysis.

By way of an example, Nonidet P-40 lysis buffer (20 mM HEPES, pH 7.5/120 nM KCl/5 nM MgCl₂/1 mM dithiothreitol (DTT)/10% v/v glycerol/0.5% Nonidet P-40) is the one or more of the liquid reagents 845, which are preloaded in one of the syringe pumps and is used to dilute the incoming NAF sample.

As another example, dried guanidinium isothiocyanate is the one or more assay reagents 840 preloaded in the syringe pump and dissolved by the incoming NAF sample by mild heating.

Also included among the preloaded one or more assay reagents are the isothermal nucleic acid amplification reagents. As these are compatible with the lysis reagent, both steps are carried out in a single biochemical mix.

In one embodiment, the amplification master mix is a DNAble amplification master mix including a nicking enzyme as supplied by Envirologix and is initially preloaded in dry form and the amplification reaction is carried out at a temperature range of between 55° C. to 59° C. for a period of 10 minutes or less, preferably at 56° C. for a period of 10 minutes or less. Other commercially available isothermal amplification mixes may also be used.

On completion of the amplification, the amplicon is released into the analysis module past the hydrogel valve isolating each detection chamber.

The analysis module has an array of detection chambers with preloaded molecular beacon probes. These probes are strongly adsorbed to gold electrodes via thiol anchoring groups on the 3′-end and a luminophore 740 such as a tris(bipyridyl)ruthenium complex on the 5′-end. Due to the hairpin structure, the luminophore 740 is bound in close proximity to the gold complex and is in an emitting state when there is a current flow between the electrodes. The molecular beacon probes contain specific sequences in the hairpin loop region that are complementary to the amplicon target sequences.

Upon successful hybridization of the amplicons with the thermally opened hairpin probe 730, failure to readopt the hairpin structure constitutes a positive test and loss of luminophore 740 emission. It is recognized that the configuration of the luminophore 740 on the molecular beacon can be of an alternative design such that a positive test results in a change from non-emission to emission. In this case, alternative anchoring chemistry is adopted such as biotin-streptavidin coupling.

The resulting change in emission is detected by an on-chip CMOS image sensor situated directly below the electrodes. This enables a high-resolution image of the array to be acquired in real time. This is important for quantitative analysis when multiple images need to be acquired over a period of time at fixed intervals.

The system and its components described above are purely illustrative and the skilled worker in this field will readily recognize many variations and modifications, which do not depart from the spirit and scope of the broad inventive concept.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

Although a preferred embodiment has been described in detail, it should be understood that various changes, substitutions, and alterations can be made by one of ordinary skill in the art without departing from the scope of the present invention.

It will be appreciated that various forms of the invention may be used individually or in combination.

The reference in this specification to any prior publication (or information derived from it), or to any matter which is known, is not and should not be taken as an acknowledgment or admission or any form of suggestion that the prior publication (or information derived from it) or known matter forms part of the common general knowledge in the field of endeavor to which this specification relates.

1. (canceled)

2. A device for analyzing a liquid sample, the device comprising:
a disposable part including:
an inlet for receiving the sample;
a reaction chamber;
an analysis module;
one or more flow paths arranged so as to provide a fluid flow path between the inlet and the reaction chamber, and a fluid flow path between the reaction chamber and the analysis module; and
at least one pump for moving fluid within the one or more flow paths; and
a reusable part that is releasably engageable with the disposable part, the reusable part including at least one actuator for operating the at least one pump of the disposable part.

3. The device of claim 2, wherein the at least one pump is at least one syringe pump, including a syringe barrel.

4. The device of claim 3, wherein one of the at least one syringe barrels forms the reaction chamber, wherein the device includes a heater to heat the contents of the reaction chamber, and/or wherein the device includes a syringe pump heating sleeve to heat the contents of the reaction chamber.

5.-6. (canceled)

7. The device of claim 2, wherein the device includes at least one sensor configured to detect the presence or absence of liquid within the one or more flow paths.

8.-9. (canceled)

10. The device of claim 2, wherein the analysis module is configured to detect the presence or absence of at least one analyte in the sample, and wherein the at least one analyte is a nucleic acid.

11. The device of claim 2, wherein the analysis module includes at least one photosensor.

12. The device of claim 10, wherein the analysis module is configured to detect the presence or absence of the at least one analyte using electrochemiluminescence.

13. The device of claim 2, wherein the analysis module includes one or more detection chambers.

14. The device of claim 13, wherein one or more of the detection chambers includes an electrochemiluminescence resonance energy transfer probe.

15. The device of claim 13, wherein the analysis module includes at least one heater to heat the contents of one or more of the detection chambers.

16. The device of claim 2, wherein the analysis module includes a plurality of detection chambers.

17. The device of claim 2, wherein the analysis module includes at least 47 detection chambers.

18. The device of claim 16, wherein each detection chamber includes a probe specific to a particular analyte.

19. The device of claim 13, wherein each detection chamber includes a valve closable to seal the detection chamber.

20. The device of claim 19, wherein the valve is a hydrogel valve.

21. (canceled)

22. The device of claim 2, wherein the sample includes Nipple Aspirate Fluid (NAF).

23. (canceled)

24. The device of claim 2, wherein the device includes at least 2 pumps, wherein one of the at least two pumps is operable to draw fluid via suction through the analysis module, and wherein the flow path between the inlet and the reaction chamber includes a dry reagent chamber.

25.-26. (canceled)

27. The device of claim 2, further comprising a communication interface that permits data transfer between the device and a processing system, wherein the device generates one or more signals indicative of the presence or absence of the at least one analyte, the one or more signals to be received by the processing system.

28. (canceled)

29. The device of claim 2, wherein the device is configured to amplify a nucleic acid or part thereof in the reaction chamber via an isothermal amplification reaction that is carried out for a period of 10 minutes or less, utilizing an amplification master mix, the amplification master mix including an enzyme initially preloaded in the device in dry form, wherein the amplification is carried out at a temperature range of between 30° C. to 65° C. and includes a nicking enzyme.

30.-36. (canceled)

37. The device of claim 2, wherein the analysis module is produced by a microfabrication process, and the analysis module includes:
a substrate; and
a patterned hydrogel layer that provides one or more hydrogel valve actuators, and optionally wherein the analysis module further comprises:
a cap foundation layer including a deep ultraviolet (DUV) photopatternable thermoplastic layer; and
a chip cap including a thermoplastic layer, wherein the substrate includes a silicon layer.

38.-39. (canceled)
